(19) United States
(12) Patent Application Publication
(10) Pub. No.: US 2023/0183381 A1
(43) Pub. Date: Jun. 15, 2023

(54) MULTISPECIFIC ANTIBODIES TARGETING CD38 AND BCMA AND USES THEREOF

(71) Applicant: Virtuoso BINco, Inc., San Mateo, CA (US)

(72) Inventors: Xiaocheng CHEN, San Mateo, CA (US); Leonard POST, San Mateo, CA (US)

(21) Appl. No.: 17/998,527

(22) PCT Filed: May 12, 2021
(86) PCT No.: PCT/IB2021/000375

§ 371 Date: Nov. 11, 2022

(30) Foreign Application Priority Data
May 12, 2020 (WO) PCT/IB2020/000374

(51) Int. Cl. C07K 16/46; A61P 35/00

(52) U.S. Cl. CPC C07K 16/468; A61P 35/00; C07K 2317/31; C07K 2317/35; C07K 2317/565; C07K 2317/52; C07K 2317/732; C07K 2317/734

(57) ABSTRACT

Disclosed herein are multispecific antibodies comprising a CD38 binding domain and an BCMA binding domain. Further provided herein are methods of treating cancer comprising administering to a subject having cancer a pharmaceutical composition comprising a multispecific antibody comprising a CD38 binding domain and an BCMA binding domain.

### CROSS-REFERENCE

This application claims the benefit of International Patent Application No. PCT/IB2020/000374 filed May 12, 2020, which is entirely incorporated herein by reference for all purposes.

### SEQUENCE LISTING

The instant application contains a Sequence Listing which has been submitted electronically in ASCII format and is hereby incorporated by reference in its entirety. Said ASCII copy, created on May 7, 2021, is named 55429-728_602_SL.txt and is 173,929 bytes in size.

### SUMMARY

The present disclosure provides multispecific antibodies (e.g. bispecific antibodies) comprising a CD38-binding domain and an BCMA binding domain.

In one aspect, provided herein is a multispecific antibody that comprises a CD38 binding domain and a BCMA binding domain.

In some embodiments, said multispecific antibody is bispecific, trispecific, or tetraspecific. In some embodiments, said multispecific antibody is bispecific. In some embodiments, said multispecific antibody is bivalent, trivalent, or tetravalent. In some embodiments, said multispecific antibody is bivalent.

In some embodiments, said CD38 binding domain comprises an antibody, or functional fragment or functional variant thereof, that specifically binds CD38. In some embodiments, said antibody, or functional fragment or functional variant thereof, comprises a variable domain of an IgG heavy chain and a variable domain of an IgG light chain. In some embodiments, said variable domain of an IgG heavy chain comprises a variable domain of an IgG1, IgG2, IgG3, or IgG4 heavy chain; and said variable domain of an IgG light chain comprises a variable domain of an IgGl, IgG2, IgG3, or IgG4 light chain. In some embodiments, said variable domain of an IgG heavy chain comprises a variable domain of an IgG1 heavy chain; and said variable domain of an IgG light chain comprises a variable domain of an IgG1 light chain. In some embodiments, said antibody, or functional fragment or functional variant thereof, comprises a scFv or a Fab. In some embodiments, said variable domain of an IgG heavy chain (HC) of said CD38 binding domain comprises complementarity determining regions (CDRs): HC-CDR1, HC-CDR2, and HC-CDR3, wherein the HC-CDR1, the HC-CDR2, and the HC-CDR3 of said heavy chain variable domain comprise a set of sequences selected from the group consisting of the following set of sequences: HC-CDR1: SEQ ID NO: 9; HC-CDR2: SEQ ID NO: 10; HC-CDR3: SEQ ID NO: 11; and HC-CDR1: SEQ ID NO: 12; HC-CDR2: SEQ ID NO: 13; HC-CDR3: SEQ ID NO: 14. In some embodiments, said variable domain of an IgG light chain (LC) of said CD38 binding domain comprises complementarity determining regions (LC) (CDRs): LC-CDR1, LC-CDR2, and LC-CDR3, wherein the LC-CDR1, the LC-CDR2, and the LC-CDR3 of said heavy chain variable domain comprise a set of sequences selected from the group consisting of the following set of sequences: LC-CDR1: SEQ ID NO: 67; LC-CDR2: SEQ ID NO: 68; LC-CDR3: SEQ ID NO: 69; and LC-CDR1: SEQ ID NO: 70; LC-CDR2: SEQ ID NO: 71; LC-CDR3: SEQ ID NO: 72. In some embodiments, said variable domain of an IgG light chain (LC) of said CD38 binding domain comprises complementarity determining regions (CDRs): LC-CDR1, LC-CDR2, and LC-CDR3, and said variable domain of an IgG heavy chain (HC) of said CD38 binding domain comprises CDRs: HC-CDR1, HC-CDR2, and HC-CDR3, wherein said LC: LC-CDR1, LC-CDR2, and LC-CDR3 sequences and said HC: HC-CDR1, HC-CDR2, and HC-CDR3 sequences are as set forth in any one of the following sets of sequences: HC-CDR1: SEQ ID NO: 9; HC-CDR2: SEQ ID NO: 10; HC-CDR3: SEQ ID NO: 11; LC-CDR1: SEQ ID NO: 67; LC-CDR2: SEQ ID NO: 68; and LC-CDR3: SEQ ID NO: 69; or HC-CDR1: SEQ ID NO: 12; HC-CDR2: SEQ ID NO: 13; HC-CDR3: SEQ ID NO: 14; and LC-CDR1: SEQ ID NO: 70; LC-CDR2: SEQ ID NO: 71; and LC-CDR3: SEQ ID NO: 72. In some embodiments, said variable domain of an IgG heavy chain of said CD38 binding domain comprises an amino acid sequence with at least 90%, 95%, 96%, 97%, 98%, 99%, or 100% identity to any one of SEQ ID NO: 114-136, 197-209. In some embodiments, said variable domain of an IgG light chain of said CD38 binding domain comprises an amino acid sequence with at least 90%, 95%, 96%, 97%, 98%, 99%, or 100% identity to any one of SEQ ID NO: 137-162, 210-218. In some embodiments, said CD38 binding domain comprises a variable domain of an IgG heavy chain comprises an amino acid sequence with at least 90%, 95%, 96%, 97%, 98%, 99%, or 100% identity to SEQ ID NO: 114; and a variable domain of an IgG heavy chain comprises an amino acid sequence with at least 90%, 95%, 96%, 97%, 98%, 99%, or 100% identity to SEQ ID NO: 137; or a variable domain of an IgG heavy chain comprises an amino acid sequence with at least 90%, 95%, 96%, 97%, 98%, 99%, or 100% identity to SEQ ID NO: 115; and a variable domain of an IgG heavy chain comprises an amino acid sequence with at least 90%, 95%, 96%, 97%, 98%, 99%, or 100% identity to SEQ ID NO: 138.

In some embodiments, said BCMA binding domain comprises an antibody, or functional fragment or functional variant thereof, that specifically binds BCMA. In some embodiments, said antibody, or functional fragment or functional variant thereof, comprises a variable domain of an IgG heavy chain and a variable domain of an IgG light chain. In some embodiments, said variable domain of an IgG heavy chain comprises a variable domain of an IgG1, IgG2, IgG3, or IgG4 heavy chain; and said variable domain of an IgG light chain comprises a variable domain of an IgG1, IgG2, IgG3, or IgG4 light chain. In some embodiments, said variable domain of an IgG heavy chain comprises a variable domain of an IgG1 heavy chain; and said variable domain of an IgG light chain comprises a variable domain of an IgG1 light chain. In some embodiments, said antibody, or functional fragment or functional variant thereof, comprises a scFv or a Fab. In some embodiments, said variable domain of an IgG heavy chain (HC) of said BCMA binding domain comprises complementarity determining regions (CDRs): HC-CDR1, HC-CDR2, and HC-CDR3, wherein said HC-CDR1, the HC-CDR2, and the HC-CDR3 of said variable heavy chain comprise a set of sequences selected from the group consisting of the following set of sequences: HC-CDR1: SEQ ID NO: 1; HC-CDR2: SEQ ID NO: 2; HC-CDR3: SEQ ID NO: 3. In some embodiments, said variable domain of an IgG light chain (LC) of said BCMA binding domain comprises complementarity determining regions (CDRs): LC-CDR1, LC-CDR2, and LC-CDR3, wherein the LC-CDR1, the LC-CDR2, and the LC-CDR3 of the heavy chain comprise a set of sequences selected from the group consisting of the following set of sequences: LC-CDR1: SEQ ID NO: 4; LC-CDR2: SEQ ID NO: 5; LC-CDR3: SEQ ID NO: 6. In some embodiments, said variable domain of an IgG light chain (LC) of said BCMA binding domain comprises complementarity determining regions (CDRs): LC-CDR1, LC-CDR2, and LC-CDR3, and said variable domain of an IgG heavy chain (HC) of said BCMA binding domain comprises complementarity determining regions HC-CDR1, HC-CDR2, and HC-CDR3, wherein said LC: LC-CDR1, LC-CDR2, and LC-CD3 sequences and said HC: HC-CDR1, HC-CDR2, and HC-CDR3 sequences comprise sequences with at least 90%, 95%, 96%, 97%, 98%, 99%, or 100% identity to sequences as set forth in any one of the following sets of sequences: HC-CDR1: SEQ ID NO: 1; HC-CDR2: SEQ ID NO: 2; HC-CDR3: SEQ ID NO: 3; LC-CDR1: SEQ ID NO: 4; LC-CDR2: SEQ ID NO: 5; and LC-CDR3: SEQ ID NO: 6. In some embodiments, said variable domain of an IgG heavy chain of said BCMA binding domain comprises an amino acid sequence with at least 90%, 95%, 96%, 97%, 98%, 99%, or 100% identity to SEQ ID NO: 7. In some embodiments, said variable domain of an IgG light chain of said BCMA binding domain comprises an amino acid sequence with at least 90%, 95%, 96%, 97%, 98%, 99%, or 100% identity to SEQ ID NO: 8. In some embodiments, wherein said BCMA binding domain comprises a variable domain of an IgG heavy chain comprises an amino acid sequence with at least 90%, 95%, 96%, 97%, 98%, 99%, or 100% identity to SEQ ID NO: 7; and a variable domain of an IgG heavy chain comprises an amino acid sequence with at least 90%, 95%, 96%, 97%, 98%, 99%, or 100% identity to SEQ ID NO: 8.

In some embodiments, said multispecific antibody further comprises an IgG hinge region, or a portion thereof. In some embodiments, said multispecific antibody further comprises an Fc region. In some embodiments, said Fc region comprises an IgG CH2 domain and an IgG CH3 domain. In some embodiments, said IgG hinge region is C terminal to said CD38 binding domain or said BCMA binding domain and N terminal to said Fc region. In some embodiments, said Fc region comprises a heterodimeric Fe region.

In some embodiments, said Fc region comprises at least one amino acid modification that increases the half-life of the multispecific antibody. In some embodiments, said Fc region comprises at least one amino acid modification that modulates its interaction with an Fc receptor. In some embodiments, said Fc region comprises at least one amino acid modification that increases binding of said Fc region to an Fc receptor.

In some embodiments, said Fc region comprises at least one amino acid modification that decreases glycosylation of the Fc region. In some embodiments, said modification is an amino acid substitution, deletion, or addition. In some embodiments, said modification is an amino acid substitution. In some embodiments, said at least one amino acid modification that decreases glycosylation of the Fc region comprises an amino acid substitution at a position corresponding to position N297 of human IgG1, wherein the numbering is according to the EU index of Kabat.

In some embodiments, said Fc region is afucosylated.

In some embodiments, said Fc region comprises at least one amino acid modification that increases antibody-dependent cellular cytotoxicity (ADCC).

In some embodiments, said modification is an amino acid substitution, deletion, or addition. In some embodiments, said modification is an amino acid substitution. In some embodiments, said Fc region comprises at least one mutation that increases antibody-dependent cellular cytotoxicity (ADCC), wherein said at least one mutation that increases ADCC comprises an amino acid substitution at positions corresponding to positions S239, 1332, and A330 of human IgG1, wherein the amino acid numbering is according to the EU index according to Kabat et al. In some embodiments, said amino acid substitutions are S239D, I332E, and A330L, wherein the amino acid numbering is according to the EU index according to Kabat et al.

In some embodiments, said multispecific antibody comprises a heterodimeric Fc region, wherein said heterodimeric Fc region comprises a knob chain and a hole chain, forming a knob-in-hole (KM) structure. In some embodiments, said knob chain comprises an amino acid substitution at a position corresponding to T366 of IgG1, wherein amino acid position numbering is according to the EU index according to Kabat et al. In some embodiments, said T366 substitution comprises a T336W mutation, wherein amino acid position numbering is according to the EU index according to Kabat et al. In some embodiments, said hole chain comprises an amino acid substitution at a position corresponding to T366, L368, or Y407 of IgG1, wherein amino acid position numbering is according to the EU index according to Kabat et al. In some embodiments, said hole chain comprises an amino acid substitution at a position corresponding to T366, L368, and Y407 of IgG1, wherein amino acid position numbering is according to the EU index according to Kabat et al. In some embodiments, said T366, L368, or Y407 amino acid substitutions comprise a T366S, L368A, or Y407V of IgG1, wherein amino acid position numbering is according to the EU index according to Kabat et al. In some embodiments, said T366, L368, and Y407 amino acid substitutions comprises a T366S, L368A, and Y407V of IgG1, wherein amino acid position numbering is according to the EU index according to Kabat et al.

In some embodiments, said multispecific antibody has a higher affinity for CD38 expressed on the surface of a cancer cell relative to a protein that comprises said CD38 binding domain but lacks an BCMA binding domain. In some embodiments, said multispecific antibody binds to a target cell that expresses CD38 and BCMA with an enhanced affinity compared to that of a protein that comprises only the first component that specifically binds to CD38 or the second component that specifically binds to BCMA. In some embodiments, said multispecific antibody exhibits a higher affinity for CD38 expressed on the surface of a target cancer cell than for CD38 expressed on the surface of a non-cancer cell. In some embodiments, BCMA is expressed on the surface of said target cancer cell, wherein said binding is measured by flow cytometry. In some embodiments, said target cell expresses CD38 and BCMA. In some embodiments, said target cell expresses a lower level of CD38 relative to BCMA on the surface of said target cell. In some embodiments, the ratio of CD38 to BCMA on the surface of said cancer cells is at least about 1, 1.5, 2.0, 2.5, 5, 10, 15, 20, 50, 100, or 200. In some embodiments, said target cell is a cancer cell. In some embodiments, said cancer is a hematological malignancy. In some embodiments, said hematological malignancy is a B cell malignancy. In some embodiments, said B cell malignancy is multiple myeloma. In some embodiments, said target cell is a plasma cell. In some embodiments, said plasma cell secretes antibodies that recognize autoantigens.

In some embodiments, said multispecific antibody exhibits a higher affinity for CD38 expressed on the surface of a target plasma cell than for CD38 expressed on the surface of a non-plasma cell. In some embodiments, said multispecific antibody exhibits a higher affinity for CD38 expressed on the surface of a target plasma cell than for CD38 expressed on the surface of a non-plasma cell B cell. In some embodiments, said multispecific antibody exhibits a higher affinity for CD38 expressed on the surface of a target plasma cell that secretes antibodies that recognize an autoantigen than for CD38 expressed on the surface of a plasma cell that does not secrete antibodies that recognize an autoantigen. In some embodiments, said plasma cell is a plasmablast, differentiated plasma cell, or long lived plasma cell. In some embodiments, said plasma cell is a cancer cell. In some embodiments, said plasma cell secrete antibodies that recognize autoantigens.

In some embodiments, said multispecific antibody induces enhanced antibody-dependent cellular cytotoxicity (ADCC) activity on a target cell that expresses CD38 to BCMA compared to ADCC activity induced on said target cell by a monospecific antibody that comprises only one of the component that specifically binds to CD38 or the second component that specifically binds to BCMA. In some embodiments, said multispecific antibody induces at least 5%, 10%, 15%, 20%, 25%, 30%, 25%, 40%, 45%, or 50% more ADCC activity than said control monospecific antibody.

In some embodiments, said multispecific antibody induces enhanced complement-dependent cytotoxicity (CDC) activity on a target cell that expresses CD38 to BCMA compared to CDC activity induced on said target cell by a protein that comprises only one of the component that specifically binds to CD38 or the second component that specifically binds to BCMA. In some embodiments, said multispecific antibody induces at least 5%, 10%, 15%, 20%, 25%, 30%, 25%, 40%, 45%, or 50% more CDC activity than said control monospecific antibody.

In some embodiments, said multispecific antibody binds to a cancer cell that expresses CD38 and BCMA on the surface, and wherein the ratio of BCMA to CD38 on the surface of said cancer cell is at least about 1, 1.5, 2.0, 2.5, 5, 10, 15, 20, 50, 100, or 200.

In some embodiments, the multispecific antibody of any preceding claim, wherein said multispecific antibody binds to a plasma cell that expresses CD38 and BCMA on the surface, and wherein the ratio of BCMA to CD38 on the surface of said cancer cell is at least about 1, 1.5, 2.0, 2.5, 5, 10, 15, 20, 50, 100, or 200. In some embodiments, said plasma cell is a plasmablast, differentiated plasma cell, or long lived plasma cell. In some embodiments, said plasma cell is a cancer cell. In some embodiments, said plasma cell secrete antibodies that recognize autoantigens.

In one aspect, provided herein is a nucleic acid molecule encoding a multispecific antibody described herein.

In one aspect, provided herein is a vector comprising the nucleic acid molecule described herein.

In one aspect, provided herein is a pharmaceutical composition comprising a multispecific antibody described herein. In some embodiments, said pharmaceutical composition further comprises a pharmaceutically acceptable carrier, an excipient, or any combinations thereof.

In one aspect, provided herein are methods of treating a subject having cancer, said method comprising: administering to the subject a multispecific antibody described herein or a pharmaceutical composition described herein.

In some embodiments, said cancer comprises cancer cells that express CD38 and BCMA. In some embodiments, the ratio of BCMA to CD38 on the surface of said cancer cells is at least about 1, 1.5, 2.0, 2.5, 5, 10, 15, 20, 50, 100, or 200. In some embodiments, said cancer cells that express CD38 and BCMA are lysed. In some embodiments, said multispecific antibody induces antibody-dependent cellular cytotoxicity (ADCC) mediated killing of said cancer cells that express CD38 and BCMA. In some embodiments, said multispecific antibody induces complement-dependent cytotoxicity (CDC) mediated killing of said cancer cells that express CD38 and BCMA.

In some embodiments, the cancer is a hematological malignancy. In some embodiments, said cancer is B cell cancer. In some embodiments, said cancer is multiple myeloma.

In some embodiments, said method further comprises administering to said subject an anti-cancer agent. In some embodiments, said anti-cancer agent is a chemotherapeutic agent or a biologic agent.

In one aspect, provided herein is a method of treating a subject having an autoimmune disease, said method comprising: administering to the subject a multispecific antibody described herein or a pharmaceutical composition described herein.

In some embodiments, said autoimmune disease is characterized by immune cells that express CD38 and BCMA. In some embodiments, said immune cells are B cells. In some embodiments, said B cells are plasma cells. In some embodiments, said plasma cells comprise plasmablasts, differentiated plasma cells, or long lived plasma cells. In some embodiments, said immune cells secrete antibodies that recognize autoantigens.

In some embodiments, the ratio of BCMA to CD38 on the surface of said immune cells is at least about 1, 1.5, 2.0, 2.5, 5, 10, 15, 20, 50, 100, or 200. In some embodiments, said immune cells that express CD38 and BCMA are lysed. In some embodiments, said multispecific antibody induces antibody-dependent cellular cytotoxicity (ADCC) mediated killing of said immune cells that express CD38 and BCMA. In some embodiments, said multispecific antibody induces complement-dependent cytotoxicity (CDC) mediated killing of said immune cells that express CD38 and BCMA.

In some embodiments, said autoimmune disease is selected from the group consisting of rheumatoid arthritis (RA), systemic lupus erythematosus (SLE), multiple sclerosis (MS), pemphigus vulgaris (PV), myasthenia gravis (MG) and immune thrombocytopenic purpura (ITP).

### BRIEF DESCRIPTION OF THE FIGURES

FIG. 1 is a graphic depiction of a bispecific antibody described herein, which binds CD38 and BCMA. In the depicted embodiment, one antigen binding domain (e.g., CD38) can be a Fab and the other antigen binding domain can be a scFv (e.g., BCMA), and vice versa, i.e. CD38 antigen binding domain is a scFv and the BCMA binding domain is a Fab. In the depicted embodiment, the CH3 regions of the constant heavy chain polypeptides are engineered to form a knob-in-hole structure to mediate formation of the bispecific antibody.

FIG. 2A is a line graph showing the results of an ELISA testing the binding of anti-CD38 antibodies and anti-CD38×BCMA bispecific antibodies to human CD38.

FIG. 2B is a line graph showing the results of an ELISA testing the binding of anti-BCMA antibodies and anti-CD38×BCMA bispecific antibodies to human BCMA.

FIG. 3 is a line graph showing the results of an ADCC assay on Daudi B lymphoblasts (human Burkitt's lymphoma cell line). The graph plots percent cytotoxicity (y axis) versus the concentration of the indicated antibody (x axis). The data shows that two bispecific antibodies that bind to BCMA and CD38 exhibits higher ADCC mediated lysis of Daudi cells compared to a bivalent monospecific anti-BCMA antibodies, a bivalent monospecific anti-CD38 antibodies, and an isotype control human IgG1 antibody.

FIG. 4 is a line graph showing the results of an ADCC assay on huNS1 lymphoblasts (human myeloma cell line). The graph plots percent cytotoxicity (y axis) versus the concentration of the indicated antibody (x axis). The data shows that two bispecific antibodies that bind to BCMA and CD38 exhibits improved ADCC mediated lysis of huNS1 cells compared to a bivalent monospecific anti-BCMA antibodies, a bivalent monospecific anti-CD38 antibodies, and an isotype control human IgG1 antibody.

FIG. 5A shows a variable light chain amino acid sequence of antibody variants of the humanized h18E4-19 antibody that were generated by mutating each indicated amino acid residue to an alanine. FIG. 5A discloses SEQ ID NOS 137 and 210-218, respectively, in order of appearance.

FIG. 5B shows a variable heavy chain amino acid sequence of antibody variants of the humanized h18E4-19 antibody that were generated by mutating each indicated amino acid residue to an alanine. FIG. 5B discloses SEQ ID NOS 114 and 197-209, respectively, in order of appearance.

FIG. 6 is a line graph illustrating the results of an ELISA testing the binding of anti-CD38×BCMA bispecific antibody variants to human CD38.

FIG. 7A is a line graph illustrating the results of an ADCC assay on Daudi (human Burkitt's lymphoma cell line). The graph plots percent cytotoxicity (y axis) versus the concentration of the indicated antibody (x axis). The data shows that CD38×BCMA affinity variants exhibit higher ADCC mediated lysis of Daudi cells compared to a monovalent anti-BCMA antibodies, a bivalent monospecific anti-CD38 antibodies, and an isotype control human IgG1 antibody.

FIG. 7B is a line graph illustrating the results of an ADCC assay on U266B1 (human myeloma cell line). The graph plots percent cytotoxicity (y axis) versus the concentration of the indicated antibody (x axis). The data shows that CD38×BCMA affinity variants exhibit higher ADCC mediated lysis of U266B1 cells compared to a monovalent anti-BCMA antibodies, a bivalent monospecific anti-CD38 antibodies, and an isotype control human IgG1 antibody.

FIG. 8 illustrates mean volume(s) of NCI-H929 cell-derived xenograft tumors treated with CD38.BMK1 or anti-CD38 or anti-BCMA or CD38×BCMA bispecific antibodies.

### DETAILED DESCRIPTION

Populations of long lived plasma cells have been shown to secrete autoantibodies. The cell surface protein CD38 is expressed on bone marrow plasma cells, which are enriched in chronically inflamed tissues and secrete autoantibodies. While some anti-CD38 therapies, e.g., the anti-CD38 antibody, daratumumab, have been shown to deplete plasma cells/plasmablasts in PBMC from patients with autoimmune disorders; these long lived autoreactive plasma cells are largely resistant to current B-cell targeted therapies. Therefore, there is a need for new treatments that can target CD38 expressing autoreactive plasma cells, while overcoming the limitations of current anti-CD38 immunotherapies.

Populations of long lived plasma cells have been shown to secrete autoantibodies. He M, et al., Rationale of anti-CD19 immunotherapy: an option to target autoreactive plasma cells in autoimmunity, Arthritis Res Ther 2012; 14:S1; Hiepe F, et al., Long-lived autoreactive plasma cells drive persistent autoimmune inflammation, Nat Rev Rheumatol 2011; 7:170-178. The cell surface protein CD38 is expressed on bone marrow plasma cells, which are enriched in chronically inflamed tissues and secrete autoantibodies. He M, et al., A unique population of IgG expressing plasma cells lacking CD19 is enriched in human bone marrow, Blood 2015; 125:1739-1748. While some anti-CD38 therapies, e.g., the anti-CD38 antibody daratumumab, have been shown to deplete plasma cells/plasmablasts in PBMC from patients with autoimmune disorders; these long lived autoreactive plasma cells are largely resistant to current B-cell targeted therapies. Hiepe F, et al, Nat Rev Rheumatol 2011; 7:170-178; Cole S, et al., Integrative analysis reveals CD38 as a therapeutic target for plasma cell-rich pre-disease and established rheumatoid arthritis and systemic lupus erythematosus, Arthritis Research & Therapy 2018 20:85. Therefore, there is a need for new treatments that can target CD38 expressing autoreactive plasma cells, while overcoming the limitations of current anti-CD38 immunotherapies.

BCMA is expressed exclusively in B-cell lineage cells, particularly in the interfollicular region of the germinal center as well as on plasmablasts and differentiated plasma cells. BCMA does not appear to be critical for overall B-cell homeostasis but is required for optimal survival of long-lived plasma cells in the bone marrow. O'Connor B P, et al., BCMA is essential for the survival of long-lived bone marrow plasma cells, J. Exp. Med. 199(1), 91-98 (2004); Xu S, et al., B-cell maturation protein, which binds the tumor necrosis factor family members BAFF and APRIL, is dispensable for humoral immune responses, Mol. Cell. Biol. 21(12), 4067-4074 (2001).

The present disclosure is based, at least in part, on the discovery that multispecific antibodies comprising a CD38 binding domain and an BCMA binding domain can overcome the problems of monospecific bivalent anti-CD38 antibodies by providing an increased local concentration of the anti-CD38 antibody to relevant cell populations of autoreactive plasma cells; thereby increasing the potency and ADCC mediated killing of target autoreactive cells. The CD38×BCMA targeting multispecific antibodies described herein are also useful for improved methods of treating cancers expressing CD38 and BCMA, e.g., hematological malignancies, e.g., B cell lymphomas (e.g., Birkett's lymphoma); and myelomas (e.g., multiple myeloma).

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “antibody” and “immunoglobulin” are used interchangeably herein and are used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The terms “monoclonal antibody” and “mAb” are used interchangeably herein and refer to an antibody obtained from a substantially homogeneous population of antibodies, i.e., the individual antibodies of the population are identical except for possible naturally occurring mutations that may be present in minor amounts.

The terms “native antibodies” and “native immunoglobulins” are heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “hypervariable region,” as used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest Fifth Edition, U.S. Department of Health and Human Services, NIH Publication No. 91-3242 (referred to herein as “Kabat et al”) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Chothia and Lesk, (1987) J. Mol. Biol., 196:901-917). “Framework” or “FR” residues are those variable domain residues other than the hypervariable region residues, as herein deemed.

In some instances, the CDRs of an antibody is determined according to (i) the Kabat numbering system Kabat et al. (1991) Sequences of Proteins of Immunological Interest Fifth Edition, U.S. Department of Health and Human Services, NIH Publication No. 91-3242; or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273 :927-948; Chothia et al., 1992, J. Mol. Biol., 227:799-817; Tramontano A et al. , 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDRI), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “antibody fragment” as used herein refers to a molecule that comprises a portion of an intact antibody, preferably the antigen-binding or variable region of the intact antibody. Examples of antibody fragments include Fab, Fab, F(ab′)2, Fv fragments, and single chain fragment variable (scFv); diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

The term “Fv” as used herein refer to the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The term “Fab” refers to a protein that contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

As used herein, the term “antigen-binding site” refers to the part of the antigen binding molecule that specifically binds to an antigenic determinant. More particularly, the term “antigen-binding site” refers the part of an antibody that comprises the area which specifically binds to and is complementary to part or all of an antigen. Where an antigen is large, an antigen binding molecule may only bind to a particular part of the antigen, which part is termed an epitope. An antigen-binding site may be provided by, for example, one or more variable domains (also called variable regions). Preferably, an antigen-binding site comprises an antibody light chain variable region (VL) and an antibody heavy chain variable region (VH).

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17,323-329 (2000)), and traditional binding assays (Heeley, Endocr Res 28,217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10-7 M or less, e.g. from 10-7M to 10-13 M, e.g. from 10-9 M to 10-13 M).

Depending on the amino acid sequence of the constant domain of their heavy chains, immunoglobulins can be assigned to different classes. There are five major classes of human immunoglobulins: IgA, IgD, IgE, IgG, IgM, and IgY , and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity. The light chains of antibodies (immunoglobulins) from any vertebrate species can be assigned to one of two clearly distinct types, called kappa (κ) and lambda (λ), based on the amino acid sequences of their constant domains.

The term “chimeric antibody,” as used herein refers to an antibody in which a portion of the heavy and/or light chain is derived from a particular source (e.g., protein) or species, while the remainder of the heavy and/or light chain is derived from a different source (e.g., protein) or species.

The term “recombinant human antibody,” as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The term “valent” as used herein denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding sites and are bispecific. That is, the antibodies may be bispecific even in cases where there are more than two binding sites (i.e. that the antibody is trivalent or multivalent). In particular, the invention relates to bispecific bivalent antibodies, having one binding site for each antigen they specifically bind to.

The term “monospecific” antibody as used herein denotes an antibody that has one or more binding sites each of which bind to the same epitope of the same antigen. The term “bispecific” means that the antibody is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific antibody is an 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigenic determinant.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The terms “cancer” and “tumor” are used interchangeably herein, encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

As used herein, the terms “Antibody-dependent cellular cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell.

### BCMA Binding Domains

In some embodiments, disclosed herein is a multispecific antibody that comprises an anti-BCMA binding domain. In some embodiments, the BCMA binding domain comprises an antibody or antigen binding fragment or variant thereof. In some embodiments, antibody or antigen binding fragment or variant thereof is a monoclonal antibody. In some embodiments, antibody or antigen binding fragment or variant thereof is a human antibody, a murine antibody, a humanized antibody, or a chimeric antibody. In some embodiments, the BCMA binding domain comprises a monovalent Fab, a bivalent Fab′2, a single-chain variable fragment (scFv), or functional fragment or variant thereof. In some embodiments, the BCMA binding domain comprises an immunoglobulin variable heavy chain domain (VH). In some embodiments, the BCMA binding domain comprises an immunoglobulin variable light chain domain (VL). In some embodiments, the BCMA binding domain comprises a VH and a VL.

In some embodiments, the multispecific antibodies, and binding fragments thereof, are derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

### Complementarity Determining Regions (CDRs)

In some embodiments, the BCMA binding domain comprises an immunoglobulin variable heavy chain domain (VH) that comprises at least one, two, or three complementarity determining regions (CDRs) disclosed in Table 1 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises an immunoglobulin variable light chain domain (VL) that comprises at least one, two, or three complementarity determining regions (CDRs) disclosed in Table 2 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VH that comprises at least one, two, or three complementarity determining regions (CDRs) disclosed in Table 1 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises at least one, two, or three complementarity determining regions (CDRs) disclosed in Table 2 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VH that comprises a CDR1 of SEQ ID NO: 1, a CDR2 of SEQ ID NO: 2, and a CDR3 of SEQ ID NO: 3.

In some embodiments, the BCMA binding domain comprises a VL that comprises a CDR1 of SEQ ID NO: 4, a CDR2 of SEQ ID NO: 5, and a CDR3 of SEQ ID NO: 6.

In some embodiments, the BCMA binding domain comprises a VH that comprises a CDR1 of SEQ ID NO: 1, a CDR2 of SEQ ID NO: 2, and a CDR3 of SEQ ID NO: 3; and a VL that comprises a CDR1 of SEQ ID NO: 4, a CDR2 of SEQ ID NO: 5, and a CDR3 of SEQ ID NO: 6.

TABLE 1
VH CDR amino acid sequences of anti-BCMA antibodies as defined by
Kabat et al.

| | SEQ | | SEQ | | SEQ | |
|---|---|---|---|---|---|---|
| Antibody | ID NO | CDR1 | ID NO | CDR2 | ID NO | CDR3 |
| Anti- | 1 | NYWMH | 2 | ATYRGHSDTYYNQKFKG | 3 | GAIYNGYDVLDN |
| BCMA.BMK2 | | | | | | |

TABLE 2
VL CDR amino acid sequences of anti-BCMA antibodies as defined by
Kabat et al.

| | SEQ ID | | SEQ ID | | SEQ ID | |
|---|---|---|---|---|---|---|
| Antibody | NO | CDR1 | NO | CDR2 | NO | CDR3 |
| Anti- | 4 | SASQDISNYLN | 5 | YTSNLHS | 6 | QQYRKLPWT |
| BCMA.BMK2 | | | | | | |

In some embodiments, a CDR described herein comprises one, two, or three amino acid modifications. In some embodiments, said modification is a substitution, addition, or deletion. In some embodiments, a CDR described herein comprises one, two, or three conservative amino acid substitutions. In some embodiments, the one, two, or three amino acid modifications does not substantially modify binding to human BCMA. In some embodiments, the one, two, or three amino acid modifications modifies binding to human BCMA. In some embodiments, a VH-CDR3 and/or VL-CDR3 comprises an amino acid substitution that modifies binding to human BCMA, immunogenicity, or some other feature. In some embodiments, the amino acid substitution is an alanine (A).

### Variable Heavy and Variable Light Regions

In some embodiments, the BCMA binding domain comprises a VH that comprises an amino acid sequence disclosed in Table 3 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VL that comprises an amino acid sequence disclosed in Table 4 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VH that comprises an amino acid sequence disclosed in Table 3 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence disclosed in Table 4 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VH that comprises an amino acid sequence of SEQ ID NO: 7, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VL that comprises an amino acid sequence of SEQ ID NO: 8, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the BCMA binding domain comprises a VH that comprises an amino acid sequence of SEQ ID NO: 7, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 8, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

TABLE 3
Amino acid sequence of the anti-BCMA variable heavy chain binding
domains. CDRs according to Kabat et al. are underlined.

| | SEQ ID | |
|---|---|---|
| Name | NO | Amino Acid Sequence |
| Anti- | 7 | EVQLQQSGAVLARPGASVKMSCKGSGYTFTNYWMHWV |
| BCMA.BMK2 | | KQRPGQGLEWIGATYRGHSDTYYNQKFKGKAKLTAVTS |
| | | TSTAYMELSSLTNEDSAVYYCTRGAIYNGYDVLDNWGQ |
| | | GTLVTVSS |

TABLE 4
Amino acid sequence of the anti-BCMA variable light chain binding
domains. CDRs according to Kabat et al. are underlined.

| | SEQ ID | |
|---|---|---|
| Name | NO | Amino Acid Sequence |
| Anti- | 8 | DIQLTQTTSSLSASLGDRVTISCSASQDISNYLNWYQQKPD |
| BCMA.BMK2 | | GTVELVIYYTSNLHSGVPSRFSGSGSGTDYSLTIGYLEPED |
| | | VATYYCQQYRKLPWTFGGGSKLEIK |

### CD38 Binding Domains

In some embodiments, disclosed herein is a multispecific antibody that comprises a CD38 binding domain. In some embodiments, the CD38 binding domain comprises an antibody or antigen binding fragment or variant thereof. In some embodiments, antibody or antigen binding fragment or variant thereof is a monoclonal antibody. In some embodiments, antibody or antigen binding fragment or variant thereof is a human antibody, a murine antibody, a humanized antibody, or a chimeric antibody. In some embodiments, the CD38 binding domain comprises a monovalent Fab, a bivalent Fab′2, a single-chain variable fragment (scFv), or functional fragment or variant thereof. In some embodiments, the CD38 binding domain comprises an immunoglobulin variable heavy chain domain (VH). In some embodiments, the CD38 binding domain comprises an immunoglobulin variable light chain domain (VL). In some embodiments, the CD38 binding domain comprises a VH and a VL.

In some embodiments, the multispecific antibodies, and binding fragments thereof, are derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

### Complementarity Determining Regions (CDRs)

In some embodiments, the CD38 binding domain comprises an immunoglobulin variable heavy chain domain (VH) that comprises at least one, two, or three complementarity determining regions (CDRs) disclosed in Table 5 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises an immunoglobulin variable light chain domain (VL) that comprises at least one, two, or three complementarity determining regions (CDRs) disclosed in Table 6 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises at least one, two, or three complementarity determining regions (CDRs) disclosed in Table 5 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises at least one, two, or three complementarity determining regions (CDRs) disclosed in Table 6 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises a CDR1 of SEQ ID NO: 9, a CDR2 of SEQ ID NO: 10, and a CDR3 of SEQ ID NO: 11.

In some embodiments, the CD38 binding domain comprises a VH that comprises a CDR1 of SEQ ID NO: 12, a CDR2 of SEQ ID NO: 13, and a CDR3 of SEQ ID NO: 14.

In some embodiments, the CD38 binding domain comprises a VH that comprises a CDR1 of SEQ ID NO: 15, a CDR2 of SEQ ID NO: 16, and a CDR3 of SEQ ID NO: 11.

In some embodiments, the CD38 binding domain comprises a VH that comprises a CDR1 of SEQ ID NO: 17, a CDR2 of SEQ ID NO: 18, and a CDR3 of SEQ ID NO: 19.

In some embodiments, the CD38 binding domain comprises a VH that comprises a CDR1 of SEQ ID NO: 208, a CDR2 of SEQ ID NO: 21, and a CDR3 of SEQ ID NO: 22.

In some embodiments, the CD38 binding domain comprises a VH that comprises a CDR1 of SEQ ID NO: 23, a CDR2 of SEQ ID NO: 24, and a CDR3 of SEQ ID NO: 25.

In some embodiments, the CD38 binding domain comprises a VH that comprises a CDR1 of SEQ ID NO: 26, a CDR2 of SEQ ID NO: 27, and a CDR3 of SEQ ID NO: 28.

In some embodiments, the CD38 binding domain comprises a VH that comprises a CDR1 of SEQ ID NO: 29, a CDR2 of SEQ ID NO: 30, and a CDR3 of SEQ ID NO: 31.

In some embodiments, the CD38 binding domain comprises a VH that comprises a CDR1 of SEQ ID NO: 32, a CDR2 of SEQ ID NO: 33, and a CDR3 of SEQ ID NO: 34.

In some embodiments, the CD38 binding domain comprises a VH that comprises a CDR1 of SEQ ID NO: 35, a CDR2 of SEQ ID NO: 36, and a CDR3 of SEQ ID NO: 37.

In some embodiments, the CD38 binding domain comprises a VH that comprises a CDR1 of SEQ ID NO: 38, a CDR2 of SEQ ID NO: 39, and a CDR3 of SEQ ID NO: 40.

In some embodiments, the CD38 binding domain comprises a VH that comprises a CDR1 of SEQ ID NO: 41, a CDR2 of SEQ ID NO: 42, and a CDR3 of SEQ ID NO: 43.

In some embodiments, the CD38 binding domain comprises a VH that comprises a CDR1 of SEQ ID NO: 44, a CDR2 of SEQ ID NO: 45, and a CDR3 of SEQ ID NO: 46.

In some embodiments, the CD38 binding domain comprises a VH that comprises a CDR1 of SEQ ID NO: 47, a CDR2 of SEQ ID NO: 48, and a CDR3 of SEQ ID NO: 49.

In some embodiments, the CD38 binding domain comprises a VH that comprises a CDR1 of SEQ ID NO: 50, a CDR2 of SEQ ID NO: 51, and a CDR3 of SEQ ID NO: 52.

In some embodiments, the CD38 binding domain comprises a VH that comprises a CDR1 of SEQ ID NO: 53, a CDR2 of SEQ ID NO: 54, and a CDR3 of SEQ ID NO: 55.

In some embodiments, the CD38 binding domain comprises a VH that comprises a CDR1 of SEQ ID NO: 56, a CDR2 of SEQ ID NO: 57, and a CDR3 of SEQ ID NO: 58.

In some embodiments, the CD38 binding domain comprises a VH that comprises a CDR1 of SEQ ID NO: 59, a CDR2 of SEQ ID NO: 60, and a CDR3 of SEQ ID NO: 61.

In some embodiments, the CD38 binding domain comprises a VH that comprises a CDR1 of SEQ ID NO: 15, a CDR2 of SEQ ID NO: 221, and a CDR3 of SEQ ID NO: 11.

In some embodiments, the CD38 binding domain comprises a VH that comprises a CDR1 of SEQ ID NO: 63, a CDR2 of SEQ ID NO: 64, and a CDR3 of SEQ ID NO: 11.

In some embodiments, the CD38 binding domain comprises a VH that comprises a CDR1 of SEQ ID NO: 65, a CDR2 of SEQ ID NO: 66, and a CDR3 of SEQ ID NO: 11.

In some embodiments, the CD38 binding domain comprises a VH that comprises a CDR1 of SEQ ID NO: 9, a CDR2 of SEQ ID NO: 10, and a CDR3 of SEQ ID NO: 11.

In some embodiments, the CD38 binding domain comprises a VH that comprises a CDR1 of SEQ ID NO: 9, a CDR2 of SEQ ID NO: 10, and a CDR3 of SEQ ID NO: 177.

In some embodiments, the CD38 binding domain comprises a VH that comprises a CDR1 of SEQ ID NO: 9, a CDR2 of SEQ ID NO: 10, and a CDR3 of SEQ ID NO: 178.

In some embodiments, the CD38 binding domain comprises a VH that comprises a CDR1 of SEQ ID NO: 9, a CDR2 of SEQ ID NO: 10, and a CDR3 of SEQ ID NO: 179.

In some embodiments, the CD38 binding domain comprises a VH that comprises a CDR1 of SEQ ID NO: 9, a CDR2 of SEQ ID NO: 10, and a CDR3 of SEQ ID NO: 180.

In some embodiments, the CD38 binding domain comprises a VH that comprises a CDR1 of SEQ ID NO: 9, a CDR2 of SEQ ID NO: 10, and a CDR3 of SEQ ID NO: 181.

In some embodiments, the CD38 binding domain comprises a VH that comprises a CDR1 of SEQ ID NO: 9, a CDR2 of SEQ ID NO: 10, and a CDR3 of SEQ ID NO: 182.

In some embodiments, the CD38 binding domain comprises a VH that comprises a CDR1 of SEQ ID NO: 9, a CDR2 of SEQ ID NO: 10, and a CDR3 of SEQ ID NO: 183.

In some embodiments, the CD38 binding domain comprises a VH that comprises a CDR1 of SEQ ID NO: 9, a CDR2 of SEQ ID NO: 10, and a CDR3 of SEQ ID NO: 184.

In some embodiments, the CD38 binding domain comprises a VH that comprises a CDR1 of SEQ ID NO: 9, a CDR2 of SEQ ID NO: 10, and a CDR3 of SEQ ID NO: 185.

In some embodiments, the CD38 binding domain comprises a VH that comprises a CDR1 of SEQ ID NO: 9, a CDR2 of SEQ ID NO: 10, and a CDR3 of SEQ ID NO: 186.

In some embodiments, the CD38 binding domain comprises a VH that comprises a CDR1 of SEQ ID NO: 9, a CDR2 of SEQ ID NO: 10, and a CDR3 of SEQ ID NO: 187.

In some embodiments, the CD38 binding domain comprises a VH that comprises a CDR1 of SEQ ID NO: 9, a CDR2 of SEQ ID NO: 10, and a CDR3 of SEQ ID NO: 219.

In some embodiments, the CD38 binding domain comprises a VH that comprises a CDR1 of SEQ ID NO: 9, a CDR2 of SEQ ID NO: 10, and a CDR3 of SEQ ID NO: 210.

In some embodiments, the CD38 binding domain comprises a VL that comprises a CDR1 of SEQ ID NO: 67, a CDR2 of SEQ ID NO: 68, and a CDR3 of SEQ ID NO: 69.

In some embodiments, the CD38 binding domain comprises a VL that comprises a CDR1 of SEQ ID NO: 70, a CDR2 of SEQ ID NO: 71, and a CDR3 of SEQ ID NO: 72.

In some embodiments, the CD38 binding domain comprises a VL that comprises a CDR1 of SEQ ID NO: 73, a CDR2 of SEQ ID NO: 68, and a CDR3 of SEQ ID NO: 74.

In some embodiments, the CD38 binding domain comprises a VL that comprises a CDR1 of SEQ ID NO: 75, a CDR2 of SEQ ID NO: 76, and a CDR3 of SEQ ID NO: 77.

In some embodiments, the CD38 binding domain comprises a VL that comprises a CDR1 of SEQ ID NO: 78, a CDR2 of SEQ ID NO: 79, and a CDR3 of SEQ ID NO: 80.

In some embodiments, the CD38 binding domain comprises a VL that comprises a CDR1 of SEQ ID NO: 81, a CDR2 of SEQ ID NO: 82, and a CDR3 of SEQ ID NO: 83.

In some embodiments, the CD38 binding domain comprises a VL that comprises a CDR1 of SEQ ID NO: 84, a CDR2 of SEQ ID NO: 85, and a CDR3 of SEQ ID NO: 86.

In some embodiments, the CD38 binding domain comprises a VL that comprises a CDR1 of SEQ ID NO: 87, a CDR2 of SEQ ID NO: 88, and a CDR3 of SEQ ID NO: 89.

In some embodiments, the CD38 binding domain comprises a VL that comprises a CDR1 of SEQ ID NO: 90, a CDR2 of SEQ ID NO: 91, and a CDR3 of SEQ ID NO: 92.

In some embodiments, the CD38 binding domain comprises a VL that comprises a CDR1 of SEQ ID NO: 93, a CDR2 of SEQ ID NO: 85, and a CDR3 of SEQ ID NO: 94.

some embodiments, the CD38 binding domain comprises a VL that comprises a CDR1 of SEQ ID NO: 93, a CDR2 of SEQ ID NO: 85, and a CDR3 of SEQ ID NO: 95.

In some embodiments, the CD38 binding domain comprises a VL that comprises a CDR1 of SEQ ID NO: 96, a CDR2 of SEQ ID NO: 97, and a CDR3 of SEQ ID NO: 98.

In some embodiments, the CD38 binding domain comprises a VL that comprises a CDR1 of SEQ ID NO: 99, a CDR2 of SEQ ID NO: 85, and a CDR3 of SEQ ID NO: 100.

In some embodiments, the CD38 binding domain comprises a VL that comprises a CDR1 of SEQ ID NO: 101, a CDR2 of SEQ ID NO: 91, and a CDR3 of SEQ ID NO: 102.

In some embodiments, the CD38 binding domain comprises a VL that comprises a CDR1 of SEQ ID NO: 103, a CDR2 of SEQ ID NO: 88, and a CDR3 of SEQ ID NO: 104.

In some embodiments, the CD38 binding domain comprises a VL that comprises a CDR1 of SEQ ID NO: 105, a CDR2 of SEQ ID NO: 106, and a CDR3 of SEQ ID NO: 107.

In some embodiments, the CD38 binding domain comprises a VL that comprises a CDR1 of SEQ ID NO: 84, a CDR2 of SEQ ID NO: 68, and a CDR3 of SEQ ID NO: 108.

In some embodiments, the CD38 binding domain comprises a VL that comprises a CDR1 of SEQ ID NO: 109, a CDR2 of SEQ ID NO: 91, and a CDR3 of SEQ ID NO: 110.

In some embodiments, the CD38 binding domain comprises a VL that comprises a CDR1 of SEQ ID NO: 111, a CDR2 of SEQ ID NO: 68, and a CDR3 of SEQ ID NO: 74.

In some embodiments, the CD38 binding domain comprises a VL that comprises a CDR1 of SEQ ID NO: 67, a CDR2 of SEQ ID NO: 68, and a CDR3 of SEQ ID NO: 74.

In some embodiments, the CD38 binding domain comprises a VL that comprises a CDR1 of SEQ ID NO: 67, a CDR2 of SEQ ID NO: 68, and a CDR3 of SEQ ID NO: 112.

In some embodiments, the CD38 binding domain comprises a VL that comprises a CDR1 of SEQ ID NO: 67, a CDR2 of SEQ ID NO: 68, and a CDR3 of SEQ ID NO: 69.

In some embodiments, the CD38 binding domain comprises a VL that comprises a CDR1 of SEQ ID NO: 67, a CDR2 of SEQ ID NO: 68, and a CDR3 of SEQ ID NO: 174.

In some embodiments, the CD38 binding domain comprises a VL that comprises a CDR1 of SEQ ID NO: 67, a CDR2 of SEQ ID NO: 68, and a CDR3 of SEQ ID NO: 113.

In some embodiments, the CD38 binding domain comprises a VL that comprises a CDR1 of SEQ ID NO: 67, a CDR2 of SEQ ID NO: 68, and a CDR3 of SEQ ID NO: 188.

In some embodiments, the CD38 binding domain comprises a VL that comprises a CDR1 of SEQ ID NO: 67, a CDR2 of SEQ ID NO: 68, and a CDR3 of SEQ ID NO: 189.

In some embodiments, the CD38 binding domain comprises a VL that comprises a CDR1 of SEQ ID NO: 67, a CDR2 of SEQ ID NO: 68, and a CDR3 of SEQ ID NO: 190.

In some embodiments, the CD38 binding domain comprises a VL that comprises a CDR1 of SEQ ID NO: 67, a CDR2 of SEQ ID NO: 68, and a CDR3 of SEQ ID NO: 191.

In some embodiments, the CD38 binding domain comprises a VL that comprises a CDR1 of SEQ ID NO: 67, a CDR2 of SEQ ID NO: 68, and a CDR3 of SEQ ID NO: 192.

In some embodiments, the CD38 binding domain comprises a VL that comprises a CDR1 of SEQ ID NO: 67, a CDR2 of SEQ ID NO: 68, and a CDR3 of SEQ ID NO: 193.

In some embodiments, the CD38 binding domain comprises a VL that comprises a CDR1 of SEQ ID NO: 67, a CDR2 of SEQ ID NO: 68, and a CDR3 of SEQ ID NO: 194.

In some embodiments, the CD38 binding domain comprises a VL that comprises a CDR1 of SEQ ID NO: 67, a CDR2 of SEQ ID NO: 68, and a CDR3 of SEQ ID NO: 195.

In some embodiments, the CD38 binding domain comprises a VL that comprises a CDR1 of SEQ ID NO: 67, a CDR2 of SEQ ID NO: 68, and a CDR3 of SEQ ID NO:196.

In some embodiments, the CD38 binding domain comprises a VH that comprises a CDR1 of SEQ ID NO: 9, a CDR2 of SEQ ID NO: 10, and a CDR3 of SEQ ID NO: 11; and a VL that comprises a CDR1 of SEQ ID NO: 67, a CDR2 of SEQ ID NO: 68, and a CDR3 of SEQ ID NO: 69.

In some embodiments, the CD38 binding domain comprises a VH that comprises a CDR1 of SEQ ID NO: 12, a CDR2 of SEQ ID NO: 13, and a CDR3 of SEQ ID NO: 14; and a VL that comprises a CDR1 of SEQ ID NO: 70, a CDR2 of SEQ ID NO: 71, and a CDR3 of SEQ ID NO: 72.

In some embodiments, the CD38 binding domain comprises a VH that comprises a CDR1 of SEQ ID NO: 15, a CDR2 of SEQ ID NO: 16, and a CDR3 of SEQ ID NO: 11, and a VL that comprises a CDR1 of SEQ ID NO: 73, a CDR2 of SEQ ID NO: 68, and a CDR3 of SEQ ID NO: 74.

In some embodiments, the CD38 binding domain comprises a VH that comprises a CDR1 of SEQ ID NO: 17, a CDR2 of SEQ ID NO: 18, and a CDR3 of SEQ ID NO: 19, and a VL that comprises a CDR1 of SEQ ID NO: 75, a CDR2 of SEQ ID NO: 76, and a CDR3 of SEQ ID NO: 77.

In some embodiments, the CD38 binding domain comprises a VH that comprises a CDR1 of SEQ ID NO: 20, a CDR2 of SEQ ID NO: 21, and a CDR3 of SEQ ID NO: 22, and a VL that comprises a CDR1 of SEQ ID NO: 78, a CDR2 of SEQ ID NO: 79, and a CDR3 of SEQ ID NO: 80.

In some embodiments, the CD38 binding domain comprises a VH that comprises a CDR1 of SEQ ID NO: 23, a CDR2 of SEQ ID NO: 24, and a CDR3 of SEQ ID NO: 25, and a VL that comprises a CDR1 of SEQ ID NO: 81, a CDR2 of SEQ ID NO: 82, and a CDR3 of SEQ ID NO: 83.

In some embodiments, the CD38 binding domain comprises a VH that comprises a CDR1 of SEQ ID NO: 26, a CDR2 of SEQ ID NO: 27, and a CDR3 of SEQ ID NO: 28, and a VL that comprises a CDR1 of SEQ ID NO: 84, a CDR2 of SEQ ID NO: 85, and a CDR3 of SEQ ID NO: 86.

In some embodiments, the CD38 binding domain comprises a VH that comprises a CDR1 of SEQ ID NO: 29, a CDR2 of SEQ ID NO: 30, and a CDR3 of SEQ ID NO: 31, and a VL that comprises a CDR1 of SEQ ID NO: 87, a CDR2 of SEQ ID NO: 88, and a CDR3 of SEQ ID NO: 89.

In some embodiments, the CD38 binding domain comprises a VH that comprises a CDR1 of SEQ ID NO: 32, a CDR2 of SEQ ID NO: 33, and a CDR3 of SEQ ID NO: 34, and a VL that comprises a CDR1 of SEQ ID NO: 90, a CDR2 of SEQ ID NO: 91, and a CDR3 of SEQ ID NO: 92.

In some embodiments, the CD38 binding domain comprises a VH that comprises a CDR1 of SEQ ID NO: 35, a CDR2 of SEQ ID NO: 36, and a CDR3 of SEQ ID NO: 37, and a VL that comprises a CDR1 of SEQ ID NO: 93, a CDR2 of SEQ ID NO: 85, and a CDR3 of SEQ ID NO: 94.

In some embodiments, the CD38 binding domain comprises a VH that comprises a CDR1 of SEQ ID NO: 38, a CDR2 of SEQ ID NO: 39, and a CDR3 of SEQ ID NO: 40, and a VL that comprises a CDR1 of SEQ ID NO: 93, a CDR2 of SEQ ID NO: 95, and a CDR3 of SEQ ID NO: 95.

In some embodiments, the CD38 binding domain comprises a VH that comprises a CDR1 of SEQ ID NO: 41, a CDR2 of SEQ ID NO: 42, and a CDR3 of SEQ ID NO: 43, and a VL that comprises a CDR1 of SEQ ID NO: 96, a CDR2 of SEQ ID NO: 97, and a CDR3 of SEQ ID NO: 98.

In some embodiments, the CD38 binding domain comprises a VH that comprises a CDR1 of SEQ ID NO: 44, a CDR2 of SEQ ID NO: 45, and a CDR3 of SEQ ID NO: 46, and a VL that comprises a CDR1 of SEQ ID NO: 99, a CDR2 of SEQ ID NO: 85, and a CDR3 of SEQ ID NO: 100.

In some embodiments, the CD38 binding domain comprises a VH that comprises a CDR1 of SEQ ID NO: 47, a CDR2 of SEQ ID NO: 48, and a CDR3 of SEQ ID NO: 49, and a VL that comprises a CDR1 of SEQ ID NO: 101, a CDR2 of SEQ ID NO: 91, and a CDR3 of SEQ ID NO: 102.

In some embodiments, the CD38 binding domain comprises a VH that comprises a CDR1 of SEQ ID NO: 50, a CDR2 of SEQ ID NO: 51, and a CDR3 of SEQ ID NO: 52, and a VL that comprises a CDR1 of SEQ ID NO: 103, a CDR2 of SEQ ID NO: 88, and a CDR3 of SEQ ID NO: 104.

In some embodiments, the CD38 binding domain comprises a VH that comprises a CDR1 of SEQ ID NO: 53, a CDR2 of SEQ ID NO: 54, and a CDR3 of SEQ ID NO: 55, and a VL that comprises a CDR1 of SEQ ID NO: 105, a CDR2 of SEQ ID NO: 106, and a CDR3 of SEQ ID NO: 107.

In some embodiments, the CD38 binding domain comprises a VH that comprises a CDR1 of SEQ ID NO: 56, a CDR2 of SEQ ID NO: 57, and a CDR3 of SEQ ID NO: 58, and a VL that comprises a CDR1 of SEQ ID NO: 84, a CDR2 of SEQ ID NO: 68, and a CDR3 of SEQ ID NO: 108.

In some embodiments, the CD38 binding domain comprises a VH that comprises a CDR1 of SEQ ID NO: 59, a CDR2 of SEQ ID NO: 60, and a CDR3 of SEQ ID NO: 61, and a VL that comprises a CDR1 of SEQ ID NO: 109, a CDR2 of SEQ ID NO: 91, and a CDR3 of SEQ ID NO: 110.

TABLE 5
VH CDR amino acid sequences of anti-CD38 antibodies as defined by Kabat et al.

| | SEQ | | SEQ | | SEQ | |
|---|---|---|---|---|---|---|
| | ID | | ID | | ID | |
| Antibody | NO | CDR1 | NO | CDR2 | NO | CDR3 |
| Anti-CD38 | 9 | NYWII | 10 | AIYSPSGDIKYYADWAKG | 11 | ELSGSSYEGYFES |
| Anti- | 12 | SFAMS | 13 | AISGSGGGTYYADSVKG | 14 | DKILWFGEPVFD |
| CD38.BMK1 | | | | | | Y |
| 18E4 | 15 | NYWIC | 16 | CIYSPSGDIKYYANWAKG | 11 | ELSGSSYEGYFES |
| 17E9 | 17 | RYYVT | 18 | IIYISGTTYYATWAKG | 19 | AWPVGTYVLPL |
| 17F7 | 20 | SYYMS | 21 | FISKTAITYYASWARG | 22 | VDAYSAGDL |
| 1E2 | 23 | SYAMG | 24 | IMTSGGNIYYANWAKG | 25 | EREFYGGGTSGS |
| | | | | | | RLDL |
| 20B5 | 26 | SRYWIC | 27 | CIVAGTTNTYYASWAKG | 28 | DPRTGSNVGYFN |
| | | | | | | L |
| 21G2 | 29 | NYGVS | 30 | YILTSGGTYYANWAQG | 31 | PKDSDSSAFVSL |
| 21H9 | 32 | TYYYY | 33 | CTYTGDGATYYATWAKG | 34 | SADNSIYYGYFNL |
| | | MC | | | | |
| 22H6 | 35 | NNAIS | 36 | SIYGSGNTYYATWAKG | 37 | EGAGSSWGFNL |
| 23A2 | 38 | NNAIS | 39 | SIYGTGNTYYATWAKG | 40 | EGAGSIWGFNL |
| 23B3 | 41 | NYDMT | 42 | VISSGDNTNYARWAKG | 43 | ILYNKGRYYFTF |
| 23D1 | 44 | NIYVMC | 45 | CIGTGSGDTDYATWAKG | 46 | DPGAGTWNL |
| 25C3 | 47 | SAYDMC | 48 | CLYTVSSDSIYYASWAKG | 49 | DGDYFAL |
| 25E4 | 50 | IYTMA | 51 | IISGYGTTYYATWAKG | 52 | TTVQSTDL |
| 25F12 | 53 | SYDMS | 54 | YITYGGNIYYATWAKG | 55 | TLYTGGRYYFSL |
| 26D4 | 56 | SNDAIC | 57 | CIYAGSGNTYYASWAKG | 58 | ADTIDYFNL |
| 27F6 | 59 | SGCDMC | 60 | CIYTGSGSTYYANWAKG | 61 | DSDYLGL |
| h18E4 H1 | 15 | NYWIC | 62 | CIYSPSGDIKYYADSVKG | 11 | ELSGSSYEGYFES |
| h18E4 H2 | 15 | NYWIC | 62 | CIYSPSGDIKYYADSVKG | 11 | ELSGSSYEGYFES |
| h18E4 H3 | 15 | NYWIC | 221 | CIYSPSGDIKYYADWAKG | 11 | ELSGSSYEGYFES |
| h18E4 H4 | 63 | NYWIA | 64 | SIYSPSGDIKYYADWAKG | 11 | ELSGSSYEGYFES |
| h18E4 H5 | 65 | NYWIS | 66 | YIYSPSGDIKYYADWAKG | 11 | ELSGSSYEGYFES |
| h18E4 H6 | 9 | NYWII | 10 | AIYSPSGDIKYYADWAKG | 11 | ELSGSSYEGYFES |
| Anti-CD38 | 9 | NYWII | 10 | AIYSPSGDIKYYADWAKG | 219 | ALSGSSYEGYFES |
| HC E95A | | | | | | |
| Anti-CD38 | 9 | NYWII | 10 | AIYSPSGDIKYYADWAKG | 220 | EASGSSYEGYFES |
| HC L96A | | | | | | |
| Anti-CD38 | 9 | NYWII | 10 | AIYSPSGDIKYYADWAKG | 177 | ELAGSSYEGYFES |
| HC S97A | | | | | | |
| Anti-CD38 | 9 | NYWII | 10 | AIYSPSGDIKYYADWAKG | 178 | ELSASSYEGYFES |
| HC G98A | | | | | | |
| Anti-CD38 | 9 | NYWII | 10 | AIYSPSGDIKYYADWAKG | 179 | ELSGASYEGYFES |
| HC S99A | | | | | | |
| Anti-CD38 | 9 | NYWII | 10 | AIYSPSGDIKYYADWAKG | 180 | ELSGSAYEGYFES |
| HC S100A | | | | | | |
| Anti-CD38 | 9 | NYWII | 10 | AIYSPSGDIKYYADWAKG | 181 | ELSGSSAEGYFES |
| HCY_100a | | | | | | |
| A | | | | | | |
| Anti-CD38 | 9 | NYWII | 10 | AIYSPSGDIKYYADWAKG | 182 | ELSGSSYAGYFES |
| HCE_100b | | | | | | |
| A | | | | | | |
| Anti-CD38 | 9 | NYWII | 10 | AIYSPSGDIKYYADWAKG | 183 | ELSGSSYEAYFES |
| HCG_100c | | | | | | |
| A | | | | | | |
| Anti-CD38 | 9 | NYWII | 10 | AIYSPSGDIKYYADWAKG | 184 | ELSGSSYEGAFES |
| HCY_100d | | | | | | |
| A | | | | | | |
| Anti-CD38 | 9 | NYWII | 10 | AIYSPSGDIKYYADWAKG | 185 | ELSGSSYEGYAES |
| HCF_100e | | | | | | |
| A | | | | | | |
| Anti-CD38 | 9 | NYWII | 10 | AIYSPSGDIKYYADWAKG | 186 | ELSGSSYEGYFAS |
| HC E101A | | | | | | |
| Anti-CD38 | 9 | NYWII | 10 | AIYSPSGDIKYYADWAKG | 187 | ELSGSSYEGYFEA |
| HC S102 A | | | | | | |

TABLE 6
VL CDR amino acid sequences of anti-CD38 antibodies as defined by Kabat et al.

| | SEQ | | SEQ | | SEQ | |
|---|---|---|---|---|---|---|
| | ID | | ID | | ID | |
| Antibody | NO | CDR1 | NO | CDR2 | NO | CDR3 |
| Anti-CD38 | 67 | RSSQSVYGHNWLA | 68 | RASNLAS | 69 | QGYYSGGSYA |
| Anti- | 70 | RASQSVSSYLA | 71 | DASNRAT | 72 | QRSNWPPT |
| CD38.BMK1 | | | | | | |
| 18E4 | 73 | QCSQSVYGHNWLA | 68 | RASNLAS | 74 | QGYYNGGSYA |
| 17E9 | 75 | QSSQSVVNANNLS | 76 | LASTLAS | 77 | LGVYDDDGDNA |
| 17F7 | 78 | QASQSVYSDNRLS | 79 | STSSLAS | 80 | QGEFICTSADCFV |
| 1E2 | 81 | QASQSIYNFLN | 82 | YASTLAF | 83 | QQGWNSGILDNS |
| 20B5 | 84 | QASQSVYNNNYLA | 85 | SASTLAS | 86 | QAYYSGGIYA |
| 21G2 | 87 | QASESIYSNLA | 88 | KASTLAS | 89 | QANHMIVIYGNG |
| 21H9 | 90 | QASENIYSSLA | 91 | RASTLAS | 92 | QTYYGSTSTGFT |
| 22H6 | 93 | QSSESVYKNNYLS | 85 | SASTLAS | 94 | AGGYSGNING |
| 23A2 | 93 | QSSESVYKNNYLS | 85 | SASTLAS | 95 | AGGYTGNING |
| 23B3 | 96 | QSSQSIANSDELA | 97 | DASTLAP | 98 | QGTVYDSGWYAA |
| 23D1 | 99 | QASQTIGSRLA | 85 | SASTLAS | 100 | QSYYYTSTSYPNA |
| 25C3 | 101 | QASQNIGGYLS | 91 | RASTLAS | 102 | QTYYYSGSSRYWA |
| 25E4 | 103 | QASQNIYSNLA | 88 | KASTLAS | 104 | QSYYGATSSSFGYG |
| 25F12 | 105 | QSSQSIANSNEVA | 106 | DASTLAS | 107 | QGTVYDNVWYAA |
| 26D4 | 84 | QASQSVYNNNYLA | 68 | RASNLAS | 108 | QAYYRDPTTA |
| 27F6 | 109 | QASQNIGNYLA | 91 | RASTLAS | 110 | QSYYYTTDDNYRSWA |
| h18E4_L1 | 111 | RCSQSVYGHNWLA | 68 | RASNLAS | 74 | QGYYNGGSYA |
| h18E4_L2 | 111 | RCSQSVYGHNWLA | 68 | RASNLAS | 74 | QGYYNGGSYA |
| h18E4_L3 | 111 | RCSQSVYGHNWLA | 68 | RASNLAS | 74 | QGYYNGGSYA |
| h18E4_L4 | 67 | RSSQSVYGHNWLA | 68 | RASNLAS | 74 | QGYYNGGSYA |
| h18E4_L5 | 67 | RSSQSVYGHNWLA | 68 | RASNLAS | 74 | QGYYNGGSYA |
| h18E4_L6 | 67 | RSSQSVYGHNWLA | 68 | RASNLAS | 112 | QGYYQGGSYA |
| h18E4_L7 | 67 | RSSQSVYGHNWLA | 68 | RASNLAS | 69 | QGYYSGGSYA |
| h18E4_L8 | 67 | RSSQSVYGHNWLA | 68 | RASNLAS | 74 | QGYYNGGSYA |
| h18E4_L9 | 67 | RSSQSVYGHNWLA | 68 | RASNLAS | 113 | QGYYNAGSYA |
| Anti-CD38 | 67 | RSSQSVYGHNWLA | 68 | RASNLAS | 188 | AGYYSGGSYA |
| LC_Q89A | | | | | | |
| Anti-CD38 | 67 | RSSQSVYGHNWLA | 68 | RASNLAS | 189 | QAYYSGGSYA |
| LC_G90A | | | | | | |
| Anti-CD38 | 67 | RSSQSVYGHNWLA | 68 | RASNLAS | 190 | QGAYSGGSYA |
| LC_Y91A | | | | | | |
| Anti-CD38 | 67 | RSSQSVYGHNWLA | 68 | RASNLAS | 191 | QGYASGGSYA |
| LC_Y92A | | | | | | |
| Anti-CD38 | 67 | RSSQSVYGHNWLA | 68 | RASNLAS | 192 | QGYYAGGSYA |
| LC_S93A | | | | | | |
| Anti-CD38 | 67 | RSSQSVYGHNWLA | 68 | RASNLAS | 193 | QGYYSAGSYA |
| LC_G94A | | | | | | |
| Anti-CD38 | 67 | RSSQSVYGHNWLA | 68 | RASNLAS | 194 | QGYYSGASYA |
| LC_G95A | | | | | | |
| Anti-CD38 | 67 | RSSQSVYGHNWLA | 68 | RASNLAS | 195 | QGYYSGGAYA |
| LC_S95aA | | | | | | |
| Anti-CD38 | 67 | RSSQSVYGHNWLA | 68 | RASNLAS | 196 | QGYYSGGSAA |
| LC_Y96A | | | | | | |

In some embodiments, a CDR described herein comprises one, two, or three amino acid modifications. In some embodiments, said modification is a substitution, addition, or deletion. In some embodiments, a CDR described herein comprises one, two, or three conservative amino acid substitutions. In some embodiments, the one, two, or three amino acid modifications does not substantially modify binding to human CD38. In some embodiments, the one, two, or three amino acid modifications modifies binding to human CD38. In some embodiments, a VH-CDR3 and/or VL-CDR3 comprises an amino acid substitution that modifies binding to human CD38, immunogenicity, or some other feature. In some embodiments, the amino acid substitution is an alanine (A).

### Variable Heavy and Variable Light Regions

In some embodiments, the CD38 binding domain comprises a VH that comprises an amino acid sequence disclosed in Table 7 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence disclosed in Table 8 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence disclosed in Table 7 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence disclosed in Table 8 or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 114, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 115, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 116, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 117, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 118, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 119, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 120, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 121, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 122, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 123, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 124, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 125, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 126, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 127, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 128, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 129, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 130, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 131, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 132, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 133, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 134, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 135, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 136, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 197, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 198, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 199, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 200, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 201, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 202, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 203, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 204, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 205, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 206, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 207, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 208, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 209, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 137, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 138, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 139, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 140, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 141, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 142, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 143, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 144, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 145, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 146, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 147, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 148, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 149, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 150, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 151, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 152, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 153, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 154, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 155, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 156, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 157, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 158, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 159, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 160, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 161, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 162, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 210, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 211, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 212, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 213, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 214, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 215, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 216, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 217, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VL that comprises an amino acid sequence of SEQ ID NO: 218, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 114, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ 1D NO: 137, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 115, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 138, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 116, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 139, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 117, 118, 119, 120, 121, or 114, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 140, 141, 142, 143, 144, 145, 146, 147, or 137, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 122, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 148, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 123, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 149, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 124, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 150, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 125, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 151, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 126, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 152, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 127, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 153, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 128, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 154, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 129, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 155, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO:130, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 156, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 131, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 157, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 132, or a sequence substantially identical thereto (e.g., a sequence that 93%, 94%, 95%, 96%, 97%, 98%, or 99% sequence identity); and a VL that comprises an amino acid sequence of SEQ ID NO: 158, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 133, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 159, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 134, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 160, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 135, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 161, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of SEQ ID NO: 136, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of SEQ ID NO: 162, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

In some embodiments, the CD38 binding domain comprises a VH that comprises an amino acid sequence of any one of SEQ ID NO: 197-209, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity); and a VL that comprises an amino acid sequence of any one of SEQ ID NO: 210-218, or a sequence substantially identical thereto (e.g., a sequence that has at least 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity).

TABLE 7
Amino acid sequence of the anti-CD38 variable heavy chain binding domains.
CDRs according to Kabat et al. are underlined.

| | SEQ ID | |
|---|---|---|
| Name | NO | Amino Acid Sequence |
| Anti-CD38 | 114 | EVQLLESGGGLVQPGGSLRLSCAASGFSFNNYWIIWIRQAPGK |
| | | GLEWVAAIYSPSGDIKYYADWAKGRFTVSKDNSKNTVYLQM |
| | | NSLRAEDTAVYYCARELSGSSYEGYFESWGQGTLVTVSS |
| Anti- | 115 | EVQLLESGGGLVQPGGSLRLSCAVSGFTFNSFAMSWVRQAPG |
| CD38.BMK1 | | KGLEWVSAISGSGGGTYYADSVKGRFTISRDNSKNTLYLQMNS |
| | | LRAEDTAVYFCAKDKILWFGEPVFDYWGQGTLVTVSS |
| Chimeric 18E4 | 116 | QSLEESGGDLVKPGASLTLTCTASGFSFNNYWICWIRQAPGKG |
| | | LEWVACIYSPSGDIKYYANWAKGRFTVSKTSSTTVTLQMTSLT |
| | | GADTATYFCARELSGSSYEGYFESWGPGTLVTVSS |
| Humanized | 117 | EVQLLESGGGLVQPGGSLRLSCAASGFSFNNYWICWVRQAPG |
| 18E4_H1 | | KGLEWVSCIYSPSGDIKYYADSVKGRFTISRDNSKNTLYLQMN |
| | | SLRAEDTAVYYCAKELSGSSYEGYFESWGQGTLVTVSS |
| Humanized | 118 | EVQLLESGGGLVQPGGSLRLSCAASGFSFNNYWICWIRQAPGK |
| 18E4_H2 | | GLEWVACIYSPSGDIKYYADSVKGRFTVSKDNSKNTVYLQMN |
| | | SLRAEDTAVYYCARELSGSSYEGYFESWGQGTLVTVSS |
| Humanized | 119 | EVQLLESGGGLVQPGGSLRLSCAASGFSFNNYWICWIRQAPGK |
| 18E4_H3 | | GLEWVACIYSPSGDIKYYADWAKGRFTVSKDNSKNTVYLQM |
| | | NSLRAEDTAVYYCARELSGSSYEGYFESWGQGTLVTVSS |
| Humanized | 120 | EVQLLESGGGLVQPGGSLRLSCAASGFSFNNYWIAWIRQAPGK |
| 18E4_H4 | | GLEWVASIYSPSGDIKYYADWAKGRFTVSKDNSKNTVYLQMN |
| | | SLRAEDTAVYYCARELSGSSYEGYFESWGQGTLVTVSS |
| Humanized | 121 | EVQLLESGGGLVQPGGSLRLSCAASGFSFNNYWISWIRQAPGK |
| 18E4_H5 | | GLEWVAYIYSPSGDIKYYADWAKGRFTVSKDNSKNTVYLQM |
| | | NSLRAEDTAVYYCARELSGSSYEGYFESWGQGTLVTVSS |
| Humanized | 114 | EVQLLESGGGLVQPGGSLRLSCAASGFSFNNYWIIWIRQAPGK |
| 18E4_H6 | | GLEWVAAIYSPSGDIKYYADWAKGRFTVSKDNSKNTVYLQM |
| | | NSLRAEDTAVYYCARELSGSSYEGYFESWGQGTLVTVSS |
| 17E9 | 122 | QSVEESGGRLVTPGTPLTLTCTASGFSLSRYYVTWVRQAPGKG |
| | | LEWIGIIYISGTTYYATWAKGRFTISKSATTVDLRIASPTTEDTA |
| | | TYFCAAAWPVGTYVLPLWGPGTLVTVSS |
| 17F7 | 123 | QSVEESGGRLVTPGTPLTLTCTASGFSLSSYYMSWVRQAPGKG |
| | | LEWIGFISKTAITYYASWARGRFTISKTSTAVDLKITSPTTEDTA |
| | | TYFCARVDAYSAGDLWGPGTLVTVSS |
| 1E2 | 124 | QSVEESGGRLVTPGTPLTLTCTVSGIDLNSYAMGWVRQAPGK |
| | | GLKYIGIMTSGGNIYYANWAKGRFTISKTSTTVDLRITSPTTED |
| | | TATYFCAREREFYGGGTSGSRLDLWGQGTLVTVSS |
| 20B5 | 125 | QSLEESGGDLVKPGASLTLTCTASGFSFSSRYWICWVRQAPGK |
| | | GLEWLACIVAGTTNTYYASWAKGRFTISKTSSTTVTLQMTSLT |
| | | AADTATYFCAGDPRTGSNVGYFNLWGPGTLVTVSS |
| 21G2 | 126 | QSVEESGGRLVTPGTPLTLTCTVSGFSLSNYGVSWVRQAPGKG |
| | | LEWIGYILTSGGTYYANWAQGRFTISKTSTTVDLKITSPTTEDT |
| | | ATYFCGRPKDSDSSAFVSLWGPGTLVTVSS |
| 21H9 | 127 | QSLEESGGDLVKPGASLTLTCTASGFSATTYYYYMCWVRQAP |
| | | GKGLEWIACTYTGDGATYYATWAKGRFTISKTSSTTVTLQMT |
| | | SLTAADTATYFCARSADNSIYYGYFNLWGPGTLVTVSS |
| 22H6 | 128 | QSVEESGGRLVTPGTPLTLTCTVSGFSLSNNAISWVRQAPGKGL |
| | | QWIGSIYGSGNTYYATWAKGRFTVSKTSTTVDLKINSPTTEDT |
| | | ATYFCAREGAGSSWGFNLWGPGTLVTVSS |
| 23A2 | 129 | QSVEESGGRLVTPGTPLTLTCTVSGFSLNNNAISWVRQAPGKG |
| | | LQWIGSIYGTGNTYYATWAKGRFSVSKTSTTVDLKINSPTTED |
| | | TATYFCATEGAGSIWGFNLWGPGTLVTVSS |
| 23B3 | 130 | QSVEESGGRLVTPGTPLTLTCTVSGFSLSNYDMTWVRQAPGRG |
| | | LEWIGVISSGDNTNYARWAKGRFTISKTSSTTVDLKITSPTTED |
| | | TATYFCARILYNKGRYYFTFWGPGTLVTVSS |
| 23D1 | 131 | QELVESGGGLFQPGGSLALTCKASGFSLSNIYVMCWVRQAPG |
| | | KGLEWIACIGTGSGDTQYATWAKGRFTISKTSSTTVTLQMTSL |
| | | TAADTATYFCARDPGAGTWNLWGPGTLVTVSS |
| 25C3 | 132 | QEQLEESGGDLVKPGASLTLTCKASGFSFSSAYDMCWVRQAP |
| | | GKGLEWVACLYTVSSDSIYYASWAKGRFTISRTSSTTVTLQMT |
| | | SLTAADTATYFCARDGDYFALWGPGTLVTVSS |
| 25E4 | 133 | QSLEESGGRLVTPGTPLTLTCTVSGIDLSIYTMAWVRQAPGKG |
| | | LEYIGIISGYGTTYYATWAKGRFIVSKTSTTVDLKITSPTTEDTA |
| | | TYFCVRTTVQSTDLWGPGTLVTVSS |
| 25F12 | 134 | RSLEESGGRLVTPGTPLTLTCTTSGFSLSSYDMSWVRQAPGKG |
| | | LEWIGYITYGGNIYYATWAKGRFTTSKTSTTVDLKITSPTTEDT |
| | | ATYFCARTLYTGGRYYFSLWGPGTLVTVSS |
| 26D4 | 135 | QEQLEESGGDLVKPEGSLTLTCTASGFSFSNDAICWVRQAPGK |
| | | GLEWIACIYAGSGNTYYASWAKGRFSISKTSSTTVTLQMTSLT |
| | | VADTATYFCASADTIDYFNLWGPGTLVTVSS |
| 27F6 | 136 | QSLEESGGGLVKPGASLTLTCKASGFSFSSGCDMCWVRQAPG |
| | | KGLEWISCIYTGSGSTYYANWAKGRFTISRTSSTTVTLQMTSLT |
| | | AADTATYFCAGDSDYLGLWGPGTLVTVSS |
| Anti-CD38 | 197 | EVQLLESGGGLVQPGGSLRLSCAASGFSFNNYWIIWIRQAPGK |
| HC_E95A | | GLEWVAAIYSPSGDIKYYADWAKGRFTVSKDNSKNTVYLQM |
| | | NSLRAEDTAVYYCARALSGSSYEGYFESWGQGTLVTVSS |
| Anti-CD38 | 198 | EVQLLESGGGLVQPGGSLRLSCAASGFSFNNYWIIWIRQAPGK |
| HC_L96A | | GLEWVAAIYSPSGDIKYYADWAKGRFTVSKDNSKNTVYLQM |
| | | NSLRAEDTAVYYCAREASGSSYEGYFESWGQGTLVTVSS |
| Anti-CD38 | 199 | EVQLLESGGGLVQPGGSLRLSCAASGFSFNNYWIIWIRQAPGK |
| HC_S97A | | GLEWVAAIYSPSGDIKYYADWAKGRFTVSKDNSKNTVYLQM |
| | | NSLRAEDTAVYYCARELAGSSYEGYFESWGQGTLVTVSS |
| Anti-CD38 | 200 | EVQLLESGGGLVQPGGSLRLSCAASGFSFNNYWIIWIRQAPGK |
| HC_G98A | | GLEWVAAIYSPSGDIKYYADWAKGRFTVSKDNSKNTVYLQM |
| | | NSLRAEDTAVYYCARELSASSYEGYFESWGQGTLVTVSS |
| Anti-CD38 | 201 | EVQLLESGGGLVQPGGSLRLSCAASGFSFNNYWIIWIRQAPGK |
| HC_S99A | | GLEWVAAIYSPSGDIKYYADWAKGRFTVSKDNSKNTVYLQM |
| | | NSLRAEDTAVYYCARELSGASYEGYFESWGQGTLVTVSS |
| Anti-CD38 | 202 | EVQLLESGGGLVQPGGSLRLSCAASGFSFNNYWIIWIRQAPGK |
| HC_S100A | | GLEWVAAIYSPSGDIKYYADWAKGRFTVSKDNSKNTVYLQM |
| | | NSLRAEDTAVYYCARELSGSAYEGYFESWGQGTLVTVSS |
| Anti-CD38 | 203 | EVQLLESGGGLVQPGGSLRLSCAASGFSFNNYWIIWIRQAPGK |
| HC_Y100aA | | GLEWVAAIYSPSGDIKYYADWAKGRFTVSKDNSKNTVYLQM |
| | | NSLRAEDTAVYYCARELSGSSAEGYFESWGQGTLVTVSS |
| Anti-CD38 | 204 | EVQLLESGGGLVQPGGSLRLSCAASGFSFNNYWIIWIRQAPGK |
| HC_E100bA | | GLEWVAAIYSPSGDIKYYADWAKGRFTVSKDNSKNTVYLQM |
| | | NSLRAEDTAVYYCARELSGSSYAGYFESWGQGTLVTVSS |
| Anti-CD38 | 205 | EVQLLESGGGLVQPGGSLRLSCAASGFSFNNYWIIWIRQAPGK |
| HC_G100cA | | GLEWVAAIYSPSGDIKYYADWAKGRFTVSKDNSKNTVYLQM |
| | | NSLRAEDTAVYYCARELSGSSYEAYFESWGQGTLVTVSS |
| Anti-CD38 | 206 | EVQLLESGGGLVQPGGSLRLSCAASGFSFNNYWIIWIRQAPGK |
| HC_Y100dA | | GLEWVAAIYSPSGDIKYYADWAKGRFTVSKDNSKNTVYLQM |
| | | NSLRAEDTAVYYCARELSGSSYEGAFESWGQGTLVTVSS |
| Anti-CD38 | 207 | EVQLLESGGGLVQPGGSLRLSCAASGFSFNNYWIIWIRQAPGK |
| HC_F100eA | | GLEWVAAIYSPSGDIKYYADWAKGRFTVSKDNSKNTVYLQM |
| | | NSLRAEDTAVYYCARELSGSSYEGYAESWGQGTLVTVSS |
| Anti-CD38 | 208 | EVQLLESGGGLVQPGGSLRLSCAASGFSFNNYWIIWIRQAPGK |
| HC_E101A | | GLEWVAAIYSPSGDIKYYADWAKGRFTVSKDNSKNTVYLQM |
| | | NSLRAEDTAVYYCARELSGSSYEGYFASWGQGTLVTVSS |
| Anti-CD38 | 209 | EVQLLESGGGLVQPGGSLRLSCAASGFSFNNYWIIWIRQAPGK |
| HC_S102A | | GLEWVAAIYSPSGDIKYYADWAKGRFTVSKDNSKNTVYLQM |
| | | NSLRAEDTAVYYCARELSGSSYEGYFEAWGQGTLVTVSS |

TABLE 8
Amino acid sequence of the anti-CD38 variable light chain binding domains. CDRs
according to Kabat et al. are underlined.

| | SEQ ID | |
|---|---|---|
| Name | NO | Amino Acid Sequence |
| Anti-CD38 | 137 | DIQLTQSPSTLSASVGDRVTITCRSSQSVYGHNWLAWYQQKPGK |
| | | APKLLMYRASNLASGVPSRFSGSGSGTEFTLTISSVQPDDFATYYC |
| | | QGYYSGGSYAFGGGTKVEIK |
| Anti- | 138 | EIVLTQSPATLSLSPGERATLSCRASQSVSSYLAWYQQKPGQAPRL |
| CD38.BMK1 | | LIYDASNRATGIPARFSGSGSGTDFTLTISSLEPEDFAVYYCQQRSN |
| | | WPPTFGQGTKVEIK |
| Chimeric 18E4 | 139 | AQVLTQTPSSVSAAVGGTVTINCQCSQSVYGHNWLAWYQHKPG |
| | | QPPKLLMYRASNLASGVPSRFKGSGSGSQFTLTIGEVQSDDAATY |
| | | YCQGYYNGGSYAFGGGTEVVVR |
| Humanized | 140 | DIQMTQSPSTLSASVGDRVTITCRCSQSVYGHNWLAWYQQKPGK |
| 18E4_L1 | | APKLLIYRASNLASGVPSRFSGSGSGTEFTLTISSLQPDDFATYYCQ |
| | | GYYNGGSYAFGGGTKVEIK |
| Humanized | 141 | DIQLTQSPSTLSASVGDRVTITCRCSQSVYGHNWLAWYQQKPGK |
| 18E4_L2 | | APKLLIYRASNLASGVPSRFSGSGSGTEFTLTISSVQPDDFATYYCQ |
| | | GYYNGGSYAFGGGTKVEIK |
| Humanized | 142 | DIQLTQSPSTLSASVGDRVTITCRCSQSVYGHNWLAWYQQKPGK |
| 18E4_L3 | | APKLLMYRASNLASGVPSRFSGSGSGTEFTLTISSVQPDDFATYYC |
| | | QGYYNGGSYAFGGGTKVEIK |
| Humanized | 143 | DIQLTQSPSTLSASVGDRVTITCRSSQSVYGHNWLAWYQQKPGK |
| 18E4_L4 | | APKLLIYRASNLASGVPSRFSGSGSGTEFTLTISSVQPDDFATYYCQ |
| | | GYYNGGSYAFGGGTKVEIK |
| Humanized | 144 | DIQLTQSPSTLSASVGDRVTITCRSSQSVYGHNWLAWYQQKPGK |
| 18E4_L5 | | APKLLMYRASNLASGVPSRFSGSGSGTEFTLTISSVQPDDFATYYC |
| | | QGYYNGGSYAFGGGTKVEIK |
| Humanized | 145 | DIQLTQSPSTLSASVGDRVTITCRSSQSVYGHNWLAWYQQKPGK |
| 18E4_L6 | | APKLLMYRASNLASGVPSRFSGSGSGTEFTLTISSVQPDDFATYYC |
| | | QGYYQGGSYAFGGGTKVEIK |
| Humanized | 137 | DIQLTQSPSTLSASVGDRVTITCRSSQSVYGHNWLAWYQQKPGK |
| 18E4_L7 | | APKLLMYRASNLASGVPSRFSGSGSGTEFTLTISSVQPDDFATYYC |
| | | QGYYSGGSYAFGGGTKVEIK |
| Humanized | 146 | DIQLTQSPSTLSASVGDRVTITCRSSQSVYGHNWLAWYQQKPGK |
| 18E4_L8 | | APKLLMYRASNLASGVPSRFSGSGSGTEFTLTISSVQPDDAATYYC |
| | | QGYYNGGSYAFGGGTKVEIK |
| Humanized | 147 | DIQLTQSPSTLSASVGDRVTITCRSSQSVYGHNWLAWYQQKPGK |
| 18E4_L9 | | APKLLMYRASNLASGVPSRFSGSGSGTEFTLTISSVQPDDFATYYC |
| | | QGYYNAGSYAFGGGTKVEIK |
| 17E9 | 148 | AAVLTQTPSPVSAAVGGTVTIKCQSSQSVVNANNLSWYQQKPGQ |
| | | PPKLLIYLASTLASGVPSRFSGSGSGTQFTLTISSVQSDDAATYYCL |
| | | GVYDDDGDNAFGGGTEVVVK |
| 17F7 | 149 | AQVLTQTPSSVSAAVGGTVTINCQASQSVYSDNRLSWFQQKSGQ |
| | | PPKLLIYSTSSLASGVPSRFSGSGSGTQFTLTISGVQSDDAASYYCQ |
| | | GEFICTSADCFVFGGGTEVVVK |
| 1E2 | 150 | AYDMTQTPASVEAAVGGTVTIKCQASQSIYNFLNWYQQKPGQPP |
| | | KLLIYYASTLAFGVPSRFKGSGSGTEYTLTISGVESADAATYYCQQ |
| | | GWNSGILDNSFGGGTEVVVK |
| 20B5 | 151 | AQVLTQTPSPVSAAVGGTVTINCQASQSVYNNNYLAWFQQKPGQ |
| | | PPKLLIYSASTLASGVPSRFKGSGSGTQFTLTISEVQSDDAATYYC |
| | | QAYYSGGIYAFGGGTEVVVK |
| 21G2 | 152 | ADVVMTQTPASVSEPVGGTVTINCQASESIYSNLAWYQQKPGQPP |
| | | KLLIYKASTLASGVSSRFKGSGSGTEFTLTISDLESADAATYYCQA |
| | | NHMIVIYGNGFGGGTEVVVK |
| 21H9 | 153 | AFELTQTPFSVSEPVGGTVTINCQASENIYSSLAWYQQKPGQPPKL |
| | | LIYRASTLASGVPSRFSGSGSGTEFTLTISGVQSDDAATYYCQTYY |
| | | GSTSTGFTFGGGTEVVVK |
| 22H6 | 154 | AQVLTQTASSVSAAVGGTVTISCQSSESVYKNNYLSWYQQKPGQ |
| | | PLKCLIYSASTLASGVPSRFKGSGSGTQFTLTISDLESDDAATYYC |
| | | AGGYSGNINGFGGGTEVVVK |
| 23A2 | 155 | AQVLTQTASSVSAAVGGTVTISCQSSESVYKNNYLSWYQQKPGQ |
| | | PPKGLIYSASTLASGVPSRFKGSGSGTQFTLTISDLESDDAATYYC |
| | | AGGYTGNINGFGGGTEVVVK |
| 23B3 | 156 | AQVLTQTPSSVSAAVGGTVTINCQSSQSIANSDELAWYQQKPGQP |
| | | PKLLIYDASTLAPGVPSRFSGSGSGTQFTLTTSGVQSDDAATYYCQ |
| | | GTVYDSGWYAAFGGGTEVVVK |
| 23D1 | 157 | ADIVMTQTPSSVEAAVGGTVTIKCQASQTIGSRLAWYQQKPGQPP |
| | | KLLIYSASTLASGVSSRFKGSGSGTQFTLTISDLDSADAATYYCQS |
| | | YYYTSTSYPNAFGGGTEVVVK |
| 25C3 | 158 | ADIVMTHTPASVEAAVGGTVTIKCQASQNIGGYLSWYQQKPGQR |
| | | PKLLIYRASTLASGVPSRFKGSGSGTQFTLTISDLESADAATYYCQ |
| | | TYYYSGSSRYWAFGGGTEVVVK |
| 25E4 | 159 | ADIVMTQTPASVEAAVGGTVTINCQASQNIYSNLAWYQQKPGQR |
| | | PKLLIYKASTLASGVSSRFKGSGSGTEFTLTISDLASADAATYYCQ |
| | | SYYGATSSSFGYGFGGGTEVVVK |
| 25F12 | 160 | AQVLTQTPSSVSAVVGGTVTINCQSSQSIANSNEVAWYQQKLGQP |
| | | PKLLIYDASTLASGVPSRFSGSGSGTQFTLIISGVQSDDAATYYCQ |
| | | GTVYDNVWYAAFGGGTEVVVK |
| 26D4 | 161 | AQVLTQTPSPVSAAVGGTVTINCQASQSVYNNNYLAWYQQKPG |
| | | QPPKLLIYRASNLASGVPSRFSASGSGTQFTLTISEVQSDDAATYY |
| | | CQAYYRDPTTAFGGGTEVVVK |
| 27F6 | 162 | ADIVMTQTPSSVEAAVGGTVTIKCQASQNIGNYLAWYQQKPGQP |
| | | PKVLIYRASTLASGVPSRFKGSGSGTHFTLTISDLESADAATYYCQ |
| | | SYYYTTDDNYRSWAFGGGTEVVVK |
| Anti-CD38 | 210 | DIQLTQSPSTLSASVGDRVTITCRSSQSVYGHNWLAWYQQKPGK |
| LC_Q89A (or | | APKLLMYRASNLASGVPSRFSGSGSGTEFTLTISSVQPDDFATYYC |
| Anti-CD38_{med}) | | AGYYSGGSYAFGGGTKVEIK |
| Anti-CD38 | 211 | DIQLTQSPSTLSASVGDRVTITCRSSQSVYGHNWLAWYQQKPGK |
| LC_G90A | | APKLLMYRASNLASGVPSRFSGSGSGTEFTLTISSVQPDDFATYYC |
| | | QAYYSGGSYAFGGGTKVEIK |
| Anti-CD38 | 212 | DIQLTQSPSTLSASVGDRVTITCRSSQSVYGHNWLAWYQQKPGK |
| LC_Y91A | | APKLLMYRASNLASGVPSRFSGSGSGTEFTLTISSVQPDDFATYYC |
| | | QGAYSGGSYAFGGGTKVEIK |
| Anti-CD38 | 213 | DIQLTQSPSTLSASVGDRVTITCRSSQSVYGHNWLAWYQQKPGK |
| LC_Y92A | | APKLLMYRASNLASGVPSRFSGSGSGTEFTLTISSVQPDDFATYYC |
| | | QGYASGGSYAFGGGTKVEIK |
| Anti-CD38 | 214 | DIQLTQSPSTLSASVGDRVT1TCRSSQSVYGHNWLAWYQQKPGK |
| LC_S93A | | APKLLMYRASNLASGVPSRFSGSGSGTEFTLTISSVQPDDFATYYC |
| | | QGYYAGGSYAFGGGTKVEIK |
| Anti-CD38 | 215 | DIQLTQSPSTLSASVGDRVTITCRSSQSVYGHNWLAWYQQKPGK |
| LC_G94A (or | | APKLLMYRASNLASGVPSRFSGSGSGTEFTLTISSVQPDDFATYYC |
| Anti-CD38_{low}) | | QGYYSAGSYAFGGGTKVEIK |
| Anti-CD38 | 216 | DIQLTQSPSTLSASVGDRVTITCRSSQSVYGHNWLAWYQQKPGK |
| LC_G95A | | APKLLMYRASNLASGVPSRFSGSGSGTEFTLTISSVQPDDFATYYC |
| | | QGYYSGASYAFGGGTKVEIK |
| Anti-CD38 | 217 | DIQLTQSPSTLSASVGDRVTITCRSSQSVYGHNWLAWYQQKPGK |
| LC_S95aA | | APKLLMYRASNLASGVPSRFSGSGSGTEFTLTISSVQPDDFATYYC |
| | | QGYYSGGAYAFGGGTKVEIK |
| Anti-CD38 | 218 | DIQLTQSPSTLSASVGDRVTITCRSSQSVYGHNWLAWYQQKPGK |
| LC_Y96A | | APKLLMYRASNLASGVPSRFSGSGSGTEFTLTISSVQPDDFATYYC |
| | | QGYYSGGSAAFGGGTKVEIK |

### Multispecific Antibodies

In one aspect, provided herein are multispecific antibodies comprising a CD38 binding domain (e.g., as described herein) and an BCMA binding domain (e.g., as described herein).

In some embodiments, the multispecific antibody is bivalent (i.e. contains two antigen binding domains), trivalent, or tetravalent. In some embodiments, the multispecific antibody comprises at least two, three, or four binding domains that bind CD38. In some embodiments, each of said two, three, or four binding domains that bind CD38 can be the same or different. In some embodiments, the multispecific antibody comprises at least two, three, or four binding domains that bind BCMA. In some embodiments, each of said two, three, or four binding domains that bind BCMA can be the same or different. In some embodiments, the multispecific antibody is bispecific, trispecific, or tetraspecific.

In some embodiments, the multispecific antibody is a heterodimeric antibody.

In some embodiments, the antibody comprises four polypeptide chains, wherein a first polypeptide comprises a VH domain, a CH1 domain, a hinge, a CH2 domain, and a CH3 domain, and pairs with a second polypeptide chain that comprises a VL and a CL domain, wherein said VH and VL form a binding domain that binds CD38; and a third polypeptide comprises a VH domain, a CH1 domain, a hinge, a CH2 domain, and a CH3 domain, and pairs with a second polypeptide chain that comprises a VL and a CL domain, wherein said VH and VL form a binding domain that binds BCMA; and wherein said first polypeptide and said third polypeptide pair via one or more disulfide bond.

In some embodiments, the multispecific antibody comprises two antibody fragments connected to an antibody Fc region.

For example, in some embodiments, the antibody comprises three polypeptides, wherein the CD38 binding domain comprises a Fab, and the BCMA binding domain comprises a scFv, wherein both the Fab and scFv are connected to (either directly or indirectly via a linker) to an Fc region. In some embodiments, the antibody comprises three polypeptides, wherein the CD38 binding domain comprises a ScFv, and the BCMA binding domain comprises a Fab, wherein both the Fab and scFv are connected to (either directly or indirectly via a linker) to an Fc region.

In some embodiments, the CD38 binding domain comprises a Fab, and the BCMA binding domain comprises a Fab, wherein both the Fab and scFv are connected to (either directly or indirectly via a linker) to an Fc region. In some embodiments, the CD38 binding domain comprises a ScFv, and the BCMA binding domain comprises a ScFv, wherein both the Fab and scFv are connected to (either directly or indirectly via a linker) to an Fc region.

In some embodiments, the CD38 binding domain comprises a Fab, and the BCMA binding domain comprises a scFv, wherein both the Fab and scFv are connected to (either directly or indirectly via a linker) to the N terminus of an Fc region. In some embodiments, the CD38 binding domain comprises a ScFv, and the BCMA binding domain comprises a Fab, wherein both the Fab and scFv are connected to (either directly or indirectly via a linker) to the N terminus of an Fc region.

In some embodiments, the CD38 binding domain comprises a Fab, and the BCMA binding domain comprises a scFv, wherein the Fab is connected to (either directly or indirectly via a linker) to the N terminus of an Fc region and the ScFv is connected to (either directly or indirectly via a linker) to the C terminus ofthe Fc region. In some embodiments, the CD38 binding domain comprises a Fab, and the BCMA binding domain comprises a scFv, wherein the ScFv is connected to (either directly or indirectly via a linker) to the N terminus of an Fc region and the Fab is connected to (either directly or indirectly via a linker) to the C terminus of the Fc region.

In some embodiments, the BCMA binding domain comprises a Fab, and the CD38 binding domain comprises a scFv, wherein the Fab is connected to (either directly or indirectly via a linker) to the N terminus of an Fc region and the ScFv is connected to (either directly or indirectly via a linker) to the C terminus of the Fc region. In some embodiments, the BCMA binding domain comprises a Fab, and the CD38 binding domain comprises a scFv, wherein the ScFv is connected to (either directly or indirectly via a linker) to the N terminus of an Fc region and the Fab is connected to (either directly or indirectly via a linker) to the C terminus of the Fc region.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 163 or 224; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 164; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 165 or 166.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 167; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 168; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 169, 222, or 223.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 163; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 164; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 165.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 224; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 164; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 166.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 167; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 168; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 169.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 167; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 168; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 222.

In some embodiments, the multispecific (e.g., bispecific) antibody comprises (i) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 167; (ii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 168; and (iii) an amino acid sequence exhibiting at least 80%, 81%, 82%, 83%, 84%, 85%, 86%, 87%, 88%, 89%, 90%, 91%, 92%, 93%, 94%, 95%, 96%, 97%, 98%, or 99% sequence identity or identical to SEQ ID NO: 223.

TABLE 9
Exemplary Amino Acid Sequences of Bispecific Anti-CD38/Anti-BCMA Antibodies

| | | | | | ScFv-linker- | |
|---|---|---|---|---|---|---|
| Bispecific | | SEQ ID | | SEQ ID | Constant Heavy | SEQ ID |
| Antibody | Heavy Chain | NO: | Light Chain | NO: | Chain | NO: |
| | αCD38.BMK1 | | αCD38.BMK1 | | αBCMA.BMK2 | |
| Anti- | EVQLLESG | 163 | EIVLTQSPA | 164 | EVQLQQSGAVL | 165 |
| CD38.BMK1 | GGLVQPGG | | TLSLSPGER | | ARPGASVKMSC | |
| x Anti- | SLRLSCAV | | ATLSCRASQ | | KGSGYTFTNY | |
| BCMA.BMK2 | SGFTFNSF | | SVSSYLAW | | WMHWVKQRP | |
| | AMSWVRQ | | YQQKPGQA | | GQGLEWIGATY | |
| | APGKGLE | | PRLLIYDAS | | RGHSDTYYNQ | |
| | WVSAISGS | | NRATGIPAR | | KFKGKAKLTA | |
| | GGGTYYA | | FSGSGSGTD | | VTSTSTAYMEL | |
| | DSVKGRFT | | FTLTISSLEP | | SSLTNEDSAVY | |
| | ISRDNSKN | | EDFAVYYC | | YCTRGAIYNGY | |
| | TLYLQMNS | | QQRSNWPP | | DVLDNWGQGT | |
| | LRAEDTAV | | TFGQGTKV | | LVTVSSASTKG | |
| | YFCAKDKI | | EIKRTVAAP | | PRGSTSGGGSG | |
| | LWFGEPV | | SVFIFPPSDE | | GGSGGGGSSDI | |
| | FDYWGQG | | QLKSGTASV | | QLTQTTSSLSAS | |
| | TLVTVSSA | | VCLLNNFYP | | LGDRVTISCSAS | |
| | STKGPSVFP | | REAKVQWK | | QDISNYLNWY | |
| | LAPSSKSTS | | VDNALQSG | | QQKPDGTVELV | |
| | GGTAALGC | | NSQESVTEQ | | IYYTSNLHSGV | |
| | LVKDYFPE | | DSKDSTYSL | | PSRFSGSGSGTD | |
| | PVTVSWNS | | SSTLTLSKA | | YSLTIGYLEPED | |
| | GALTSGVH | | DYEKHKVY | | VATYYCQQYR | |
| | TFPAVLQS | | ACEVTHQG | | KLPWTFGGGS | |
| | SGLYSLSS | | LSSPVTKSF | | KLEIKRGGSGG | |
| | VVTVPSSS | | NRGEC | | SGGASKTHTCP | |
| | LGTQTYIC | | | | PCPAPELLGGPS | |
| | NVNHKPSN | | | | VFLFPPKPKDTL | |
| | TKVDKKVE | | | | MISRTPEVTCV | |
| | PKSCDKTH | | | | VVDVSHEDPEV | |
| | TCPPCPAPE | | | | KFNWYVDGVE | |
| | LLGGPSVF | | | | VHNAKTKPREE | |
| | LFPPKPKD | | | | QYNSTYRVVSV | |
| | TLMISRTPE | | | | LTVLHQDWLN | |
| | VTCVVVD | | | | GKEYKCKVSN | |
| | VSHEDPEV | | | | KALPAPIEKTIS | |
| | KFNWYVD | | | | KAKGQPREPQV | |
| | GVEVHNA | | | | YTLPPSRDELTK | |
| | KTKPREEQ | | | | NQVSLSCAVKG | |
| | YNSTYRVV | | | | FYPSDIAVEWE | |
| | SVLTVLHQ | | | | SNGQPENNYKT | |
| | DWLNGKE | | | | TPPVLDSDGSFF | |
| | YKCKVSNK | | | | LVSKLTVDKSR | |
| | ALPAPIEKT | | | | WQQGNVFSCS | |
| | ISKAKGQP | | | | VMHEALHNHY | |
| | REPQVYTL | | | | TQKSLSLSPGK | |
| | PPSRDELT | | | | | |
| | KNQVSLW | | | | | |
| | CLVKGFYP | | | | | |
| | SDIAVEWE | | | | | |
| | SNGQPENN | | | | | |
| | YKTTPPVL | | | | | |
| | DSDGSFFL | | | | | |
| | YSKLTVDK | | | | | |
| | SRWQQGN | | | | | |
| | VFSCSVMH | | | | | |
| | EALHNHYT | | | | | |
| | QKSLSLSP | | | | | |
| | GK | | | | | |
| | | | | | αBCMA.BMK2 | |
| | αCD38.BMK1 | | αCD38.BMK1 | | ADCC High | |
| Anti- | EVQLLESG | 224 | EFVLTQSPA | 164 | EVQLQQSGAVL | 166 |
| CD38.BMK1 | GGLVQPGG | | TLSLSPGER | | ARPGASVKMSC | |
| x Anti- | SLRLSCAV | | ATLSCRASQ | | KGSGYTFTNY | |
| BCMA.BMK2_ | SGFTFNSF | | SVSSYLAW | | WMHWVKQRP | |
| ADCC.high | AMSWVRQ | | YQQKPGQA | | GQGLEWIGATY | |
| | APGKGLE | | PRLLIYDAS | | RGHSDTYYNQ | |
| | WVSAISGS | | NRATGIPAR | | KFKGKAKLTA | |
| | GGGTYYA | | FSGSGSGTD | | VTSTSTAYMEL | |
| | DSVKGRFT | | FTLTISSLEP | | SSLTNEDSAVY | |
| | ISRDNSKN | | EDFAVYYC | | YCTRGAIYNGY | |
| | TLYLQMNS | | QRSNWPP | | DVLDNWGQGT | |
| | LRAEDTAV | | TFGQGTKV | | LVTVSSASTKG | |
| | YFCAKDKI | | EIKRTVAAP | | PRGSTSGGGSG | |
| | LWFGEPV | | SVFIFPPSDE | | GGSGGGGSSDI | |
| | FDYWGQG | | QLKSGTASV | | QLTQTTSSLSAS | |
| | TLVTVSSA | | VCLLNNFYP | | LGDRYTISCSAS | |
| | STKGPSVFP | | REAKVQWK | | QDISNYLNWY | |
| | LAPSSKSTS | | VDNALQSG | | QQKPDGTVELV | |
| | GGTAALGC | | NSQESVTEQ | | IYYTSNLHSGV | |
| | LVKDYFPE | | DSKDSTYSL | | PSRFSGSGSGTD | |
| | PVTVSWNS | | SSTLTLSKA | | YSLTIGYLEPED | |
| | GALTSGVH | | DYEKHKVY | | VATYYCQQYR | |
| | TFPAVLQS | | ACEVTHQG | | KLPWTFGGGS | |
| | SGLYSLSS | | LSSPVTKSF | | KLEIKRGGSGG | |
| | VVTVPSSS | | NRGEC | | SGGASKTHTCP | |
| | LGTQTYIC | | | | PCPAPELLGGP | |
| | NVNHKPSN | | | | DVFLFPPKPKD | |
| | TKVDKKVE | | | | TLMISRTPEVTC | |
| | PKSCDKTH | | | | VVVDVSHEDPE | |
| | TCPPCPAPE | | | | VKFNWYVDGV | |
| | LLGGPDVF | | | | EVHNAKTKPRE | |
| | LFPPKPKD | | | | EQYNSTYRVVS | |
| | TLMISRTPE | | | | VLTVLHQDWL | |
| | VTCVVVD | | | | NGKEYKCKVS | |
| | VSHEDPEV | | | | NKALPLPEEKTI | |
| | KFNWYVD | | | | SKAKGQPREPQ | |
| | GVEVHNA | | | | VYTLPPSRDELT | |

| | | | | | | |
|---|---|---|---|---|---|---|
| | KTKPREEQ | | | | KNQVSLSCAVK | |
| | YNSTYRVV | | | | GFYPSDIAVEW | |
| | SVLTVLHQ | | | | ESNGQPENNYK | |
| | DWLNGKE | | | | TTPPVLDSDGSF | |
| | YKCKVSNK | | | | FLVSKLTVDKS | |
| | ALPLPEEK | | | | RWQQGNVFSCS | |
| | TISKAKGQ | | | | VMHEALHNHY | |
| | PREPQVYT | | | | TQKSLSLSPGK | |
| | LPPSRDELT | | | | | |
| | KNQVSLW | | | | | |
| | CLVKGFYP | | | | | |
| | SDIAVEWE | | | | | |
| | SNGQPENN | | | | | |
| | YKTTPPVL | | | | | |
| | DSDGSFFL | | | | | |
| | YSKLTVDK | | | | | |
| | SRWQQGN | | | | | |
| | VFSCSVMH | | | | | |
| | EALHNHYT | | | | | |
| | QKSLSLSP | | | | | |
| | GK | | | | | |
| | αBCMA.BMK2 | | αBCMA.BMK2 | | αCD38 | |
| Anti-CD38 x | EVQLQQSG | 167 | DIQLTQTTS | 168 | DIQLTQSPSTLS | 169 |
| Anti- | AVLARPGA | | SLSASLGDR | | ASVGDRVTITC | |
| BCMA.BMK2 | SVKMSCKG | | VTISCSASQ | | RSSQSVYGHN | |
| | SGYTFTNY | | DISNYLNW | | WLAWYQQKPG | |
| | WMHWVK | | YQQKPDGT | | KAPKLLMYRAS | |
| | QRPGQGLE | | VELVIYYTS | | NLASGVPSRFS | |
| | WIGATYR | | NLHSGVPSR | | GSGSGTEFTLTI | |
| | GHSDTYY | | FSGSGSGTD | | SSVQPDDFATY | |
| | NQKFKGK | | YSLTIGYLE | | YCQGYYSGGS | |
| | AKLTAVTS | | PEDVATYY | | YAFGGGTKVEI | |
| | TSTAYMEL | | CQQYRKLP | | KGGGGSGGGG | |
| | SSLTNEDS | | WTFGGGSK | | SGGGGSEVQLL | |
| | AVYYCTR | | LEIKRTVAA | | ESGGGLVQPGG | |
| | GAIYNGYD | | PSVFIFPPSD | | SLRLSCAASGFS | |
| | VLDNWGQ | | EQLKSGTAS | | FNNYWIIWIRQ | |
| | GTLVTVSS | | VVCLLNNF | | APGKGLEWVA | |
| | ASTKGPSV | | YPREAKVQ | | AIYSPSGDIKY | |
| | FPLAPSSKS | | WKVDNALQ | | YADWAKGRFT | |
| | TSGGTAAL | | SGNSQESVT | | VSKDNSKNTVY | |
| | GCLVKDYF | | EQDSKDSTY | | LQMNSLRAEDT | |
| | PEPVTVSW | | SLSSTLTLS | | AVYYCARELS | |
| | NSGALTSG | | KADYEKHK | | GSSYEGYFESW | |
| | VHTFPAVL | | VYACEVTH | | GQGTLVTVSSG | |
| | QSSGLYSL | | QGLSSPVTK | | GSGGSGGASKT | |
| | SSVVTVPSS | | SFNRGEC | | HTCPPCPAPELL | |
| | SLGTQTYIC | | | | GGPSVFLFPPKP | |
| | NVNHKPSN | | | | KDTLMISRTPE | |
| | TKVDKKVE | | | | VTCVVVDVSHE | |
| | PKSCDKTH | | | | DPEVKFNWYV | |
| | TCPPCPAPE | | | | DGVEVHNAKT | |

| | | | | | | |
|---|---|---|---|---|---|---|
| | LLGGPSVF | | | | KPREEQYNSTY | |
| | LFPPKPKD | | | | RVVSVLTVLHQ | |
| | TLMISRTPE | | | | DWLNGKEYKC | |
| | VTCVVVD | | | | KVSNKALPAPIE | |
| | VSHEDPEV | | | | KTISKAKGQPR | |
| | KFNWYVD | | | | EPQVYTLPPSR | |
| | GVEVHNA | | | | DELTKNQVSLS | |
| | KTKPREEQ | | | | CAVKGFYPSDI | |
| | YNSTYRVV | | | | AVEWESNGQPE | |
| | SVLTVLHQ | | | | NNYKTTPPVLD | |
| | DWLNGKE | | | | SDGSFFLVSKLT | |
| | YKCKVSNK | | | | VDKSRWQQGN | |
| | ALPAPIEKT | | | | VFSCSVMHEAL | |
| | ISKAKGQP | | | | HNHYTQKSLSL | |
| | REPQVYTL | | | | SPGK | |
| | PPSRDELT | | | | | |
| | KNQVSLW | | | | | |
| | CLVKGFYP | | | | | |
| | SDIAVEWE | | | | | |
| | SNGQPENN | | | | | |
| | YKTTPPVL | | | | | |
| | DSDGSFFL | | | | | |
| | YSKLTVDK | | | | | |
| | SRWQQGN | | | | | |
| | VFSCSVMH | | | | | |
| | EALHNHYT | | | | | |
| | QKSLSLSP | | | | | |
| | GK | | | | | |
| CD38_{med}x | EVQLQQSG | 167 | DIQLTQTTS | 168 | DIQLTQSPSTLS | 222 |
| BCMA.BMK2 | AVLARPGA | | SLSASLGDR | | ASVGDRVTITC | |
| | SVKMSCKG | | VTISCSASQ | | RSSQSVYGHN | |
| | SGYTFTNY | | DISNYLNW | | WLAWYQQKPG | |
| | WMHWVK | | YQQKPDGT | | KAPKLLMYRAS | |
| | QRPGQGLE | | VELVIYYTS | | NLASGVPSRFS | |
| | WIGATYR | | NLHSGVPSR | | GSGSGTEFTLTI | |
| | GHSDTYY | | FSGSGSGTD | | SSVQPDDFATY | |
| | NQKFKGK | | YSLTIGYLE | | YCAGYYSGGS | |
| | AKLTAVTS | | PEDVATYY | | YAFGGGTKVEI | |
| | TSTAYMEL | | CQQYRKLP | | KGGGGSGGGG | |
| | SSLTNEDS | | WTFGGGSK | | SGGGGSEVQLL | |
| | AVYYCTR | | LEIKRTVAA | | ESGGGLVQPGG | |
| | GAIYNGYD | | PSVFIFPPSD | | SLRLSCAASGFS | |
| | VLDNWGQ | | EQLKSGTAS | | FNNYWIIWIRQ | |
| | GTLVTVSS | | VVCLLNNF | | APGKGLEWVA | |
| | ASTKGPSV | | YPREAKVQ | | AIYSPSGDIKY | |
| | FPLAPSSKS | | WKVDNALQ | | YADWAKGRFT | |
| | TSGGTAAL | | SGNSQESVT | | VSKDNSKNTVY | |
| | GCLVKDYF | | EQDSKDSTY | | LQMNSLRAEDT | |
| | PEPVTVSW | | SLSSTLTLS | | AVYYCARELS | |
| | NSGALTSG | | KADYEKHK | | GSSYEGYFESW | |
| | VHTFPAVL | | VYACEVTH | | GQGTLVTVSSG | |
| | QSSGLYSL | | QGLSSPVTK | | GSGGSGGASKT | |
| | SSVVTVPSS | | SFNRGEC | | HTCPPCPAPELL | |

| | | | | | | |
|---|---|---|---|---|---|---|
| | SLGTQTYIC | | | | GGPSVFLFPPKP | |
| | NVNHKPSN | | | | KDTLMISRTPE | |
| | TKVDKKVE | | | | VTCVVVDVSHE | |
| | PKSCDKTH | | | | DPEVKFNWYV | |
| | TCPPCPAPE | | | | DGVEVHNAKT | |
| | LLGGPSVF | | | | KPREEQYNSTY | |
| | LFPPKPKD | | | | RVVSVLTVLHQ | |
| | TLMISRTPE | | | | DWLNGKEYKC | |
| | VTCVVVD | | | | KVSNKALPAPIE | |
| | VSHEDPEV | | | | KTISKAKGQPR | |
| | KFNWYVD | | | | EPQVYTLPPSR | |
| | GVEVHNA | | | | DELTKNQVSLS | |
| | KTKPREEQ | | | | CAVKGFYPSDI | |
| | YNSTYRVV | | | | AVEWESNGQPE | |
| | SVLTVLHQ | | | | NNYKTTPPVLD | |
| | DWLNGKE | | | | SDGSFFLVSKLT | |
| | YKCKVSNK | | | | VDKSRWQQGN | |
| | ALPAPIEKT | | | | VFSCSVMHEAL | |
| | ISKAKGQP | | | | HNHYTQKSLSL | |
| | REPQVYTL | | | | SPGK | |
| | PPSRDELT | | | | | |
| | KNQVSLW | | | | | |
| | CLVKGFYP | | | | | |
| | SDIAVEWE | | | | | |
| | SNGQPENN | | | | | |
| | YKTTPPVL | | | | | |
| | DSDGSFFL | | | | | |
| | YSKLTVDK | | | | | |
| | SRWQQGN | | | | | |
| | VFSCSVMH | | | | | |
| | EALHNHYT | | | | | |
| | QKSLSLSP | | | | | |
| | GK | | | | | |
| CD38_{low}x | EVQLQQSG | 167 | DIQLTQTTS | 168 | DIQLTQSPSTLS | 223 |
| BCMA.BMK2 | AVLARPGA | | SLSASLGDR | | ASVGDRVTITC | |
| | SVKMSCKG | | VTISCSASQ | | RSSQSVYGHN | |
| | SGYTFTNY | | DISNYLNW | | WLAWYQQKPG | |
| | WMHWVK | | YQQKPDGT | | KAPKLLMYRAS | |
| | QRPGQGLE | | VELVIYYTS | | NLASGVPSRFS | |
| | WIGATYR | | NLHSGVPSR | | GSGSGTEFTLTI | |
| | GHSDTYY | | FSGSGSGTD | | SSVQPDDFATY | |
| | NQKFKGK | | YSLTIGYLE | | YCQGYYSAGS | |
| | AKLTAVTS | | PEDVATYY | | YAFGGGTKVEI | |
| | TSTAYMEL | | CQQYRKLP | | KGGGGSGGGG | |
| | SSLTNEDS | | WTFGGGSK | | SGGGGSEVQLL | |
| | AVYYCTR | | LEIKRTVAA | | ESGGGLVQPGG | |
| | GAIYNGYD | | PSVFIFPPSD | | SLRLSCAASGFS | |
| | VLDNWGQ | | EQLKSGTAS | | FNNYWIIWIRQ | |
| | GTLVTVSS | | VVCLLNNF | | APGKGLEWVA | |
| | ASTKGPSV | | YPREAKVQ | | AIYSPSGDIKY | |
| | FPLAPSSKS | | WKVDNALQ | | YADWAKGRFT | |
| | TSGGTAAL | | SGNSQESVT | | VSKDNSKNTVY | |
| | GCLVKDYF | | EQDSKDSTY | | LQMNSLRAEDT | |

| | | |
|---|---|---|
| PEPVTVSW | SLSSTLTLS | AVYYCARELS |
| NSGALTSG | KADYEKHK | GSSYEGYFESW |
| VHTFPAVL | VYACEVTH | GQGTLVTVSSG |
| QSSGLYSL | QGLSSPVTK | GSGGSGGASKT |
| SSVVTVPSS | SFNRGEC | HTCPPCPAPELL |
| SLGTQTYIC | | GGPSVFLFPPKP |
| NVNHKPSN | | KDTLMISRTPE |
| TKVDKKVE | | VTCVVVDVSHE |
| PKSCDKTH | | DPEVKFNWYV |
| TCPPCPAPE | | DGVEVHNAKT |
| LLGGPSVF | | KPREEQYNSTY |
| LFPPKPKD | | RVVSVLTVLHQ |
| TLMISRTPE | | DWLNGKEYKC |
| VTCVVVD | | KVSNKALPAPIE |
| VSHEDPEV | | KTISKAKGQPR |
| KFNWYVD | | EPQVYTLPPSR |
| GVEVHNA | | DELTKNQVSLS |
| KTKPREEQ | | CAVKGFYPSDI |
| YNSTYRVV | | AVEWESNGQPE |
| SVLTVLHQ | | NNYKTTPPVLD |
| DWLNGKE | | SDGSFFLVSKLT |
| YKCKVSNK | | VDKSRWQQGN |
| ALPAPIEKT | | VFSCSVMHEAL |
| ISKAKGQP | | HNHYTQKSLSL |
| REPQVYTL | | SPGK |
| PPSRDELT | | |
| KNQVSLW | | |
| CLVKGFYP | | |
| SDIAVEWE | | |
| SNGQPENN | | |
| YKTTPPVL | | |
| DSDGSFFL | | |
| YSKLTVDK | | |
| SRWQQGN | | |
| VFSCSVMH | | |
| EALHNHYT | | |
| QKSLSLSP | | |
| GK | | |

### Fc-Containing Multispecific Antibodies

In some embodiments, the multispecific antibodies disclosed herein include an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1 or IgG4. In some embodiments, the immunoglobulin constant region (e.g., the Fc region) is altered, e.g., mutated, to increase or decrease one or more of: Fc receptor binding, antibody glycosylation, the number of cysteine residues, effector cell function, or complement function. In other embodiments, an interface of a first and second immunoglobulin constant regions (e.g., a first and a second Fe region) is altered, e.g., mutated, to increase or decrease dimerization, e.g., relative to a non-engineered interface, e.g., a naturally-occurring interface. For example, dimerization of the immunoglobulin chain constant region (e.g., the Fc region) can be enhanced by providing an Fc interface of a first and a second Fc region with one or more of: a paired protuberance-cavity (“knob-in-a hole”), an electrostatic interaction, or a strand-exchange, such that a greater ratio of heteromultimer to homomultimer forms, e.g., relative to a non-engineered interface.

### Fc Modifications

In some embodiments, the multispecific antibody comprises at least one amino acid modification in the Fc region. A modification includes an amino acid substitution, deletion, or addition. In some embodiments, the at least one mutation comprises an amino acid substitution. In some embodiments, the at least one amino acid modification (e.g., substitution) modulates the half-life of the antibody; ability of the antibody to induce ADCC, ADCP, and/or CDC activity against a target cell; modulate stability of the antibody; and/or glycosylation of the antibody.

### Antibody Half Life

In some embodiments, the multispecific antibody comprises at least one amino acid modification that increases stability the antibody and/or increases half-life of the antibody.

In some embodiments, the antibody comprises an amino acid modification (e.g., substitution) that increases half-life of the antibody, and said modification is at one or more of positions T250, M252, 5254, T256, M428, H433, N434 of IgG1 (numbering according to EU as described in Kabat et al.), or any combination thereof. In some embodiments, the antibody comprises a T250Q and an M428L substitutions (numbering according to EU as described in Kabat et al.). In some embodiments, the antibody comprises a M252Y, S254T, and T256E substitutions (numbering according to EU as described in Kabat et al.). In some embodiments, said antibody comprises a H433K and a N434F substitution (numbering according to EU as described in Kabat et al.).

### ADCC, ADCP, and CDC

In some embodiments, the multispecific antibody comprises at least one mutation that increases ADCC. In some embodiments, the multispecific antibody comprises at least one mutation that increases antibody-dependent cellular-mediated phagocytosis (ADCP). In some embodiments, the multispecific antibody comprises at least one mutation that increases CDC.

In some embodiments, the multispecific antibody comprises a modification at least one amino acid position in the Fe region that alters an Fc receptor interaction that leads to increased ADCC. In some embodiments, the modification comprises an amino acid substitution at an amino acid position corresponding to S239, K326, A330, I332, E333 of IgG1 (EU numbering according to Kabat), or any combination thereof. In some embodiments, the multispecific antibody comprises amino acid substitutions: E333A, S239D/A330L/I332E, or K326W/E333S of IgG1 (EU numbering according to Kabat), or any combination thereof. In some embodiments, the multispecific antibody comprises amino acid substitutions S239D/A330L/I332E of IgG1 (EU numbering according to Kabat). In some embodiments, the multispecific antibody comprises amino acid substitutions: K326W/E333S of IgG1 (EU numbering according to Kabat).

In some embodiments, the human IgG constant region is modified to alter its ADCC and/or ADCP activity, e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the multispecific antibody is modified at least one amino acid position to alter interaction with an Fc receptor. Exemplary residues that modulate Fc receptor interactions include, but are not limited to, G236, S239, T250, M252, S254, T256, K326, A330, 1332, E333A, M428, H433, or N434 of IgG1 (EU numbering according to Kabat). In some instances, the mutation comprises G236A, S239D, T250Q, M252Y, S254T, T256E, K326W, A330L, I332E, E333A, E333S, M428L, H433K, or N434F of IgG1 (EU numbering according to Kabat), or any combination thereof.

### Glycosylation

In some embodiments, an amino acid corresponding to amino acid N297 in IgG1 is mutated to alter glycosylation of said antibody. In some embodiments, the Fc region is deglycosylated at residue N297.

In some embodiments, the multispecific antibody described herein has reduced or lacks glycosylation but is not modified at amino acid N297 (Kabat numbering). In these instances, the glycosylation is, for example, eliminated by production of the antibody in a host cell that lacks a post-translational glycosylation capacity, for example a bacterial or yeast derived system or a modified mammalian cell expression system. In some embodiments, the cell is a mammalian cell such as a Chinese Hamster Ovary cell line. In some embodiments, the cell is unable to express fucosyltransferase 8 (FUT8). In certain aspects, such a system is a cell-free expression system. In some embodiments, the IgG1 constant region is afucosylated. In other embodiments, the IgG1 is expressed in cells incapable of fucosylation. In some embodiments, the cell is a mammalian cell such as a Chinese Hamster Ovary cell line. In some embodiments, the cell is unable to express fucosyltransferase 8 (FUT8).

### Methods of Making Bispecific Antibodies

Various methods of producing multispecific (e.g., bispecific) antibodies have been disclosed to address the problem of incorrect heavy chain pairing. Exemplary methods are described below. Exemplary multispecific antibody formats and methods of making said multispecific antibodies are also disclosed in e.g., Speiss et al. Molecular Immunology 67 (2015) 95-106; and Klein et al mAbs 4:6, 653-663; November/December 2012; the entire contents of each of which are incorporated by reference herein.

Heterodimerized bispecific antibodies are based on the natural IgG structure, wherein the two binding arms recognize different antigens. IgG derived formats that enable defined antigen binding are generated by forced heavy chain heterodimerization, and can be combined with technologies that minimize light chain mispairing (e.g., common light chain). Forced heavy chain heterodimerization can be obtained using any number of technologies known in the art, e.g., knob-in-hole (KiH) or strand exchange engineered domains (SEED).

Any ofthe methods described or incorporated herein by reference and the associated formats can be used to make a multispecific antibody described herein.

### Knob-in-Hole

Knob-in-Hole as described in U.S. Pat. Nos. 5,731,116, 7,476,724 (the contents of each of which is incorporated herein by reference in their entirety), broadly involves: (1) mutating the CH3 domain of one or both antibodies to promote heterodimerization; and (2) combining the mutated antibodies under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, 11368A and/or Y407).

For bispecific antibodies including an Fc domain, introduction of specific mutations into the constant region of the heavy chains to promote the correct heterodimerization of the Fc portion can be utilized. Several such techniques are reviewed in Klein et al. (mAbs (2012) 4:6, 1-11), the contents of which are incorporated herein by reference in their entirety. These techniques include the “knobs-into-holes” (KiH) approach which involves the introduction of a bulky residue into one of the CH3 domains of one of the antibody heavy chains. This bulky residue fits into a complementary “hole” in the other CH3 domain of the paired heavy chain so as to promote correct pairing of heavy chains (see e.g., U.S. Pat. No. 7,642,228).

In some instances, the multispecific antibody is an IgG1, and the CH3 domain of the “knob” chain comprises a T366W mutation and the CH3 domain of the “hole” chain comprises mutations T366S, L368A, and Y407V. In some cases, the CH3 domain of the “knob” chain further comprises a Y349C mutation which forms an interchain disulfide bridge with either E356C or S354C in the CH3 domain of the “hole” chain.

In some instances, the CH3 domain of the “knob” chain comprises R409D and K370E mutations and the CH3 domain of the “hole” chain comprises D399K and E357K. In some cases, the CH3 domain of the “knob” chain further comprises a T366W mutation and the CH3 domain of the “hole” chain further comprises mutations T366S, L368A, and Y407V.

Exemplary Kill mutations include S354C, T366W in the “knob” heavy chain and Y349C, T366S, L368A, Y407V in the “hole” heavy chain. Other exemplary KiH mutations are provided in Table 10, with additional optional stabilizing Fc cysteine mutations.

TABLE 10
Exemplary Fc KiH mutations and optional Cysteine mutations

| Position (IgG1 according | | |
|---|---|---|
| to EU numbering | | |
| scheme in Kabat et al.) | Knob Mutation | Hole Mutation |
| T366 | T366W | T366S |
| L368 | — | L368A |
| Y407 | — | Y407V |
| Additional Cysteine | | |
| mutations to form | | |
| stabilizing disulfide bridge | | |
| (IgG1 according to EU | | |
| numbering scheme | | |
| in Kabat et al.) | Knob Mutation | Hole Mutation |
| S354 | S354C | — |
| Y349 | — | Y349C |

### Strand Exchange Engineered Domains (SEED)

Another heterodimeric Fc platform that supports bispecific antibody production is strand-exchange engineered domain (SEED) C(H)3 heterodimers. These derivatives of human IgG and IgA C(H)3 domains create complementary human SEED C(H)3 heterodimers that are composed of alternating segments of human IgA and IgG C(H)3 sequences. The resulting pair of SEED C(H)3 domains preferentially associates to form heterodimers when expressed in mammalian cells. SEEDbody (Sb) fusion proteins consist of [IgG1 hinge]-C(H)2-[SEED C(H)3], that may be genetically linked to one or more fusion partners (see e.g., Davis J H et al. Protein Eng Des Sel 2010; 23:195-202; and U.S. Pat. No. 8,871,912. The contents of each of which are incorporated by reference herein).

### Duobody

Another heterodimeric Fc platform that supports bispecific antibody production is the “Duobody” platform. DuoBody technology involves three basic steps to generate stable bispecific human IgG1 antibodies in a post-production exchange reaction. In a first step, two IgG1 s, each containing single matched mutations in the third constant (CH3) domain, are produced separately using standard mammalian recombinant cell lines. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (see e.g., Labrijn et al, PNAS 2013; 110(13):5145-5150 and Labrijn et al. Nature Protocols 2014; 9(10):2450-63, the contents of each of which are incorporated by reference herein).

### Electrostatic Interactions

EP1870459 and WO 2009089004 describe strategies for favoring heterodimer formation upon co-expression of different antibody domains in a host cell. In these methods, one or more residues that make up the CH3-CH3 interface in both CH3 domains are replaced with a charged amino acid such that homodimer formation is electrostatically unfavorable and heterodimerization is electrostatically favorable. Additional methods of making multispecific molecules using electrostatic interactions are described in the following references, the contents of each of which is incorporated by reference herein, include US20100015133, U.S. Pat. Nos. 8,592,562, 9,200,060, US20140154254A1, and U.S. Pat. No. 9,358,286.

### Common Light Chain

Light chain mispairing needs to be avoided to generate homogenous preparations of bispecific IgGs. One way to achieve this is through the use of the common light chain principle, i.e. combining two binders that share one light chain but still have separate specificities. An exemplary method of enhancing the formation of a desired bispecific antibody from a mixture of monomers is by providing a common variable light chain to interact with each of the heteromeric variable heavy chain regions of the bispecific antibody. Compositions and methods of producing bispecific antibodies with a common light chain as disclosed in, e.g., U.S. Pat. No. 7,183,076, US20110177073A1, EP2847231A1, WO2016079081A1, and EP3055329A1, the contents of each of which is incorporated by reference herein.

### CrossMab

Another option to reduce light chain mispairing is the CrossMab technology which avoids non-specific L chain mispairing by exchanging CH1 and CL domains in the Fab of one half of the bispecific antibody. Such crossover variants retain binding specificity and affinity, but make the two arms so different that L chain mispairing is prevented. The CrossMab technology (as reviewed in Klein et al. Supra) involves domain swapping between heavy and light chains so as to promote the formation of the correct pairings. Briefly, to construct a bispecific IgG-like CrossMab antibody that could bind to two antigens by using two distinct light chain-heavy chain pairs, a two-step modification process is applied. First, a dimerization interface is engineered into the C-terminus of each heavy chain using a heterodimerization approach, e.g., Knob-into-hole (Kill) technology, to ensure that only a heterodimer of two distinct heavy chains from one antibody (e.g., Antibody A) and a second antibody (e.g., Antibody B) is efficiently formed. Next, the constant heavy 1 (CH1) and constant light (CL) domains of one antibody are exchanged (Antibody A), keeping the variable heavy (VH) and variable light (VL) domains consistent. The exchange of the CH1 and CL domains ensured that the modified antibody (Antibody A) light chain would only efficiently dimerize with the modified antibody (antibody A) heavy chain, while the unmodified antibody (Antibody B) light chain would only efficiently dimerize with the unmodified antibody (Antibody B) heavy chain; and thus only the desired bispecific CrossMab would be efficiently formed (see e.g., Cain, C. SciBX 4(28); doi:10.1038/scibx.2011.783, the contents of which are incorporated by reference herein).

### Common Heavy Chain

An exemplary method of enhancing the formation of a desired bispecific antibody from a mixture of monomers is by providing a common variable heavy chain to interact with each of the heteromeric variable light chain regions of the bispecific antibody. Compositions and methods of producing bispecific antibodies with a common heavy chain are disclosed in, e.g., US20120184716, US20130317200, and US20160264685A1, the contents of each of which is incorporated by reference herein.

### Bispecific Antibody Binding To Target Cells

In some embodiments, a multispecific antibody of the present disclosure that comprises a CD38 binding domain and an BCMA binding domain has enhanced affinity for a cell that expresses CD38 and BCMA compared to a bivalent antibody comprising only CD38 binding domains or a bivalent antibody comprising only BCMA-binding domains.

In some embodiments, a multispecific antibody of the present disclosure that comprises a CD38 binding domain and an BCMA binding domain, binds to a cell that expresses on its surface CD38 and BCMA, with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% higher affinity (e.g., preferentially binds) compared to the binding affinity of an antibody that is bivalent to only one of CD38 or BCMA, to the cell. In some instances, the multispecific antibody has 1.5, 2, 3, 4, 5, or 10-fold higher affinity for the cell with higher BCMA expression than CD38 expression compared to a bivalent protein that binds to CD38.

In some embodiments, a multispecific antibody provided herein binds to a target cell that expresses a higher level of BCMA than CD38, on its surface. For instance, the ratio of BCMA to CD38 protein expression on the target cell surface, is from about 1, 1.5, 2.0, 2.5, 5, 10, 15, 20, 25, 50, 100, or greater than 200 as measured by flow cytometry.

In some embodiments, the target cell is a cancer cell. In some embodiments, the target cell is a B cell cancer cell. In some embodiments, the target cell is a B cell lymphoma cell. In some embodiments, the target cell is a Birkett's lymphoma cell. In some embodiments, the target cell is a myeloma cell. In some embodiments, the target cell is a multiple myeloma cell. In some embodiments, the target cell is a B cell that expresses CD38 and BCMA. In some embodiments, the B cell is a plasma cell. In some embodiments, the plasma cell is a plasmablasts, differentiated plasma cells, or long lived plasma cells. In some embodiments, the B cell secretes antibodies that recognize an autoantigen. In some embodiments, the autoantigen is associated with an autoimmune disease.

### Immunological Activity of Multispecific Antibodies on Target Cells

In some embodiments, the multispecific antibody comprising a CD38 binding domain and an BCMA binding domain has a higher immunologic activity against a CD38 expressing target cell compared to a bivalent antibody with only a CD38 binding domain or a bivalent antibody with only an BCMA binding domain. In some embodiments, the multispecific antibody has at least a 1.5, 2, 3, 4, 5, or 10-fold higher immunological activity than a bivalent antibody with only a CD38 binding domain or a bivalent antibody with only an BCMA-binding domain. In some embodiments, the multispecific antibody has a 10-20%, 21-30%, 31-40%, 41-50% or at least 51% higher immunological activity than a bivalent antibody with only a CD38 binding domain or a bivalent antibody with only an BCMA-binding domain. Various immunological activities of a multispecific antibody can be measured using in vitro assays such as an ADCC assay, an ADCP assay, and a CDC assay.

### Antibody-Dependent Cell-Mediated Cytotoxicity (ADCC)

Methods of measuring or determining ADCC activity of an antibody, including commercially available assays and kits, are well-known in the art, as described, Yamashita et al, Scientific Reports 6: article number 19772 (2016); Kantakamalakul et al, “A novel EGFP-CEM-NKr flow cytometric method for measuring antibody dependent cell mediated-cytotoxicity (ADCC) activity in HIV-1 infected individuals”, J Immunol Methods 315(Issues 1-2): 1-10; (2006); Gomez-Roman et al, “A simplified method for the rapid fluorometric assessment of antibody-dependent cell-mediated cytotoxicity”, J Immunol Methods 308 (Issues 1-2): 53-67 (2006); Schnueriger et al., Development of a quantitative, cell-line based assay to measure ADCC activity mediated by therapeutic antibodies, Molec Immunology 38 (Issues 12-13): 1512-1517 (2011); and Mata et al, “Effects of cryopreservation on effector cells for antibody dependent cell-mediated cytotoxicity (ADCC) and natural killer (NK) cell activity in ⁵¹Cr-release and CDIOVa assays J Immunol Methods 406: 1-9 (2014); all herein incorporated by reference for all purposes.

Exemplary methods of measuring or determining the ADCC activity of an antibody in the methods described herein include the ADCC assay described in the Examples or the ADCC Reporter Assay commercially available from Promega (Catalog No. G7010 and G7018).

In some instances, the multispecific antibody has a 1.5, 2, 3, 4, 5, or 10-fold higher ADCC activity than a bivalent antibody with only a CD38 binding domain (monospecific) or a bivalent antibody with only an BCMA binding domain (monospecific). In some instances, the multispecific antibody has a 10-20%, 21-30%, 31-40%, 41-50% or at least 51% higher ADCC activity than a bivalent (monospecific) antibody with a CD38 binding domain or a bivalent (monospecific) antibody with only an BCMA binding domain. ADCC activity can be determined using an in vitro cytotoxicity assay.

In some embodiments, the multispecific antibody mediates ADCC more efficiently than a bivalent antibody that comprises either the CD38 binding domain or the BCMA binding domain, wherein the ADCC activity is determined using an in vitro cytotoxicity assay. In some embodiments, the multispecific antibody mediates at least about 5%, at least about 10%, at least about 20%, at least about 30%, at least about 40%, at least about 50%, at least about 75%, at least about 100% higher maximum cytotoxicity in an in vitro ADCC assay than the bivalent antibody that comprises either the CD38 binding domain or the BCMA binding domain. In some embodiments, the multispecific antibody mediates at least 2-fold, at least 3-fold, at least 4-fold, at least 5-fold or at least 10-fold higher maximum cytotoxicity in an in vitro ADCC assay than the bivalent antibody that comprises either the CD38 binding domain or the BCMA binding domain.

### Antibody Dependent Cellular Phagocytosis (ADCP)

Suitable assays for evaluating ADCP are known in the art and include, for example, the in vitro cytotoxicity assay with macrophages as effector cells and video microscopy as described by van Bij et al. in Journal of Hepatology Volume 53, Issue 4, October 2010, Pages 677-685. A non-limiting example of an assay for determining the ADCP of CD38 expressing cells as mediated by a multispecific antibody described herein may comprise the steps of: (a) differentiating freshly isolated monocytes to macrophages with 5 days incubation in GM-CSF-containing medium; (b) plating about 100,000 macrophages per well in a multi-well plate in dendritic cell medium with GM-CSF; (c) adding 20,000 CD38-antibody opsonized CD38-expressing cells (e.g., Daudi cells), labelled with a generic fluorescent membrane dye, per well for 45 minutes at 37° C.; (d) measuring the percentage of CD14-positive, CD19-negative, membrane-dye-positive macrophages on a flow cytometer. In some embodiments, a phagocytosis assay is used to measure the ADCP effect.

In some embodiments, the multispecific antibody has a 1.5, 2, 3, 4, 5, or 10-fold higher ADCP activity than a bivalent antibody with only a CD38 binding domain (monospecific) or a bivalent antibody with only an BCMA binding domain (monospecific). In some instances, the bispecific antibody has a 10-20%, 21-30%, 31-40%, 41-50% or at least 51% higher ADCP activity than a bivalent antibody with only a CD38 binding domain or a bivalent (monospecific) antibody with only an BCMA binding domain. ADCP activity can be determined using an in vitro FACS based phagocytosis assay.

In some embodiments, the multispecific antibody mediates antibody dependent cellular phagocytosis (ADCP) more efficiently than a bivalent antibody that comprises either the CD38 binding domain or the BCMA binding domain, wherein the ADCP activity is determined using an in vitro FACS based phagocytosis assay. In some embodiments, the bispecific antibody mediates at least about 5%, at least about 10%, at least about 20%, at least about 30%, at least about 40%, at least about 50%, at least about 75%, at least about 100% higher maximum phagocytosis in an in vitro ADCP assay than the bivalent antibody that comprises either the CD38 binding domain or the BCMA binding domain.

Immunological activity can also be measured in a cell-line derived xenograft assay, wherein transformed cells are injected into mice and form a tumor. In some instances, a multispecific antibody with a CD38 binding domain and an BCMA binding domain inhibits the growth of a tumor comprising CD38 expressing cells to a greater extent than a bivalent protein with the same CD38 binding domain or a bivalent protein with the same BCMA-binding domain. In some instances, the multispecific antibody exhibits 1.5, 2, 3, 4, 5, or 10-fold higher inhibition of xenograft tumor growth compared to a bivalent protein with a CD38 binding domain or a bivalent protein with an BCMA binding domain.

### Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O′Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affmity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include grain-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells , 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Pharmaceutical Compositions, Formulations and Routes of Administration

In a further aspect, the invention provides pharmaceutical compositions comprising any of the bispecific antibodies comprising a first antigen-binding site that specifically binds to CD38 and a second antigen-binding site that specifically binds to EpCAM provided herein, e.g., for use in any of the below therapeutic methods. In one embodiment, a pharmaceutical composition comprises any of the bispecific antibodies provided herein and at least one pharmaceutically acceptable excipient. In another embodiment, a pharmaceutical composition comprises any of the bispecific antibodies provided herein and at least one additional therapeutic agent, e.g., as described below. The preparation of a pharmaceutical composition that contains at least one bispecific antibody and optionally an additional active ingredient will be known to those of skill in the art in light of the present disclosure, as exemplified by Remington's Pharmaceutical Sciences, 18th Ed. Mack Printing Company, 1990, incorporated by reference herein.

### Therapeutic Methods

Any of the antibodies described herein comprising a first antigen-binding site that specifically binds to CD38 and a second antigen-binding site that specifically binds to BCMA may be used in therapeutic methods.

For use in therapeutic methods, bispecific antibodies comprising a first antigen-binding site that specifically binds to CD38 and a second antigen-binding site that specifically binds to BCMA as defined herein before can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, bispecific antibodies comprising a first antigen-binding site that specifically binds to CD38 and a second antigen-binding site that specifically binds to BCMA as defined herein for use as a medicament are provided. In further aspects, bispecific antibodies comprising a first antigen-binding site that specifically binds to CD38 and a second antigen-binding site that specifically binds to BCMA as defined herein for use in treating a disease, in particular for use in the treatment of cancer, are provided. In certain embodiments, bispecific antibodies comprising a first antigen-binding site that specifically binds to CD38 and a second antigen-binding site that specifically binds to BCMA for use in a method of treatment are provided. In one embodiment, the invention provides bispecific antibodies comprising a first antigen-binding site that specifically binds to CD38 and a second antigen-binding site that specifically binds to BCMA as described herein for use in the treatment of a disease in an individual in need thereof. In certain embodiments, the invention provides bispecific antibodies comprising a first antigen-binding site that specifically binds to CD38 and a second antigen-binding site that specifically binds to BCMA for use in a method of treating an individual having a disease comprising administering to the individual a therapeutically effective amount of the bispecific antibody. In certain embodiments the disease to be treated is cancer. In a further aspect, the invention provides for the use of bispecific antibodies comprising a first antigen-binding site that specifically binds to CD38 and a second antigen-binding site that specifically binds to BCMA as defined herein before in the manufacture or preparation of a medicament for the treatment of a disease in an individual in need thereof. In one embodiment, the medicament is for use in a method of treating a disease comprising administering to an individual having the disease a therapeutically effective amount of the medicament.

In certain aspects, the disease to be treated is a proliferative disorder, particularly cancer. In some embodiments, the cancer is a hematological malignancy. In some embodiments, the cancer is a B cell malignancy. In some embodiments, the cancer is a B cell lymphoma. In some embodiments, the cancer is Birkett's lymphoma. In some embodiments, the cancer is a myeloma. In some embodiments, the cancer is multiple myeloma. In some embodiments, the cancer is selected from the group consisting of Birkett's lymphoma, Diffuse large B-cell lymphoma (DLBCL), Follicular lymphoma, Chronic lymphocytic leukemia (CLL), small lymphocytic lymphoma (SLL), Mantle cell lymphoma (MCL), Extranodal marginal zone B-cell lymphoma, Nodal marginal zone B-cell lymphoma, Splenic marginal zone B-cell lymphoma, Waldenstrom macroglobulinemia, and Hairy cell leukemia.

In certain aspects, the disease to be treated is an autoimmune disease. In some embodiments, the autoimmune disease is characterized by immune cells that express CD38 and BCMA. In some embodiments, the immune cells are B cells. In some embodiments, the B cells are plasma cells. In some embodiments, the plasma cells comprise plasmablasts, differentiated plasma cells, or long lived plasma cells. In some embodiments, the immune cells secrete antibodies that recognize autoantigens. In some embodiments, said autoimmune disease is selected from the group consisting of rheumatoid arthritis (RA), systemic lupus erythematosus (SLE), multiple sclerosis (MS), pemphigus vulgaris (PV), myasthenia gravis (MG) and immune thrombocytopenic purpura (ITP). In some embodiments, said autoimmune disease is rheumatoid arthritis (RA). In some embodiments, said autoimmune disease is systemic lupus erythematosus (SLE). In some embodiments, said autoimmune disease is multiple sclerosis (MS). In some embodiments, said autoimmune disease is pemphigus vulgaris (PV). In some embodiments, said autoimmune disease is myasthenia gravis (MG). In some embodiments, said autoimmune disease is immune thrombocytopenic purpura (ITP).

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antibody comprising a first antigen-binding site that specifically binds to CD38 and a second antigen-binding site that specifically binds to BCMA as defined herein before.

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### List of Embodiments

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A multispecific antibody that comprises a CD38 binding domain and a BCMA binding domain.

Embodiment 2. The multispecific antibody of embodiment 1, wherein said multispecific antibody is bispecific, trispecific, or tetraspecific.

Embodiment 3. The multispecific antibody of embodiment 2, wherein said multispecific antibody is bispecific.

Embodiment 4. The multispecific antibody of any one of embodiments 1-3, wherein said multispecific antibody is bivalent, trivalent, or tetravalent.

Embodiment 5. The multispecific antibody of embodiment 4, wherein said multispecific antibody is bivalent.

Embodiment 6. The multispecific antibody of any one of embodiments 1-5, wherein said CD38 binding domain comprises an antibody, or functional fragment or functional variant thereof, that specifically binds CD38.

Embodiment 7. The multispecific antibody of embodiment 6, wherein said antibody, or functional fragment or functional variant thereof, comprises a variable domain of an IgG heavy chain and a variable domain of an IgG light chain.

Embodiment 8. The multispecific antibody of embodiment 7, wherein said variable domain of an IgG heavy chain comprises a variable domain of an IgG1, IgG2, IgG3, or IgG4 heavy chain; and said variable domain of an IgG light chain comprises a variable domain of an IgG1, IgG2, IgG3, or IgG4 light chain.

Embodiment 9. The multispecific antibody of embodiment 8, wherein said variable domain of an IgG heavy chain comprises a variable domain of an IgG1 heavy chain; and said variable domain of an IgG light chain comprises a variable domain of an IgG1 light chain.

Embodiment 10. The multispecific antibody of any one of embodiments 6-9, wherein said antibody, or functional fragment or functional variant thereof, comprises a scFv or a Fab.

Embodiment 11. The multispecific antibody of any one of embodiments 7-10, wherein said variable domain of an IgG heavy chain (HC) of said CD38 binding domain comprises complementarity determining regions (CDRs): HC-CDR1, HC-CDR2, and HC-CDR3, wherein the HC-CDR1, the HC-CDR2, and the HC-CDR3 of said heavy chain variable domain comprise a set of sequences selected from the group consisting of the following set of sequences:

a. HC-CDR1: SEQ ID NO: 9; HC-CDR2: SEQ ID NO: 10; HC-CDR3: SEQ ID NO: 11; and

b. HC-CDR1: SEQ ID NO: 12; HC-CDR2: SEQ ID NO: 13; HC-CDR3: SEQ ID NO: 14.

Embodiment 12. The multispecific antibody of any one of embodiments 7-11, wherein said variable domain of an IgG light chain (LC) of said CD38 binding domain comprises complementarity determining regions (LC) (CDRs): LC-CDR1, LC-CDR2, and LC-CDR3, wherein the LC-CDRI, the LC-CDR2, and the LC-CDR3 of said heavy chain variable domain comprise a set of sequences selected from the group consisting of the following set of sequences:

a. LC-CDR1: SEQ ID NO: 67; LC-CDR2: SEQ ID NO: 68; LC-CDR3: SEQ ID NO: 69; and

b. LC-CDR1: SEQ ID NO: 70; LC-CDR2: SEQ ID NO: 71; LC-CDR3: SEQ ID NO: 72.

Embodiment 13. The multispecific antibody of any one of embodiments 7-12, said variable domain of an IgG light chain (LC) of said CD38 binding domain comprises complementarity determining regions (CDRs): LC-CDRI, LC-CDR2, and LC-CDR3, and said variable domain of an IgG heavy chain (HC) of said CD38 binding domain comprises CDRs: HC-CDR1, HC-CDR2, and HC-CDR3, wherein said LC: LC-CDR1, LC-CDR2, and LC-CDR3 sequences and said HC: HC-CDR1, HC-CDR2, and HC-CDR3 sequences are as set forth in any one of the following sets of sequences:

a. HC-CDR1: SEQ ID NO: 9; HC-CDR2: SEQ ID NO: 10; HC-CDR3: SEQ ID NO: 11; LC-CDRI: SEQ ID NO: 67; LC-CDR2: SEQ ID NO: 68; and LC-CDR3: SEQ ID NO: 69; or

b. HC-CDR1: SEQ ID NO: 12; HC-CDR2: SEQ ID NO: 13; HC-CDR3: SEQ ID NO: 14; and LC-CDR1: SEQ ID NO: 70; LC-CDR2: SEQ ID NO: 71; and LC-CDR3: SEQ ID NO: 72.

Embodiment 14. The multispecific antibody of any one of embodiments 7-13, wherein said variable domain of an IgG heavy chain of said CD38 binding domain comprises an amino acid sequence with at least 90%, 95%, 96%, 97%, 98%, 99%, or 100% identity to any one of SEQ ID NO: 114-136, 197-209.

Embodiment 15. The multispecific antibody of any one of embodiments 7-14, wherein said variable domain of an IgG light chain of said CD38 binding domain comprises an amino acid sequence with at least 90%, 95%, 96%, 97%, 98%, 99%, or 100% identity to any one of SEQ ID NO: 137-162, 210-218.

Embodiment 16. The multispecific antibody of any one of embodiments 7-15, wherein said CD38 binding domain comprises

a. a variable domain of an IgG heavy chain comprises an amino acid sequence with at least 90%, 95%, 96%, 97%, 98%, 99%, or 100% identity to SEQ ID NO: 114; and a variable domain of an IgG heavy chain comprises an amino acid sequence with at least 90%, 95%, 96%, 97%, 98%, 99%, or 100% identity to SEQ ID NO: 137; or

b. a variable domain of an IgG heavy chain comprises an amino acid sequence with at least 90%, 95%, 96%, 97%, 98%, 99%, or 100% identity to SEQ ID NO: 115; and a variable domain of an IgG heavy chain comprises an amino acid sequence with at least 90%, 95%, 96%, 97%, 98%, 99%, or 100% identity to SEQ ID NO: 138.

Embodiment 17. The multispecific antibody of any one of embodiments 1-16, wherein said BCMA binding domain comprises an antibody, or functional fragment or functional variant thereof, that specifically binds BCMA.

Embodiment 18. The multispecific antibody of embodiment 17, wherein said antibody, or functional fragment or functional variant thereof, comprises a variable domain of an IgG heavy chain and a variable domain of an IgG light chain.

Embodiment 19. The multispecific antibody of embodiment 18, wherein said variable domain of an IgG heavy chain comprises a variable domain of an IgG1, IgG2, IgG3, or IgG4 heavy chain; and said variable domain of an IgG light chain comprises a variable domain of an IgG1, IgG2, IgG3, or IgG4 light chain.

Embodiment 20. The multispecific antibody of embodiment 19, wherein said variable domain of an IgG heavy chain comprises a variable domain of an IgG1 heavy chain; and said variable domain of an IgG light chain comprises a variable domain of an IgG1 light chain.

Embodiment 21. The multispecific antibody of any one of embodiments 17-20, wherein said antibody, or functional fragment or functional variant thereof, comprises a scFv or a Fab.

Embodiment 22. The multispecific antibody of any one of embodiments 18-21, wherein said variable domain of an IgG heavy chain (HC) of said BCMA binding domain comprises complementarity determining regions (CDRs): HC-CDR1, HC-CDR2, and HC-CDR3, wherein said HC-CDR1, the HC-CDR2, and the HC-CDR3 of said variable heavy chain comprise a set of sequences selected from the group consisting of the following set of sequences: HC-CDR1: SEQ ID NO: 1; HC-CDR2: SEQ ID NO: 2; HC-CDR3: SEQ ID NO: 3.

Embodiment 23. The multispecific antibody of any one of embodiments 18-22, wherein said variable domain of an IgG light chain (LC) of said BCMA binding domain comprises complementarity determining regions (CDRs): LC-CDR1, LC-CDR2, and LC-CDR3, wherein the LC-CDR1, the LC-CDR2, and the LC-CDR3 of the heavy chain comprise a set of sequences selected from the group consisting of the following set of sequences: LC-CDR1: SEQ ID NO: 4; LC-CDR2: SEQ ID NO: 5; LC-CDR3: SEQ ID NO: 6.

Embodiment 24. The multispecific antibody of any one of embodiments 18-23, said variable domain of an IgG light chain (LC) of said BCMA binding domain comprises complementarity determining regions (CDRs): LC-CDR1, LC-CDR2, and LC-CDR3, and said variable domain of an IgG heavy chain (HC) of said BCMA binding domain comprises complementarity determining regions HC-CDR1, HC-CDR2, and HC-CDR3, wherein said LC: LC-CDR1, LC-CDR2, and LC-CD3 sequences and said HC: HC-CDR1, HC-CDR2, and HC-CDR3 sequences comprise sequences with at least 90%, 95%, 96%, 97%, 98%, 99%, or 100% identity to sequences as set forth in any one of the following sets of sequences: HC-CDR1: SEQ ID NO: 1; HC-CDR2: SEQ ID NO: 2; HC-CDR3: SEQ ID NO: 3; LC-CDR1: SEQ ID NO: 4; LC-CDR2: SEQ ID NO: 5; and LC-CDR3: SEQ ID NO: 6.

Embodiment 25. The multispecific antibody of any one of embodiments 18-24, wherein said variable domain of an IgG heavy chain of said BCMA binding domain comprises an amino acid sequence with at least 90%, 95%, 96%, 97%, 98%, 99%, or 100% identity to SEQ ID NO: 7.

Embodiment 26. The multispecific antibody of any one of embodiments 18-25, wherein said variable domain of an IgG light chain of said BCMA binding domain comprises an amino acid sequence with at least 90%, 95%, 96%, 97%, 98%, 99%, or 100% identity to SEQ ID NO: 8.

Embodiment 27. The multispecific antibody of any one of embodiments 18-26, wherein BCMA binding domain comprises a variable domain of an IgG heavy chain comprises an amino acid sequence with at least 90%, 95%, 96%, 97%, 98%, 99%, or 100% identity to SEQ ID NO: 7; and a variable domain of an IgG heavy chain comprises an amino acid sequence with at least 90%, 95%, 96%, 97%, 98%, 99%, or 100% identity to SEQ ID NO: 8.

Embodiment 28. The multispecific antibody of any one of embodiments 1-27, further comprising an IgG hinge region, or a portion thereof.

Embodiment 29. The multispecific antibody of any one of embodiments 1-28, further comprising an Fc region.

Embodiment 30. The multispecific antibody of embodiment 29, wherein said Fc region comprises an IgG CH2 domain and an IgG CH3 domain.

Embodiment 31. The multispecific antibody of embodiment 30, wherein said IgG hinge region is C terminal to said CD38 binding domain or said BCMA binding domain and N terminal to said Fc region.

Embodiment 32. The multispecific antibody of any one of embodiments 28-31, wherein said Fc region comprises a heterodimeric Fc region.

Embodiment 33. The multispecific antibody of any one of embodiments 28-32, wherein said Fc region comprises at least one amino acid modification that increases the half-life of the multispecific antibody.

Embodiment 34. The multispecific antibody of any one of embodiments 28-33, wherein said Fc region comprises at least one amino acid modification that modulates its interaction with an Fc receptor.

Embodiment 35. The multispecific antibody of embodiments 28-34, wherein said Fc region comprises at least one amino acid modification that increases binding of said Fc region to an Fc receptor.

Embodiment 36. The multispecific antibody of any one of embodiments 28-35, wherein said Fc region comprises at least one amino acid modification that decreases glycosylation of the Fc region.

Embodiment 37. The multispecific antibody of embodiment 36, wherein said modification is an amino acid substitution, deletion, or addition.

Embodiment 38. The multispecific antibody of embodiment 37, wherein said modification is an amino acid substitution.

Embodiment 39. The multispecific antibody of embodiment 38, wherein said at least one amino acid modification that decreases glycosylation of the Fc region comprises an amino acid substitution at a position corresponding to position N297 of human IgG1, wherein the numbering is according to the EU index of Kabat.

Embodiment 40. The multispecific antibody of any one of embodiments 28-39, wherein said Fc region is afucosylated.

Embodiment 41. The multispecific antibody of any one of embodiments 28-40, wherein said Fc region comprises at least one amino acid modification that increases antibody-dependent cellular cytotoxicity (ADCC).

Embodiment 42. The multispecific antibody of embodiment 41, wherein said modification is an amino acid substitution, deletion, or addition.

Embodiment 43. The multispecific antibody of embodiment 42, wherein said modification is an amino acid substitution.

Embodiment 44. The multispecific antibody of embodiment 43, wherein said Fc region comprises at least one mutation that increases antibody-dependent cellular cytotoxicity (ADCC), wherein said at least one mutation that increases ADCC comprises an amino acid substitution at positions corresponding to positions S239, I332, and A330 of human IgG1, wherein the amino acid numbering is according to the EU index according to Kabat et al.

Embodiment 45. The multispecific antibody of embodiment 44, wherein said amino acid substitutions are S239D, I332E, and A330L, wherein the amino acid numbering is according to the EU index according to Kabat et al.

Embodiment 46. The multispecific antibody of any one of embodiments 28-45, comprising said heterodimeric Fc region, wherein said heterodimeric Fc region comprises a knob chain and a hole chain, forming a knob-in-hole (KM) structure.

Embodiment 47. The multispecific antibody of embodiment 46, wherein said knob chain comprises an amino acid substitution at a position corresponding to T366 of IgG1, wherein amino acid position numbering is according to the EU index according to Kabat et al.

Embodiment 48. The multispecific antibody of embodiment 47, wherein said T366 substitution comprises a T336W mutation, wherein amino acid position numbering is according to the EU index according to Kabat et al.

Embodiment 49. The multispecific antibody of any one of embodiments 46-48, wherein said hole chain comprises an amino acid substitution at a position corresponding to T366, L368, or Y407 of IgG1, wherein amino acid position numbering is according to the EU index according to Kabat et al.

Embodiment 50. The multispecific antibody of embodiment 49, wherein said hole chain comprises an amino acid substitution at a position corresponding to T366, L368, and Y407 of IgG1, wherein amino acid position numbering is according to the EU index according to Kabat et al.

Embodiment 51. The multispecific antibody of embodiment 49, wherein said T366, L368, or Y407 amino acid substitutions comprise a T366S, L368A, or Y407V of IgG1, wherein amino acid position numbering is according to the EU index according to Kabat et al.

Embodiment 52. The multispecific antibody of embodiment 50, wherein said T366, L368, and Y407 amino acid substitutions comprises a T366S, L368A, and Y407V of IgG1, wherein amino acid position numbering is according to the EU index according to Kabat et al.

Embodiment 53. The multispecific antibody of any preceding embodiment, wherein said multispecific antibody has a higher affinity for CD38 expressed on the surface of a cancer cell relative to a protein that comprises said CD38 binding domain but lacks an BCMA binding domain.

Embodiment 54. The multispecific antibody of any preceding embodiment, wherein said multispecific antibody binds to a target cell that expresses CD38 and BCMA with an enhanced affinity compared to that of a protein that comprises only the first component that specifically binds to CD38 or the second component that specifically binds to BCMA.

Embodiment 55. The multispecific antibody of any preceding embodiment, wherein said multispecific antibody exhibits a higher affinity for CD38 expressed on the surface of a target cancer cell than for CD38 expressed on the surface of a non-cancer cell.

Embodiment 56. The multispecific antibody of embodiment 55, wherein BCMA is expressed on the surface of said target cancer cell, wherein said binding is measured by flow cytometry.

Embodiment 57. The multispecific antibody of any one of embodiments 1-54, wherein said multispecific antibody exhibits a higher affinity for CD38 expressed on the surface of a target plasma cell than for CD38 expressed on the surface of a non-plasma cell.

Embodiment 58. The multispecific antibody of any one of embodiments 1-54, wherein said multispecific antibody exhibits a higher affinity for CD38 expressed on the surface of a target plasma cell than for CD38 expressed on the surface of a B cell that is not a plasma cell.

Embodiment 59. The multispecific antibody of any one of embodiments 1-54, wherein said multispecific antibody exhibits a higher affinity for CD38 expressed on the surface of a target plasma cell that secretes antibodies that recognize an autoantigen than for CD38 expressed on the surface of a plasma cell that does not secrete antibodies that recognize an autoantigen.

Embodiment 60. The multispecific antibody of any one of embodiments 57-59, wherein said plasma cell is a plasmablast, differentiated plasma cell, or long lived plasma cell.

Embodiment 61. The multispecific antibody of any one of embodiments 57-58, wherein said plasma cell is a cancer cell.

Embodiment 62. The multispecific antibody of embodiment 57 or 58, wherein said plasma cell secrete antibodies that recognize autoantigens.

Embodiment 63. The multispecific antibody of any one of embodiments 57-62, wherein BCMA is expressed on the surface of said target plasma cell, wherein said binding is measured by flow cytometry.

Embodiment 64. The multispecific antibody of any preceding embodiment, wherein said multispecific antibody induces enhanced antibody-dependent cellular cytotoxicity (ADCC) activity on a target cell that expresses CD38 to BCMA compared to ADCC activity induced on said target cell by a monospecific antibody that comprises only one of the component that specifically binds to CD38 or the second component that specifically binds to BCMA.

Embodiment 65. The multispecific antibody of embodiment 64, wherein said multispecific antibody induces at least 5%, 10%, 15%, 20%, 25%, 30%, 25%, 40%, 45%, or 50% more ADCC activity than said control monospecific antibody.

Embodiment 66. The multispecific antibody of any preceding embodiment, wherein said multispecific antibody induces enhanced complement-dependent cytotoxicity (CDC) activity on a target cell that expresses CD38 to BCMA compared to CDC activity induced on said target cell by a protein that comprises only one of the component that specifically binds to CD38 or the second component that specifically binds to BCMA.

Embodiment 67. The multispecific antibody of embodiment 66, wherein said multispecific antibody induces at least 5%, 10%, 15%, 20%, 25%, 30%, 25%, 40%, 45%, or 50% more CDC activity than said control monospecific antibody.

Embodiment 68. The multispecific antibody of any one of embodiments 64-67, wherein said target cell expresses CD38 and BCMA.

Embodiment 69. The multispecific antibody of embodiment 68, wherein said target cell expresses a lower level of CD38 relative to BCMA on the surface of said target cell.

Embodiment 70. The multispecific antibody of embodiment 68 or 69, wherein the ratio of CD38 to BCMA on the surface of said cancer cells is at least about 1, 1.5, 2.0, 2.5, 5, 10, 15, 20, 50, 100, or 200.

Embodiment 71. The multispecific antibody of any one of embodiments 64-70, wherein said target cell is a cancer cell.

Embodiment 72. The multispecific antibody of embodiment 71, wherein said cancer is a hematological malignancy.

Embodiment 73. The multispecific antibody of embodiment 72, wherein said hematological malignancy is a B cell malignancy.

Embodiment 74. The multispecific antibody of embodiment 73, wherein said B cell malignancy is multiple myeloma.

Embodiment 75. The multispecific antibody of any one of embodiments 64-74, wherein said target cell is a plasma cell.

Embodiment 76. The multispecific antibody of embodiment 75, wherein said plasma cell secretes antibodies that recognize autoantigens.

Embodiment 77. The multispecific antibody of any preceding embodiment, wherein said multispecific antibody binds to a cancer cell that expresses CD38 and BCMA on the surface, and wherein the ratio of BCMA to CD38 on the surface of said cancer cell is at least about 1, 1.5, 2.0, 2.5, 5, 10, 15, 20, 50, 100, or 200.

Embodiment 78. The multispecific antibody of any preceding embodiment, wherein said multispecific antibody binds to a plasma cell that expresses CD38 and BCMA on the surface, and wherein the ratio of BCMA to CD38 on the surface of said cancer cell is at least about 1, 1.5, 2.0, 2.5, 5, 10, 15, 20, 50, 100, or 200.

Embodiment 79. The multispecific antibody of embodiment 78, wherein said plasma cell is a plasmablast, differentiated plasma cell, or long lived plasma cell.

Embodiment 80. The multispecific antibody of embodiment 78 or 79, wherein said plasma cell is a cancer cell.

Embodiment 81. The multispecific antibody of embodiment 78 or 79, wherein said plasma cell secrete antibodies that recognize autoantigens.

Embodiment 82. A nucleic acid molecule encoding the multispecific antibody of any one of embodiments 1-81.

Embodiment 83. A vector comprising the nucleic acid molecule of embodiment 82.

Embodiment 84. A pharmaceutical composition comprising the multispecific antibody of any one of embodiments 1-81.

Embodiment 85. The pharmaceutical composition of embodiment 84, further comprising a pharmaceutically acceptable carrier, an excipient, or any combinations thereof.

Embodiment 86. A method of treating a subject having cancer, said method comprising: administering to the subject the multispecific antibody of any one of embodiments 1-81 or the pharmaceutical composition of embodiment 84 or 85.

Embodiment 87. The method of embodiment 86, wherein said cancer comprises cancer cells that express CD38 and BCMA.

Embodiment 88. The method of embodiment 87, wherein the ratio of BCMA to CD38 on the surface of said cancer cells is at least about 1, 1.5, 2.0, 2.5, 5, 10, 15, 20, 50, 100, or 200.

Embodiment 89. The method of embodiment 87 or 88, wherein said cancer cells that express CD38 and BCMA are lysed.

Embodiment 90. The method of any one of embodiments 86-89, wherein multispecific antibody induces antibody-dependent cellular cytotoxicity (ADCC) mediated killing of said cancer cells that express CD38 and BCMA.

Embodiment 91. The method of any one of embodiments 86-90, wherein multispecific antibody induces complement-dependent cytotoxicity (CDC) mediated killing of said cancer cells that express CD38 and BCMA.

Embodiment 92. The method of any one of embodiments 86-91, wherein the cancer is a hematological malignancy.

Embodiment 93. The method of embodiment 92, wherein said cancer is B cell cancer.

Embodiment 94. The method of embodiment 93, wherein said cancer is multiple myeloma.

Embodiment 95. The method of any one of embodiments 86-94, comprising administering to said subject an anti-cancer agent.

Embodiment 96. The method of embodiment 95, wherein said anti-cancer agent is a chemotherapeutic agent or a biologic agent.

Embodiment 97. A method of treating a subject having an autoimmune disease, said method comprising: administering to the subject the multispecific antibody of any one of embodiments 1-81 or the pharmaceutical composition of embodiment 84 or 85.

Embodiment 98. The method of embodiment 97, wherein said autoimmune disease is characterized by immune cells that express CD38 and BCMA.

Embodiment 99. The method of embodiment 98, wherein said immune cells are B cells.

Embodiment 100. The method of embodiment 99, wherein said B cells are plasma cells.

Embodiment 101. The method of embodiment 100, wherein said plasma cells comprise plasmablasts, differentiated plasma cells, or long lived plasma cells.

Embodiment 102. The method of any one of embodiments 99-101, wherein said immune cells secrete antibodies that recognize autoantigens.

Embodiment 103. The method of any one of embodiments 97-102, wherein the ratio of BCMA to CD38 on the surface of said immune cells is at least about 1, 1.5, 2.0, 2.5, 5, 10, 15, 20, 50, 100, or 200.

Embodiment 104. The method of any one of embodiments 98-103, wherein said immune cells that express CD38 and BCMA are lysed.

Embodiment 105. The method of any one of embodiments 97-104, wherein said multispecific antibody induces antibody-dependent cellular cytotoxicity (ADCC) mediated killing of said immune cells that express CD38 and BCMA.

Embodiment 106. The method of any one of embodiments 97-105, wherein said multispecific antibody induces complement-dependent cytotoxicity (CDC) mediated killing of said immune cells that express CD38 and BCMA.

Embodiment 107. The method of any one of embodiments 97-106, wherein said autoimmune disease is selected from the group consisting of rheumatoid arthritis (RA), systemic lupus erythematosus (SLE), multiple sclerosis (MS), pemphigus vulgaris (PV), myasthenia gravis (MG) and immune thrombocytopenic purpura (ITP).

### EXAMPLES

These examples are provided for illustrative purposes only and not to limit the scope of the claims provided herein.

### Example 1

### Production of Recombinant Human CD38

The extracellular domains (ECD) of human CD38 was cloned into a pcDNA3.1 vector and transfected into Expi293 cells. The construct contained an AviTag for direct biotinylation. The amino acid sequence of the human CD38 is shown in Table 11.

TABLE 11
Amino Acid Sequence of human CD38.

| Protein | SEQ ID NO. | Amino Acid Sequence |
|---|---|---|
| hCD38 | 170 | SGLNDIFEAQKIEWHEVPRWRQQWSGPGTTKRFPETVLARCVKYTEIHP |
| | | EMRHVDCQSVWDAFKGAFISKHPCNITEEDYQPLMKLGTQTVPCNKIL |
| | | LWSRIKDLAHQFTQVQRDMFTLEDTLLGYLADDLTWCGEFNTSKINYQ |
| | | SCPDWRKDCSNNPVSVFWKTVSRRFAEAACDVVHVMLNGSRSKIFDK |
| | | NSTFGSVEVHNLQPEKVQTLEAWVIHGGREDSRDLCQDPTIKELESIISK |
| | | RNIQFSCKNIYRPDKFLQCVKNPEDSSCTSEI |

Transfected Expi293 cultures were centrifuged for 10 min at 2000 rpm and 4° C., and the supernatants were collected. Ni-NTA resin was pre-equilibrated with E buffer (137 mM NaCl, 2.7 mM KCl, 10 mM Na₂HPO₄, 2 mM KH₂PO₄, pH 7.4), incubated for 2 hours on a rotator with supernatant at 4° C., and then poured into a column. The column was washed with I-20 buffer (137 mM NaCl, 2.7 mM KCl, 10 mM Na₂HPO₄, 2 mM KH₂PO₄, pH 7.4, 20 mM imidazole) until no signal was observed by G-250. The target protein was eluted with 5 CV of I-250 buffer (137 mM NaCl, 2.7 mM KCl, 10 mM Na₂HPO₄, 2 mM KH₂PO₄, pH 7.4, 250 mM imidazole). The eluate was dialyzed against E buffer and then cleaved with TEV protease overnight at 4° C. at a 1:10 ratio of protease to protein.

For aggregation analysis, a small aliquot was loaded onto a 24 ml Superdex™ 200 column (10/300GL, GE) that had been preequilibrated with E buffer. The remainder of the protein was dialyzed into storage buffer (137 mM NaCl, 2.7 mM KCl, 10 mM Na₂HPO₄, 1.76 mM KH₂PO₄, 6% Sucrose, pH 7.4), concentrated in an ultra-filtration tube with a 30 KDa molecular weight cutoff, snap frozen with liquid N₂, and stored at −80° C.

### Example 2

### Generation and Characterization of Bispecific Anti-CD38/Anti-BCMA Antibodies

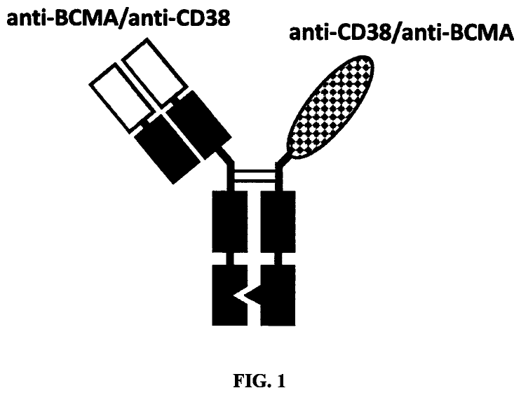

The bispecific antibody format was constructed with one CD38 binding site and one BCMA binding site in a three chain format. One antigen binding site is in a Fab format and the other antigen binding site is in a scFv format. The bispecific antibody incorporated knob-in-hole mutations in their Fc domains to form an asymmetric structure (FIG. 1).

The amino acid sequences of the bispecific antibodies used in the following experiments: Anti-CD38.BMK1×Anti-BCMA.BMK2 (SEQ ID NOS: 163-165), Anti-CD38.BMK1×Anti-BCMA.BMK2_ADCC.high (SEQ ID NOS: 224, 164, 166), Anti-CD38×Anti-BCMA.BMK2 (SEQ ID NOS: 167-169), CD38_{med}×Anti-BCMA.BMK2 (SEQ ID NOS: 167, 168, 222) and CD38_{low}×Anti-BCMA.BMK2 (SEQ ID NOS: 167, 168, 223) are disclosed in Table 9.

The amino acid sequences of the monoclonal antibodies used in the following experiments: Anti-BCMA (SEQ ID NOS: 171-172), Anti-BCMA.BMK2_ADCC.high (SEQ ID NOS: 173-174), and Anti-CD38.BMK1 (SEQ ID NOS: 175-176), are disclosed below in Table 12.

Afucosylated bispecific and monospecific antibodies were generated by transfecting the corresponding plasmids in a CHO cell line lacking FUT8 (fucosyltransferase 8) expression.

TABLE 12
Amino Acid Sequence of Exemplary Monoclonal Antibodies

| | | SEQ ID | | SEQ ID |
|---|---|---|---|---|
| Antibody | Heavy Chain | NO: | Light Chain | NO: |
| Anti-BCMA | EVQLQQSGAVLARPGA | 171 | DIQLTQTTSSLSASLGDRVTISC | 172 |
| | SVKMSCKGSGYTFTNY | | SASQDISNYLNWYQQKPDGTV | |
| | WMHWVKQRPGQGLE | | ELVIYYTSNLHSGVPSRFSGSG | |
| | WIGATYRGHSDTYYNQ | | SGTDYSLTIGYLEPEDVATYYC | |
| | KFKGKAKLTAVTSTST | | QQYRKLPWTFGGGSKLEIKRT | |
| | AYMELSSLTNEDSAVY | | VAAPSVFIFPPSDEQLKSGTASV | |
| | YCTRGAIYNGYDVLDN | | VCLLNNFYPREAKVQWKVDN | |
| | WGQGTLVTVSSASTKG | | ALQSGNSQESVTEQDSKDSTYS | |
| | PSVFPLAPSSKSTSGGTA | | LSSTLTLSKADYEKHKVYACE | |
| | ALGCLVKDYFPEPVTVS | | VTHQGLSSPVTKSFNRGEC | |
| | WNSGALTSGVHTFPAV | | | |
| | LQSSGLYSLSSVVTVPSS | | | |
| | SLGTQTYICNVNHKPSN | | | |
| | TKVDKKVEPKSCDKTH | | | |
| | TCPPCPAPELLGGPSVFL | | | |
| | FPPKPKDTLMISRTPEVT | | | |
| | CVVVDVSHEDPEVKFN | | | |
| | WYVDGVEVHNAKTKP | | | |
| | REEQYNSTYRVVSVLTV | | | |
| | LHQDWLNGKEYKCKVS | | | |
| | NKALPAPIEKTISKAKG | | | |
| | QPREPQVYTLPPSRDEL | | | |
| | TKNQVSLTCLVKGFYPS | | | |
| | DIAVEWESNGQPENNY | | | |
| | KTTPPVLDSDGSFFLYS | | | |
| | KLTVDKSRWQQGNVFS | | | |
| | CSVMHEALHNHYTQKS | | | |
| | LSLSPGK | | | |
| Anti- | EVQLQQSGAVLARPGA | 173 | DIQLTQTTSSLSASLGDRVTISC | 174 |
| BCMA. | SVKMSCKGSGYTFTNY | | SASQDISNYLNWYQQKPDGTV | |
| BMK2_ADCC. | WMHWVKQRPGQGLE | | ELVIYYTSNLHSGVPSRFSGSG | |
| high | WIGATYRGHSDTYYNQ | | SGTDYSLTIGYLEPEDVATYYC | |
| | KFKGKAKLTAVTSTST | | QQYRKLPWTFGGGSKLEIKRR | |
| | AYMELSSLTNEDSAVY | | TVAAPSVFIFPPSDEQLKSGTAS | |
| | YCTRGAIYNGYDVLDN | | VVCLLNNFYPREAKVQWKVD | |
| | WGQGTLVTVSSASTKG | | NALQSGNSQESVTEQDSKDST | |
| | PSVFPLAPSSKSTSGGTA | | YSLSSTLTLSKADYEKHKVYA | |
| | ALGCLVKDYFPEPVTVS | | CEVTHQGLSSPVTKSFNRGEC | |
| | WNSGALTSGVHTFPAV | | | |
| | LQSSGLYSLSSVVTVPSS | | | |
| | SLGTQTYICNVNHKPSN | | | |
| | TKVDKKVEPKSCDKTH | | | |
| | TCPPCPAPELLGGPDVF | | | |
| | LFPPKPKDTLMISRTPEV | | | |
| | TCVVVDVSHEDPEVKF | | | |
| | NWYVDGVEVHNAKTK | | | |
| | PREEQYNSTYRVVSVLT | | | |
| | VLHQDWLNGKEYKCK | | | |
| | VSNKALPLPEEKTISKA | | | |
| | KGQPREPQVYTLPPSRD | | | |
| | ELTKNQVSLTCLVKGFY | | | |
| | PSDIAVEWESNGQPENN | | | |
| | YKTTPPVLDSDGSFFLY | | | |
| | SKLTVDKSRWQQGNVF | | | |
| | SCSVMHEALHNHYTQK | | | |
| | SLSLSPGK | | | |
| Anti- | EVQLLESGGGLVQPGGS | 175 | EIVLTQSPATLSLSPGERATLSC | 176 |
| CD38.BMK1 | LRLSCAVSGFTFNSFAM | | RASQSVSSYLAWYQQKPGQAP | |
| | SWVRQAPGKGLEWVSA | | RLLIYDASNRATGIPARFSGSGS | |
| | ISGSGGGTYYADSVKG | | GTDFTLTISSLEPEDFAVYYCQ | |
| | RFTISRDNSKNTLYLQM | | QRSNWPPTFGQGTKVEIKRTV | |
| | NSLRAEDTAVYFCAKD | | AAPSVFIFPPSDEQLKSGTASVV | |
| | KILWFGEPVFDYWGQ | | CLLNNFYPREAKVQWKVDNA | |
| | GTLVTVSSASTKGPSVF | | LQSGNSQESVTEQDSKDSTYSL | |
| | PLAPSSKSTSGGTAALG | | SSTLTLSKADYEKHKVYACEV | |
| | CLVKDYFPEPVTVSWNS | | THQGLSSPVTKSFNRGEC | |
| | GALTSGVHTFPAVLQSS | | | |
| | GLYSLSSVVTVPSSSLGT | | | |
| | QTYICNVNHKPSNTKVD | | | |
| | KKVEPKSCDKTHTCPPC | | | |
| | PAPELLGGPSVFLFPPKP | | | |
| | KDTLMISRTPEVTCVVV | | | |
| | DVSHEDPEVKFNWYVD | | | |
| | GVEVHNAKTKPREEQY | | | |
| | NSTYRVVSVLTVLHQD | | | |
| | WLNGKEYKCKVSNKAL | | | |
| | PAPIEKTISKAKGQPREP | | | |
| | QVYTLPPSRDELTKNQV | | | |
| | SLWCLVKGFYPSDIAVE | | | |
| | WESNGQPENNYKTTPP | | | |
| | VLDSDGSFFLYSKLTVD | | | |
| | KSRWQQGNVFSCSVMH | | | |
| | EALHNHYTQKSLSLSPG | | | |

### Example 3

### ELISA Binding Assay on Bispecific CD38 a BCMA Antibodies

A 96-well plate was coated overnight at 4° C. with 1 μg/ml recombinant CD38 or BCMA (Sino Biological (10620-H03H-B)). After washing 3 times, the plate was blocked with 300 μl 1% BSA in PBST at 37° C. for 1 hour. Serially diluted antibodies were added and incubated at 37° C. for 1 hour. The plate was then washed 4 times with PBST and incubated with 1:5000 diluted 2nd antibody (Sigma, Cat #A0293) at 37° C. for 1 hour. The plate was washed again 4 times with PBST, incubated with TMB substrate for 15 min at room temperature, terminated with IN HCl, and then read at 450 nm.

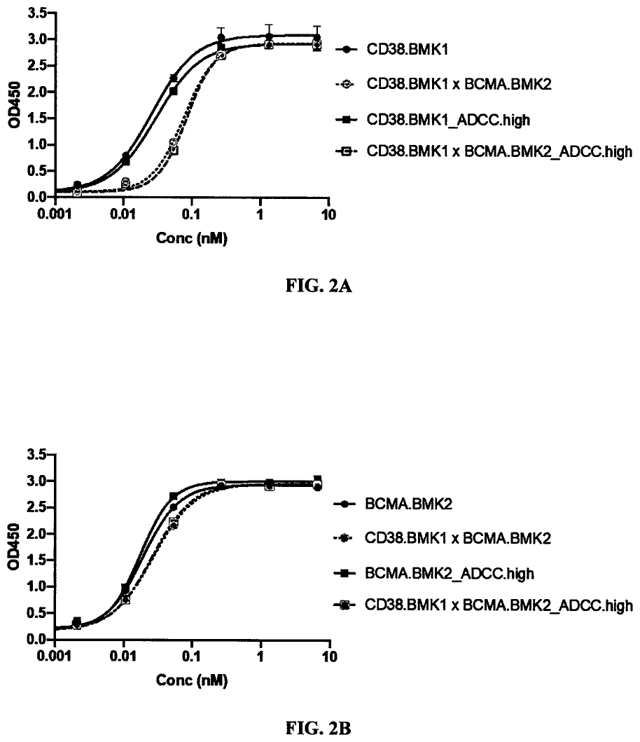

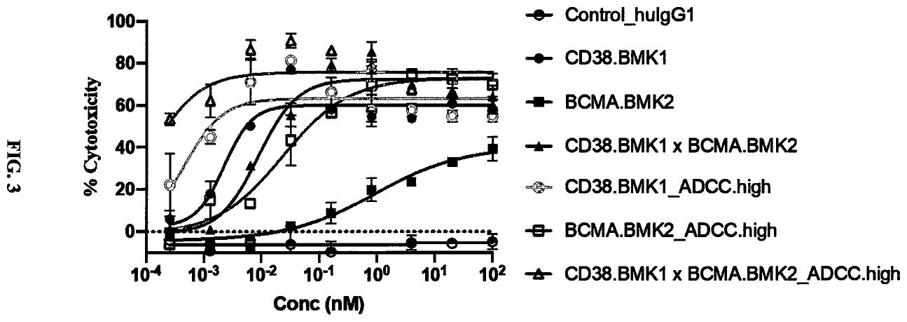

Anti-CD38 BMK1 antibody and its ADCC-high variant bound to CD38 with a EC50 of approximately 30 pM, whereas the bispecific CD38×BCMA antibodies bound with a EC50 of approximately 80 pM (FIG. 2A). Anti-BCMA BMK2 antibody and its ADCC-high variant bound to BCMA with a EC50 of approximately 20 pM, and the bispecific CD38×BCMA antibodies bound with a EC50 of approximately 30 pM (FIG. 2B).

### Example 4

### Functional Assays on Bispecific CD38×BCMA Antibodies

### Antibody-Dependent Cellular Cytotoxicity

To assay for antibody-dependent cellular cytotoxicity (ADCC), target cells were washed once with balanced salt solution or culture medium and cell numbers were adjusted to 1×10⁶ cells/ml. 2 μL of BATDA fluorescence enhancing ligand (Perkin Elmer, Cat #C136-100) was then added to each mL of cells and incubated for 20 min at 37° C. in a cell incubator. After incubation, cells were centrifuged, culture medium was aspirated. The labeled cells were washed 4 times with PBS. After the final wash, cells were re-suspended in culture medium and adjusted to 5×10⁴ cell/ml. 200 μL cell suspension was then added to each well of the 96-well plate to make the cell number per well to 1×10⁴. Background release was determined by withdrawing an aliquot of the labeled target cells, centrifuge and supernatant was transferred into an empty well. The reading was background release. 1×10⁴ labeled target cells were transferred to sterile 96-well assay plate. Antibodies were serially diluted with RPMI-1640 containing 10% FBS. 50 μL of serially-diluted antibodies were added to assay plate containing target cell and incubated at 37° C., 5% CO2 for 5-10 min. Freshly isolated PBMC were harvested and suspended in RPMI-1640 containing 10% FBS. 50 ul/well effector cells were added to each well of assay plate at different ET ratio. Set up controls: target spontaneous (target cell+100 μL medium); target maximum (target cell+100 μL medium+10 μL lysis buffer); background (100 μL the labeled target cell supernatant and 100 μL dilution medium). The plates were incubated in a humidified 5% CO₂ atmosphere at 37° C. for 2 hours. At the end of incubation, 10 μL of Lysis Buffer (Perkin Elmer, Cat #4005-0010) was added to the maximum release well. The plates were centrifuged for 5 min at 500 g. 20 μL of the supernatant from each well was transferred to a flat-bottom detection plate. 200 μL of Europium Solution (Perkin Elmer, Cat #C135-100) was then added to each well of the detection plate. The plate was shaken at 250 rpm for 15 min at room temperature and the fluorescence was then measured in a time-resolved fluorometer within 5 hrs.

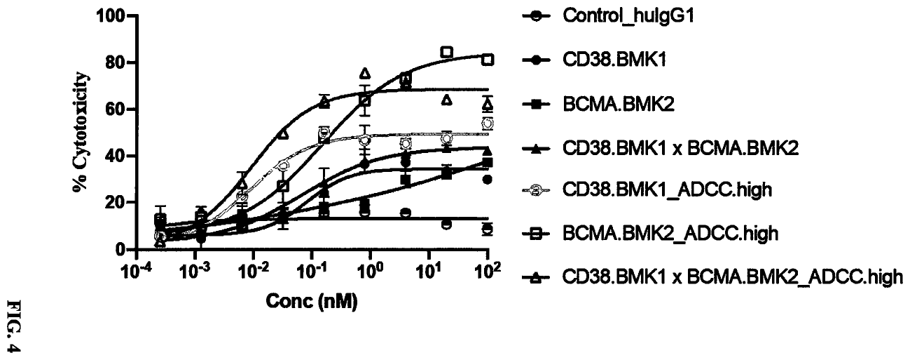

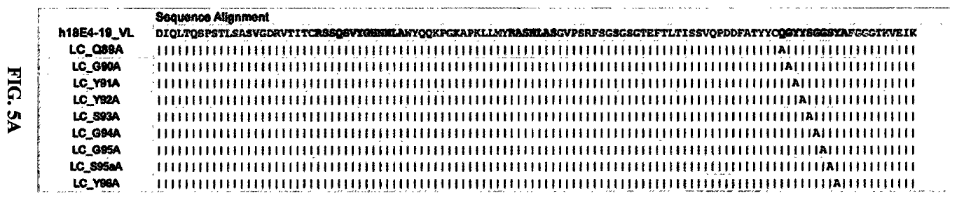

The ADCC activity of bispecific CD38×BCMA antibodies was determined on human Burkitt's lymphoma Daudi cell and human myeloma HuNS1 cell. Daudi cell expresses high level of CD38 receptors. Both the anti-CD38 antibody and the bispecific antibody showed robust ADCC activities. With ADCC high Fc mutation, all antibody variants showed improved activities and the ADCC high bispecific CD38×BCMA antibody showed superior ADCC activity (FIG. 3). Similar results were found in the myeloma cell line HuNS1. The Fc mutation improved ADCC activities for all the variants and the ADCC high bispecific CD38×BCMA antibody showed superior ADCC activity (FIG. 4).

### Example 5

### Alanine Scanning

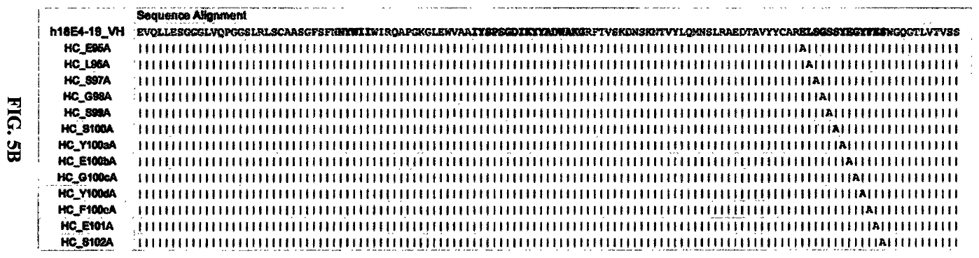

Alanine scanning variants of the humanized h18E4-19 antibody were generated to identify CDR3 residues important for binding to human CD38. Alanine mutants were systematically generated by mutating each residue of the heavy and light chain CDR3 regions to alanine as shown in FIGS. 5A-5B. Each mutant variant was tested for binding to CD38 by ELISA, as shown in Table 13. The variants with EC50 values of 0.02 nM-0.1 nM (class A) identify CDR3 positions that can be mutated without substantially inhibiting binding to CD38. The sequences of the alanine scanning variants are presented in Table 5 (HC CDRs), Table 6 (LC CDRs), Table 7 (VH), and Table (VL).

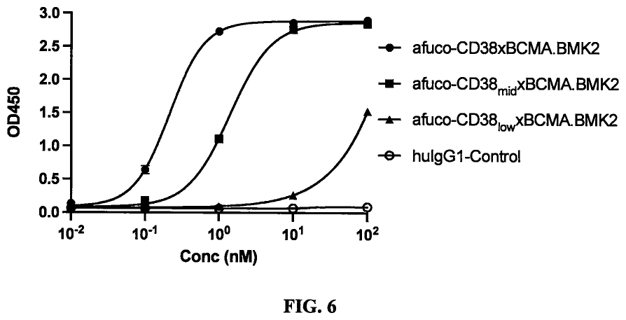

Binding of anti-CD38×BCMA bispecific antibody variants to human CD38, tested according to the methods described in Example 3, is illustrated in FIG. 6 relative to an isotype control human IgG1 antibody.

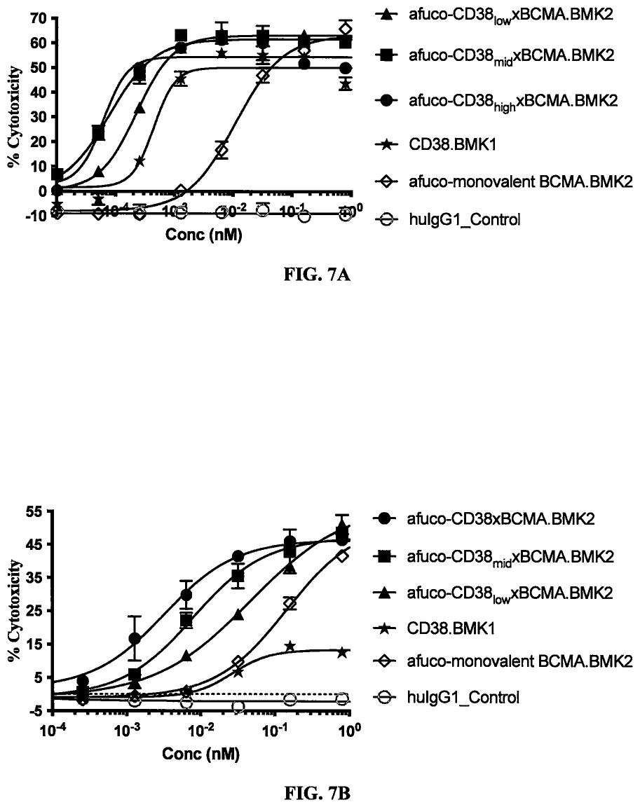

As characterized according to the methods described in Example 4, CD38×BCMA affinity variants exhibit higher ADCC mediated lysis of Daudi cells compared to a monovalent anti-BCMA antibodies, a bivalent monospecific anti-CD38 antibodies, and an isotype control human IgG1 antibody (FIG. 7A). CD38×BCMA affinity variants exhibit higher ADCC mediated lysis of U266B1 cells compared to a monovalent anti-BCMA antibodies, a bivalent monospecific anti-CD38 antibodies, and an isotype control human IgG1 antibody (FIG. 7B).

TABLE 13
Binding of humanized 18E4 alanine
variants to the human CD38 ECD,
as determined by ELISA. Amino acid
numbering is based on the Kabat
numbering system. EC50 ranges for
huCD38 - A: 0.02 nM-0.1 nM;
B: 0.1 nM-0.5 nM; C: >0.5 nM.

| Anti-CD38 Antibody | EC50 (nM) |
|---|---|
| hu18E4-19 | A |
| LC Q89A | A |
| LC G90A | B |
| LC Y91A | C |
| LC Y92A | A |
| LC S93A | B |
| LC G94A | C |
| LC G95A | A |
| LC S95aA | A |
| LC Y96A | B |
| HC E95A | C |
| HC L96A | A |
| HC S97A | A |
| HC G98A | A |
| HC S99A | A |
| HC S100A | A |
| HC Y100aA | A |
| HC E100bA | C |
| HC G100cA | B |
| HC Y100dA | C |
| HC F100eA | A |
| HC E101A | A |
| HC S102A | A |
| hIgG1 control | NB |

*NB: no binding, binding too low to quantify

### Example 6

### In Vivo Xenograft Studies

In vivo anti-tumor activities of the CD38/BCMA Bispecifics were investigated in a NCI-H929 Myeloma CDX model. The NCI-H929 cells were maintained in vitro as a monolayer culture in IMDM medium supplemented with 20% fetal bovine serum, 1% amino acids at 37° C. in an atmosphere of 5% CO₂ in air. The tumor cells were routinely subcultured twice weekly by trypsin-EDTA treatment. The cells growing in an exponential growth phase were harvested and counted for tumor inoculation. Each mouse was inoculated subcutaneously at the right flank with NCI-H929 cells (5×10⁶) in 0.2 mL of Matrigel (1:1) for tumor development. The animals were randomized and treatment was started when the average tumor volume reaches approximately 123 mm³ for the efficacy study.

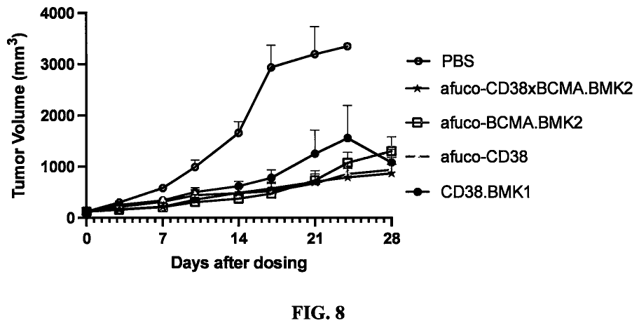

Animals in the study did not show obvious body weight loss. The therapeutic efficacy of test articles as a single agent in the treatment of the NCI-H929 human myeloma cancer xenograft model was evaluated. The results of tumor sizes in different groups at different time points after tumor inoculation are shown in the FIG. 8. The mean tumor size of the vehicle treated control mice reached 2939 mm³ at day 17 after the start of treatment. Treatment with CD38×BCMA.BMK2 (5 mg/kg), BCMA.BMK2 (5 mg/kg), anti-CD38 (5 mg/kg) and CD38.BMK1 (5 mg/kg) all produced significant antitumor activities; their mean tumor sizes were 573 mm³, 472 mm³, 521 mm³, and 780 mm³ respectively at the same time (T/C value=19.48%, 16.05%, 17.74% and 26.55%; TGI=84.03%, 87.58%, 85.84% and 76.63%) respectively; Thep values were all less than 0.001.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A multispecific antibody that comprises a CD38 binding domain and a BCMA binding domain.

2. The multispecific antibody of claim 1, wherein said multispecific antibody is bispecific, trispecific, or tetraspecific.

3. The multispecific antibody of claim 2, wherein said multispecific antibody is bispecific.

4. The multispecific antibody of claim 1, wherein said multispecific antibody is bivalent, trivalent, or tetravalent.

5. The multispecific antibody of claim 4, wherein said multispecific antibody is bivalent.

6. The multispecific antibody of claim 1, wherein said CD38 binding domain comprises an antibody, or functional fragment or functional variant thereof, that specifically binds CD38.

7. The multispecific antibody of claim 6, wherein said antibody, or functional fragment or functional variant thereof, comprises a variable domain of an IgG heavy chain and a variable domain of an IgG light chain.

8. The multispecific antibody of claim 7, wherein said variable domain of an IgG heavy chain comprises a variable domain of an IgG1, IgG2, IgG3, or IgG4 heavy chain; and said variable domain of an IgG light chain comprises a variable domain of an IgG1, IgG2, IgG3, or IgG4 light chain.

9. The multispecific antibody of claim 8, wherein said variable domain of an IgG heavy chain comprises a variable domain of an IgG1 heavy chain; and said variable domain of an IgG light chain comprises a variable domain of an IgG1 light chain.

10. The multispecific antibody of claim 6, wherein said antibody, or functional fragment or functional variant thereof, comprises a scFv or a Fab.

11. The multispecific antibody of claim 7, wherein said variable domain of an IgG heavy chain (HC) of said CD38 binding domain comprises complementarity determining regions (CDRs): HC-CDR1, HC-CDR2, and HC-CDR3, wherein the HC-CDR1, the HC-CDR2, and the HC-CDR3 of said heavy chain variable domain comprise a set of sequences selected from the group consisting of the following set of sequences:
a. HC-CDR1: SEQ ID NO: 9; HC-CDR2: SEQ ID NO: 10; HC-CDR3: SEQ ID NO: 11; and
b. HC-CDR1: SEQ ID NO: 12; HC-CDR2: SEQ ID NO: 13; HC-CDR3: SEQ ID NO: 14.

12. The multispecific antibody of claim 7, wherein said variable domain of an IgG light chain (LC) of said CD38 binding domain comprises complementarity determining regions (LC) (CDRs): LC-CDR1, LC-CDR2, and LC-CDR3, wherein the LC-CDR1, the LC-CDR2, and the LC-CDR3 of said heavy chain variable domain comprise a set of sequences selected from the group consisting of the following set of sequences:
a. LC-CDR1: SEQ ID NO: 67; LC-CDR2: SEQ ID NO: 68; LC-CDR3: SEQ ID NO: 69; and
b. LC-CDR1: SEQ ID NO: 70; LC-CDR2: SEQ ID NO: 71; LC-CDR3: SEQ ID NO: 72.

13. The multispecific antibody of claim 7, said variable domain of an IgG light chain (LC) of said CD38 binding domain comprises complementarity determining regions (CDRs): LC-CDR1, LC-CDR2, and LC-CDR3, and said variable domain of an IgG heavy chain (HC) of said CD38 binding domain comprises CDRs: HC-CDR1, HC-CDR2, and HC-CDR3, wherein said LC: LC-CDR1, LC-CDR2, and LC-CDR3 sequences and said HC: HC-CDR1, HC-CDR2, and HC-CDR3 sequences are as set forth in any one of the following sets of sequences:
a. HC-CDR1: SEQ ID NO: 9; HC-CDR2: SEQ ID NO: 10; HC-CDR3: SEQ ID NO: 11; LC-CDR1: SEQ ID NO: 67; LC-CDR2: SEQ ID NO: 68; and LC-CDR3: SEQ ID NO: 69; or
b. HC-CDR1: SEQ ID NO: 12; HC-CDR2: SEQ ID NO: 13; HC-CDR3: SEQ ID NO: 14; and LC-CDR1: SEQ ID NO: 70; LC-CDR2: SEQ ID NO: 71; and LC-CDR3: SEQ ID NO: 72.

14. The multispecific antibody of claim 7, wherein said variable domain of an IgG heavy chain of said CD38 binding domain comprises an amino acid sequence with at least 90%, 95%, 96%, 97%, 98%, 99%, or 100% identity to any one of SEQ ID NO: 114-136, 197-209.

15. The multispecific antibody of claim 7, wherein said variable domain of an IgG light chain of said CD38 binding domain comprises an amino acid sequence with at least 90%, 95%, 96%, 97%, 98%, 99%, or 100% identity to any one of SEQ ID NO: 137-162, 210-218.

16. The multispecific antibody of claim 7, wherein said CD38 binding domain comprises
a. a variable domain of an IgG heavy chain comprises an amino acid sequence with at least 90%, 95%, 96%, 97%, 98%, 99%, or 100% identity to SEQ ID NO: 114; and a variable domain of an IgG heavy chain comprises an amino acid sequence with at least 90%, 95%, 96%, 97%, 98%, 99%, or 100% identity to SEQ ID NO: 137; or
b. a variable domain of an IgG heavy chain comprises an amino acid sequence with at least 90%, 95%, 96%, 97%, 98%, 99%, or 100% identity to SEQ ID NO: 115; and a variable domain of an IgG heavy chain comprises an amino acid sequence with at least 90%, 95%, 96%, 97%, 98%, 99%, or 100% identity to SEQ ID NO: 138.

17. The multispecific antibody of claim 1, wherein said BCMA binding domain comprises an antibody, or functional fragment or functional variant thereof, that specifically binds BCMA.

18. The multispecific antibody of claim 17, wherein said antibody, or functional fragment or functional variant thereof, comprises a variable domain of an IgG heavy chain and a variable domain of an IgG light chain.

19. The multispecific antibody of claim 18, wherein said variable domain of an IgG heavy chain comprises a variable domain of an IgG1, IgG2, IgG3, or IgG4 heavy chain; and said variable domain of an IgG light chain comprises a variable domain of an IgG1, IgG2, IgG3, or IgG4 light chain.

20. The multispecific antibody of claim 19, wherein said variable domain of an IgG heavy chain comprises a variable domain of an IgG1 heavy chain; and said variable domain of an IgG light chain comprises a variable domain of an IgG1 light chain.

21. The multispecific antibody of claim 17, wherein said antibody, or functional fragment or functional variant thereof, comprises a scFv or a Fab.

22. The multispecific antibody of claim 18, wherein said variable domain of an IgG heavy chain (HC) of said BCMA binding domain comprises complementarity determining regions (CDRs): HC-CDR1, HC-CDR2, and HC-CDR3, wherein said HC-CDR1, the HC-CDR2, and the HC-CDR3 of said variable heavy chain comprise a set of sequences selected from the group consisting of the following set of sequences: HC-CDR1: SEQ ID NO: 1; HC-CDR2: SEQ ID NO: 2; HC-CDR3: SEQ ID NO: 3.

23. The multispecific antibody of claim 18, wherein said variable domain of an IgG light chain (LC) of said BCMA binding domain comprises complementarity determining regions (CDRs): LC-CDR1, LC-CDR2, and LC-CDR3, wherein the LC-CDR1, the LC-CDR2, and the LC-CDR3 of the heavy chain comprise a set of sequences selected from the group consisting of the following set of sequences: LC-CDR1: SEQ ID NO: 4; LC-CDR2: SEQ ID NO: 5; LC-CDR3: SEQ ID NO: 6.

24. The multispecific antibody of claim 18, said variable domain of an IgG light chain (LC) of said BCMA binding domain comprises complementarity determining regions (CDRs): LC-CDR1, LC-CDR2, and LC-CDR3, and said variable domain of an IgG heavy chain (HC) of said BCMA binding domain comprises complementarity determining regions HC-CDR1, HC-CDR2, and HC-CDR3, wherein said LC: LC-CDR1, LC-CDR2, and LC-CD3 sequences and said HC: HC-CDR1, HC-CDR2, and HC-CDR3 sequences comprise sequences with at least 90%, 95%, 96%, 97%, 98%, 99%, or 100% identity to sequences as set forth in any one of the following sets of sequences: HC-CDR1: SEQ ID NO: 1; HC-CDR2: SEQ ID NO: 2; HC-CDR3: SEQ ID NO: 3; LC-CDR1: SEQ ID NO: 4; LC-CDR2: SEQ ID NO: 5; and LC-CDR3: SEQ ID NO: 6.

25. The multispecific antibody of claim 18, wherein said variable domain of an IgG heavy chain of said BCMA binding domain comprises an amino acid sequence with at least 90%, 95%, 96%, 97%, 98%, 99%, or 100% identity to SEQ ID NO: 7.

26. The multispecific antibody of claim 18, wherein said variable domain of an IgG light chain of said BCMA binding domain comprises an amino acid sequence with at least 90%, 95%, 96%, 97%, 98%, 99%, or 100% identity to SEQ ID NO: 8.

27. The multispecific antibody of claim 18, wherein BCMA binding domain comprises a variable domain of an IgG heavy chain comprises an amino acid sequence with at least 90%, 95%, 96%, 97%, 98%, 99%, or 100% identity to SEQ ID NO: 7; and a variable domain of an IgG heavy chain comprises an amino acid sequence with at least 90%, 95%, 96%, 97%, 98%, 99%, or 100% identity to SEQ ID NO: 8.

28. The multispecific antibody of claim 1, further comprising an IgG hinge region, or a portion thereof.

29. The multispecific antibody of claim 1, further comprising an Fc region.

30. The multispecific antibody of claim 29, wherein said Fc region comprises an IgG CH2 domain and an IgG CH3 domain.

31. The multispecific antibody of claim 30, wherein said IgG hinge region is C terminal to said CD38 binding domain or said BCMA binding domain and N terminal to said Fc region.

32. The multispecific antibody of claim 29, wherein said Fc region comprises a heterodimeric Fc region.

33. The multispecific antibody of claim 29, wherein said Fc region comprises at least one amino acid modification that increases the half-life of the multispecific antibody.

34. The multispecific antibody of claim 29, wherein said Fc region comprises at least one amino acid modification that modulates its interaction with an Fc receptor.

35. The multispecific antibody of claim 29, wherein said Fc region comprises at least one amino acid modification that increases binding of said Fc region to an Fc receptor.

36. The multispecific antibody of claim 29, wherein said Fc region comprises at least one amino acid modification that decreases glycosylation of the Fe region.

37. The multispecific antibody of claim 36, wherein said modification is an amino acid substitution, deletion, or addition.

38. The multispecific antibody of claim 37, wherein said modification is an amino acid substitution.

39. The multispecific antibody of claim 38, wherein said at least one amino acid modification that decreases glycosylation of the Fc region comprises an amino acid substitution at a position corresponding to position N297 of human IgG1, wherein the numbering is according to the EU index of Kabat.

40. The multispecific antibody of claim 29, wherein said Fc region is afucosylated.

41. The multispecific antibody of claim 29, wherein said Fc region comprises at least one amino acid modification that increases antibody-dependent cellular cytotoxicity (ADCC).

42. The multispecific antibody of claim 41, wherein said modification is an amino acid substitution, deletion, or addition.

43. The multispecific antibody of claim 42, wherein said modification is an amino acid substitution.

44. The multispecific antibody of claim 43, wherein said Fc region comprises at least one mutation that increases antibody-dependent cellular cytotoxicity (ADCC), wherein said at least one mutation that increases ADCC comprises an amino acid substitution at positions corresponding to positions S239, I332, and A330 of human IgG1, wherein the amino acid numbering is according to the EU index according to Kabat et al.

45. The multispecific antibody of claim 44, wherein said amino acid substitutions are S239D, I332E, and A330L, wherein the amino acid numbering is according to the EU index according to Kabat et al.

46. The multispecific antibody of claim 29, comprising said heterodimeric Fc region, wherein said heterodimeric Fc region comprises a knob chain and a hole chain, forming a knob-in-hole (KIH) structure.

47. The multispecific antibody of claim 46, wherein said knob chain comprises an amino acid substitution at a position corresponding to T366 of IgG1, wherein amino acid position numbering is according to the EU index according to Kabat et al.

48. The multispecific antibody of claim 47, wherein said T366 substitution comprises a T336W mutation, wherein amino acid position numbering is according to the EU index according to Kabat et al.

49. The multispecific antibody of claim 46, wherein said hole chain comprises an amino acid substitution at a position corresponding to T366, L368, or Y407 of IgG1, wherein amino acid position numbering is according to the EU index according to Kabat et al.

50. The multispecific antibody of claim 49, wherein said hole chain comprises an amino acid substitution at a position corresponding to T366, L368, and Y407 of IgG1, wherein amino acid position numbering is according to the EU index according to Kabat et al.

51. The multispecific antibody of claim 49, wherein said T366, L368, or Y407 amino acid substitutions comprise a T366S, L368A, or Y407V of IgG1, wherein amino acid position numbering is according to the EU index according to Kabat et al.

52. The multispecific antibody of claim 50, wherein said T366, L368, and Y407 amino acid substitutions comprises a T366S, L368A, and Y407V of IgG1, wherein amino acid position numbering is according to the EU index according to Kabat et al.

53. The multispecific antibody of claim 1, wherein said multispecific antibody has a higher affinity for CD38 expressed on the surface of a cancer cell relative to a protein that comprises said CD38 binding domain but lacks an BCMA binding domain.

54. The multispecific antibody of claim 1, wherein said multispecific antibody binds to a target cell that expresses CD38 and BCMA with an enhanced affinity compared to that of a protein that comprises only the first component that specifically binds to CD38 or the second component that specifically binds to BCMA.

55. The multispecific antibody of claim 1, wherein said multispecific antibody exhibits a higher affinity for CD38 expressed on the surface of a target cancer cell than for CD38 expressed on the surface of a non-cancer cell.

56. The multispecific antibody of claim 55, wherein BCMA is expressed on the surface of said target cancer cell, wherein said binding is measured by flow cytometry.

57. The multispecific antibody of claim 1, wherein said multispecific antibody exhibits a higher affinity for CD38 expressed on the surface of a target plasma cell than for CD38 expressed on the surface of a non-plasma cell.

58. The multispecific antibody of claim 1, wherein said multispecific antibody exhibits a higher affmity for CD38 expressed on the surface of a target plasma cell than for CD38 expressed on the surface of a B cell that is not a plasma cell.

59. The multispecific antibody of claim 1, wherein said multispecific antibody exhibits a higher affinity for CD38 expressed on the surface of a target plasma cell that secretes antibodies that recognize an autoantigen than for CD38 expressed on the surface of a plasma cell that does not secrete antibodies that recognize an autoantigen.

60. The multispecific antibody of claim 57, wherein said target plasma cell is a plasmablast, differentiated plasma cell, or long lived plasma cell.

61. The multispecific antibody of claim 57, wherein said target plasma cell is a cancer cell.

62. The multispecific antibody of claim 57, wherein said target plasma cell secretes antibodies that recognize autoantigens.

63. The multispecific antibody of claim 57, wherein BCMA is expressed on the surface of said target plasma cell, wherein said binding is measured by flow cytometry.

64. The multispecific antibody of claim 1, wherein said multispecific antibody induces enhanced antibody-dependent cellular cytotoxicity (ADCC) activity on a target cell that expresses CD38 to BCMA compared to ADCC activity induced on said target cell by a monospecific antibody that comprises only one of the component that specifically binds to CD38 or the second component that specifically binds to BCMA.

65. The multispecific antibody of claim 64, wherein said multispecific antibody induces at least 5%, 10%, 15%, 20%, 25%, 30%, 25%, 40%, 45%, or 50% more ADCC activity than said control monospecific antibody.

66. The multispecific antibody of claim 1, wherein said multispecific antibody induces enhanced complement-dependent cytotoxicity (CDC) activity on a target cell that expresses CD38 to BCMA compared to CDC activity induced on said target cell by a protein that comprises only one of the component that specifically binds to CD38 or the second component that specifically binds to BCMA.

67. The multispecific antibody of claim 66, wherein said multispecific antibody induces at least 5%, 10%, 15%, 20%, 25%, 30%, 25%, 40%, 45%, or 50% more CDC activity than said control monospecific antibody.

68. The multispecific antibody of claim 64, wherein said target cell expresses CD38 and BCMA.

69. The multispecific antibody of claim 68, wherein said target cell expresses a lower level of CD38 relative to BCMA on the surface of said target cell.

70. The multispecific antibody of claim 68, wherein the ratio of BCMA to CD38 on the surface of said cancer cells is at least about 1, 1.5, 2.0, 2.5, 5, 10, 15, 20, 50, 100, or 200.

71. The multispecific antibody of claim 64, wherein said target cell is a cancer cell.

72. The multispecific antibody of claim 71, wherein said cancer is a hematological malignancy.

73. The multispecific antibody of claim 72, wherein said hematological malignancy is a B cell malignancy.

74. The multispecific antibody of claim 73, wherein said B cell malignancy is multiple myeloma.

75. The multispecific antibody of claim 64, wherein said target cell is a plasma cell.

76. The multispecific antibody of claim 75, wherein said plasma cell secretes antibodies that recognize autoantigens.

77. The multispecific antibody of claim 1, wherein said multispecific antibody binds to a cancer cell that expresses CD38 and BCMA on the surface, and wherein the ratio of BCMA to CD38 on the surface of said cancer cell is at least about 1, 1.5, 2.0, 2.5, 5, 10, 15, 20, 50, 100, or 200.

78. The multispecific antibody of claim 1, wherein said multispecific antibody binds to a plasma cell that expresses CD38 and BCMA on the surface, and wherein the ratio of BCMA to CD38 on the surface of said cancer cell is at least about 1, 1.5, 2.0, 2.5, 5, 10, 15, 20, 50, 100, or 200.

79. The multispecific antibody of claim 78, wherein said plasma cell is a plasmablast, differentiated plasma cell, or long lived plasma cell.

80. The multispecific antibody of claim 78, wherein said plasma cell is a cancer cell.

81. The multispecific antibody of claim 78, wherein said plasma cell secrete antibodies that recognize autoantigens.

82. A nucleic acid molecule encoding the multispecific antibody of claim 1.

83. A vector comprising the nucleic acid molecule of claim 82.

84. A pharmaceutical composition comprising the multispecific antibody of claim 1.

85. The pharmaceutical composition of claim 84, further comprising a pharmaceutically acceptable carrier, an excipient, or any combinations thereof.

86. A method of treating a subject having cancer, the method comprising: administering to the subject the multispecific antibody of claim 1.

87. The method of claim 86, wherein said cancer comprises cancer cells that express CD38 and BCMA.

88. The method of claim 87, wherein the ratio of BCMA to CD38 on the surface of said cancer cells is at least about 1, 1.5, 2.0, 2.5, 5, 10, 15, 20, 50, 100, or 200.

89. The method of claim 87, wherein said cancer cells that express CD38 and BCMA are lysed.

90. The method of claim 86, wherein the multispecific antibody induces antibody-dependent cellular cytotoxicity (ADCC) mediated killing of said cancer cells that express CD38 and BCMA.

91. The method of claim 86, wherein the multispecific antibody induces complement-dependent cytotoxicity (CDC) mediated killing of said cancer cells that express CD38 and BCMA.

92. The method of claim 86, wherein the cancer is a hematological malignancy.

93. The method of claim 92, wherein said cancer is B cell cancer.

94. The method of claim 93, wherein said cancer is multiple myeloma.

95. The method of claim 86, comprising administering to said subject an anti-cancer agent.

96. The method of claim 95, wherein said anti-cancer agent is a chemotherapeutic agent or a biologic agent.

97. A method of treating a subject having an autoimmune disease, said method comprising: administering to the subject the multispecific antibody of claim 1.

98. The method of claim 97, wherein said autoimmune disease is characterized by immune cells that express CD38 and BCMA.

99. The method of claim 98, wherein said immune cells are B cells.

100. The method of claim 99, wherein said B cells are plasma cells.

101. The method of claim 100, wherein said plasma cells comprise plasmablasts, differentiated plasma cells, or long lived plasma cells.

102. The method of claim 99, wherein said immune cells secrete antibodies that recognize autoantigens.

103. The method of claim 97, wherein the ratio of BCMA to CD38 on the surface of said immune cells is at least about 1, 1.5, 2.0, 2.5, 5, 10, 15, 20, 50, 100, or 200.

104. The method of claim 98, wherein said immune cells that express CD38 and BCMA are lysed.

105. The method of claim 97, wherein said multispecific antibody induces antibody-dependent cellular cytotoxicity (ADCC) mediated killing of said immune cells that express CD38 and BCMA.

106. The method of claim 97, wherein said multispecific antibody induces complement-dependent cytotoxicity (CDC) mediated killing of said immune cells that express CD38 and BCMA.

107. The method of claim 97, wherein said autoimmune disease is selected from the group consisting of rheumatoid arthritis (RA), systemic lupus erythematosus (SLE), multiple sclerosis (MS), pemphigus vulgaris (PV), myasthenia gravis (MG) and immune thrombocytopenic purpura (ITP).


SEQUENCE LISTING (224 sequences)

SEQ ID NO: 1 - protein, 5 aa, Artificial Sequence
  Description of Artificial Sequence Synthetic peptide
NYWMH

SEQ ID NO: 2 - protein, 17 aa, Artificial Sequence
  Description of Artificial Sequence Synthetic peptide
ATYRGHSDTYYNQKFKG

SEQ ID NO: 3 - protein, 12 aa, Artificial Sequence
  Description of Artificial Sequence Synthetic peptide
GAIYNGYDVLDN

SEQ ID NO: 4 - protein, 11 aa, Artificial Sequence
  Description of Artificial Sequence Synthetic peptide
SASQDISNYLN

SEQ ID NO: 5 - protein, 7 aa, Artificial Sequence
  Description of Artificial Sequence Synthetic peptide
YTSNLHS

SEQ ID NO: 6 - protein, 9 aa, Artificial Sequence
  Description of Artificial Sequence Synthetic peptide
QQYRKLPWT

SEQ ID NO: 7 - protein, 121 aa, Artificial Sequence
  Description of Artificial Sequence Synthetic polypeptide
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSS

SEQ ID NO: 8 - protein, 107 aa, Artificial Sequence
  Description of Artificial Sequence Synthetic polypeptide
DIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIK

SEQ ID NO: 9 - protein, 5 aa, Artificial Sequence
  Description of Artificial Sequence Synthetic peptide
NYWII

SEQ ID NO: 10 - protein, 18 aa, Artificial Sequence
  Description of Artificial Sequence Synthetic peptide
AIYSPSGDIKYYADWAKG

SEQ ID NO: 11 - protein, 13 aa, Artificial Sequence
  Description of Artificial Sequence Synthetic peptide
ELSGSSYEGYFES

SEQ ID NO: 12 - protein, 5 aa, Artificial Sequence
  Description of Artificial Sequence Synthetic peptide
SFAMS

SEQ ID NO: 13 - protein, 17 aa, Artificial Sequence
  Description of Artificial Sequence Synthetic peptide
AISGSGGGTYYADSVKG

SEQ ID NO: 14 - protein, 13 aa, Artificial Sequence
  Description of Artificial Sequence Synthetic peptide
DKILWFGEPVFDY

SEQ ID NO: 15 - protein, 5 aa, Artificial Sequence
  Description of Artificial Sequence Synthetic peptide
NYWIC

SEQ ID NO: 16 - protein, 18 aa, Artificial Sequence
  Description of Artificial Sequence Synthetic peptide
CIYSPSGDIKYYANWAKG

SEQ ID NO: 17 - protein, 5 aa, Artificial Sequence
  Description of Artificial Sequence Synthetic peptide
RYYVT

SEQ ID NO: 18 - protein, 16 aa, Artificial Sequence
  Description of Artificial Sequence Synthetic peptide
IIYISGTTYYATWAKG

SEQ ID NO: 19 - protein, 11 aa, Artificial Sequence
  Description of Artificial Sequence Synthetic peptide
AWPVGTYVLPL

SEQ ID NO: 20 - protein, 5 aa, Artificial Sequence
  Description of Artificial Sequence Synthetic peptide
SYYMS

SEQ ID NO: 21 - protein, 16 aa, Artificial Sequence
  Description of Artificial Sequence Synthetic peptide
FISKTAITYYASWARG

SEQ ID NO: 22 - protein, 9 aa, Artificial Sequence
  Description of Artificial Sequence Synthetic peptide
VDAYSAGDL

SEQ ID NO: 23 - protein, 5 aa, Artificial Sequence
  Description of Artificial Sequence Synthetic peptide
SYAMG

SEQ ID NO: 24 - protein, 16 aa, Artificial Sequence
  Description of Artificial Sequence Synthetic peptide
IMTSGGNIYYANWAKG

SEQ ID NO: 25 - protein, 16 aa, Artificial Sequence
  Description of Artificial Sequence Synthetic peptide
EREFYGGGTSGSRLDL

SEQ ID NO: 26 - protein, 6 aa, Artificial Sequence
  Description of Artificial Sequence Synthetic peptide
SRYWIC

SEQ ID NO: 27 - protein, 17 aa, Artificial Sequence
  Description of Artificial Sequence Synthetic peptide
CIVAGTTNTYYASWAKG

SEQ ID NO: 28 - protein, 13 aa, Artificial Sequence
  Description of Artificial Sequence Synthetic peptide
DPRTGSNVGYFNL

SEQ ID NO: 29 - protein, 5 aa, Artificial Sequence
  Description of Artificial Sequence Synthetic peptide
NYGVS

SEQ ID NO: 30 - protein, 16 aa, Artificial Sequence
  Description of Artificial Sequence Synthetic peptide
YILTSGGTYYANWAQG

SEQ ID NO: 31 - protein, 12 aa, Artificial Sequence
  Description of Artificial Sequence Synthetic peptide
PKDSDSSAFVSL

SEQ ID NO: 32 - protein, 7 aa, Artificial Sequence
  Description of Artificial Sequence Synthetic peptide
TYYYYMC

SEQ ID NO: 33 - protein, 17 aa, Artificial Sequence
  Description of Artificial Sequence Synthetic peptide
CTYTGDGATYYATWAKG

SEQ ID NO: 34 - protein, 13 aa, Artificial Sequence
  Description of Artificial Sequence Synthetic peptide
SADNSIYYGYFNL

SEQ ID NO: 35 - protein, 5 aa, Artificial Sequence
  Description of Artificial Sequence Synthetic peptide
NNAIS

SEQ ID NO: 36 - protein, 16 aa, Artificial Sequence
  Description of Artificial Sequence Synthetic peptide
SIYGSGNTYYATWAKG

SEQ ID NO: 37 - protein, 11 aa, Artificial Sequence
  Description of Artificial Sequence Synthetic peptide
EGAGSSWGFNL

SEQ ID NO: 38 - protein, 5 aa, Artificial Sequence
  Description of Artificial Sequence Synthetic peptide
NNAIS

SEQ ID NO: 39 - protein, 16 aa, Artificial Sequence
  Description of Artificial Sequence Synthetic peptide
SIYGTGNTYYATWAKG

SEQ ID NO: 40 - protein, 11 aa, Artificial Sequence
  Description of Artificial Sequence Synthetic peptide
EGAGSIWGFNL

SEQ ID NO: 41 - protein, 5 aa, Artificial Sequence
  Description of Artificial Sequence Synthetic peptide
NYDMT

SEQ ID NO: 42 - protein, 16 aa, Artificial Sequence
  Description of Artificial Sequence Synthetic peptide
VISSGDNTNYARWAKG

SEQ ID NO: 43 - protein, 12 aa, Artificial Sequence
  Description of Artificial Sequence Synthetic peptide
ILYNKGRYYFTF

SEQ ID NO: 44 - protein, 6 aa, Artificial Sequence
  Description of Artificial Sequence Synthetic peptide
NIYVMC

SEQ ID NO: 45 - protein, 17 aa, Artificial Sequence
  Description of Artificial Sequence Synthetic peptide
CIGTGSGDTDYATWAKG

SEQ ID NO: 46 - protein, 9 aa, Artificial Sequence
  Description of Artificial Sequence Synthetic peptide
DPGAGTWNL

SEQ ID NO: 47 - protein, 6 aa, Artificial Sequence
  Description of Artificial Sequence Synthetic peptide
SAYDMC

SEQ ID NO: 48 - protein, 18 aa, Artificial Sequence
  Description of Artificial Sequence Synthetic peptide
CLYTVSSDSIYYASWAKG

SEQ ID NO: 49 - protein, 7 aa, Artificial Sequence
  Description of Artificial Sequence Synthetic peptide
DGDYFAL

SEQ ID NO: 50 - protein, 5 aa, Artificial Sequence
  Description of Artificial Sequence Synthetic peptide
IYTMA

SEQ ID NO: 51 - protein, 16 aa, Artificial Sequence
  Description of Artificial Sequence Synthetic peptide
IISGYGTTYYATWAKG

SEQ ID NO: 52 - protein, 8 aa, Artificial Sequence
  Description of Artificial Sequence Synthetic peptide
TTVQSTDL

SEQ ID NO: 53 - protein, 5 aa, Artificial Sequence
  Description of Artificial Sequence Synthetic peptide
SYDMS

SEQ ID NO: 54 - protein, 16 aa, Artificial Sequence
  Description of Artificial Sequence Synthetic peptide
YITYGGNIYYATWAKG

SEQ ID NO: 55 - protein, 12 aa, Artificial Sequence
  Description of Artificial Sequence Synthetic peptide
TLYTGGRYYFSL

SEQ ID NO: 56 - protein, 6 aa, Artificial Sequence
  Description of Artificial Sequence Synthetic peptide
SNDAIC

SEQ ID NO: 57 - protein, 17 aa, Artificial Sequence
  Description of Artificial Sequence Synthetic peptide
CIYAGSGNTYYASWAKG

SEQ ID NO: 58 - protein, 9 aa, Artificial Sequence
  Description of Artificial Sequence Synthetic peptide
ADTIDYFNL

SEQ ID NO: 59 - protein, 6 aa, Artificial Sequence
  Description of Artificial Sequence Synthetic peptide
SGCDMC

SEQ ID NO: 60 - protein, 17 aa, Artificial Sequence
  Description of Artificial Sequence Synthetic peptide
CIYTGSGSTYYANWAKG

SEQ ID NO: 61 - protein, 7 aa, Artificial Sequence
  Description of Artificial Sequence Synthetic peptide
DSDYLGL

SEQ ID NO: 62 - protein, 18 aa, Artificial Sequence
  Description of Artificial Sequence Synthetic peptide
CIYSPSGDIKYYADSVKG

SEQ ID NO: 63 - protein, 5 aa, Artificial Sequence
  Description of Artificial Sequence Synthetic peptide
NYWIA

SEQ ID NO: 64 - protein, 18 aa, Artificial Sequence
  Description of Artificial Sequence Synthetic peptide
SIYSPSGDIKYYADWAKG

SEQ ID NO: 65 - protein, 5 aa, Artificial Sequence
  Description of Artificial Sequence Synthetic peptide
NYWIS

SEQ ID NO: 66 - protein, 18 aa, Artificial Sequence
  Description of Artificial Sequence Synthetic peptide
YIYSPSGDIKYYADWAKG

SEQ ID NO: 67 - protein, 13 aa, Artificial Sequence
  Description of Artificial Sequence Synthetic peptide
RSSQSVYGHNWLA

SEQ ID NO: 68 - protein, 7 aa, Artificial Sequence
  Description of Artificial Sequence Synthetic peptide
RASNLAS

SEQ ID NO: 69 - protein, 10 aa, Artificial Sequence
  Description of Artificial Sequence Synthetic peptide
QGYYSGGSYA

SEQ ID NO: 70 - protein, 11 aa, Artificial Sequence
  Description of Artificial Sequence Synthetic peptide
RASQSVSSYLA

SEQ ID NO: 71 - protein, 7 aa, Artificial Sequence
  Description of Artificial Sequence Synthetic peptide
DASNRAT

SEQ ID NO: 72 - protein, 8 aa, Artificial Sequence
  Description of Artificial Sequence Synthetic peptide
QRSNWPPT

SEQ ID NO: 73 - protein, 13 aa, Artificial Sequence
  Description of Artificial Sequence Synthetic peptide
QCSQSVYGHNWLA

SEQ ID NO: 74 - protein, 10 aa, Artificial Sequence
  Description of Artificial Sequence Synthetic peptide
QGYYNGGSYA

SEQ ID NO: 75 - protein, 13 aa, Artificial Sequence
  Description of Artificial Sequence Synthetic peptide
QSSQSVVNANNLS

SEQ ID NO: 76 - protein, 7 aa, Artificial Sequence
  Description of Artificial Sequence Synthetic peptide
LASTLAS

SEQ ID NO: 77 - protein, 11 aa, Artificial Sequence
  Description of Artificial Sequence Synthetic peptide
LGVYDDDGDNA

SEQ ID NO: 78 - protein, 13 aa, Artificial Sequence
  Description of Artificial Sequence Synthetic peptide
QASQSVYSDNRLS

SEQ ID NO: 79 - protein, 7 aa, Artificial Sequence
  Description of Artificial Sequence Synthetic peptide
STSSLAS

SEQ ID NO: 80 - protein, 13 aa, Artificial Sequence
  Description of Artificial Sequence Synthetic peptide
QGEFICTSADCFV

SEQ ID NO: 81 - protein, 11 aa, Artificial Sequence
  Description of Artificial Sequence Synthetic peptide
QASQSIYNFLN

SEQ ID NO: 82 - protein, 7 aa, Artificial Sequence
  Description of Artificial Sequence Synthetic peptide
YASTLAF

SEQ ID NO: 83 - protein, 12 aa, Artificial Sequence
  Description of Artificial Sequence Synthetic peptide
QQGWNSGILDNS

SEQ ID NO: 84 - protein, 13 aa, Artificial Sequence
  Description of Artificial Sequence Synthetic peptide
QASQSVYNNNYLA

SEQ ID NO: 85 - protein, 7 aa, Artificial Sequence
  Description of Artificial Sequence Synthetic peptide
SASTLAS

SEQ ID NO: 86 - protein, 10 aa, Artificial Sequence
  Description of Artificial Sequence Synthetic peptide
QAYYSGGIYA

SEQ ID NO: 87 - protein, 11 aa, Artificial Sequence
  Description of Artificial Sequence Synthetic peptide
QASESIYSNLA

SEQ ID NO: 88 - protein, 7 aa, Artificial Sequence
  Description of Artificial Sequence Synthetic peptide
KASTLAS

SEQ ID NO: 89 - protein, 12 aa, Artificial Sequence
  Description of Artificial Sequence Synthetic peptide
QANHMIVIYGNG

SEQ ID NO: 90 - protein, 11 aa, Artificial Sequence
  Description of Artificial Sequence Synthetic peptide
QASENIYSSLA

SEQ ID NO: 91 - protein, 7 aa, Artificial Sequence
  Description of Artificial Sequence Synthetic peptide
RASTLAS

SEQ ID NO: 92 - protein, 12 aa, Artificial Sequence
  Description of Artificial Sequence Synthetic peptide
QTYYGSTSTGFT

SEQ ID NO: 93 - protein, 13 aa, Artificial Sequence
  Description of Artificial Sequence Synthetic peptide
QSSESVYKNNYLS

SEQ ID NO: 94 - protein, 10 aa, Artificial Sequence
  Description of Artificial Sequence Synthetic peptide
AGGYSGNING

SEQ ID NO: 95 - protein, 10 aa, Artificial Sequence
  Description of Artificial Sequence Synthetic peptide
AGGYTGNING

SEQ ID NO: 96 - protein, 13 aa, Artificial Sequence
  Description of Artificial Sequence Synthetic peptide
QSSQSIANSDELA

SEQ ID NO: 97 - protein, 7 aa, Artificial Sequence
  Description of Artificial Sequence Synthetic peptide
DASTLAP

SEQ ID NO: 98 - protein, 12 aa, Artificial Sequence
  Description of Artificial Sequence Synthetic peptide
QGTVYDSGWYAA

SEQ ID NO: 99 - protein, 11 aa, Artificial Sequence
  Description of Artificial Sequence Synthetic peptide
QASQTIGSRLA

SEQ ID NO: 100 - protein, 13 aa, Artificial Sequence
  Description of Artificial Sequence Synthetic peptide
QSYYYTSTSYPNA

SEQ ID NO: 101 - protein, 11 aa, Artificial Sequence
  Description of Artificial Sequence Synthetic peptide
QASQNIGGYLS

SEQ ID NO: 102 - protein, 13 aa, Artificial Sequence
  Description of Artificial Sequence Synthetic peptide
QTYYYSGSSRYWA

SEQ ID NO: 103 - protein, 11 aa, Artificial Sequence
  Description of Artificial Sequence Synthetic peptide
QASQNIYSNLA

SEQ ID NO: 104 - protein, 14 aa, Artificial Sequence
  Description of Artificial Sequence Synthetic peptide
QSYYGATSSSFGYG

SEQ ID NO: 105 - protein, 13 aa, Artificial Sequence
  Description of Artificial Sequence Synthetic peptide
QSSQSIANSNEVA

SEQ ID NO: 106 - protein, 7 aa, Artificial Sequence
  Description of Artificial Sequence Synthetic peptide
DASTLAS

SEQ ID NO: 107 - protein, 12 aa, Artificial Sequence
  Description of Artificial Sequence Synthetic peptide
QGTVYDNVWYAA

SEQ ID NO: 108 - protein, 10 aa, Artificial Sequence
  Description of Artificial Sequence Synthetic peptide
QAYYRDPTTA

SEQ ID NO: 109 - protein, 11 aa, Artificial Sequence
  Description of Artificial Sequence Synthetic peptide
QASQNIGNYLA

SEQ ID NO: 110 - protein, 15 aa, Artificial Sequence
  Description of Artificial Sequence Synthetic peptide
QSYYYTTDDNYRSWA

SEQ ID NO: 111 - protein, 13 aa, Artificial Sequence
  Description of Artificial Sequence Synthetic peptide
RCSQSVYGHNWLA

SEQ ID NO: 112 - protein, 10 aa, Artificial Sequence
  Description of Artificial Sequence Synthetic peptide
QGYYQGGSYA

SEQ ID NO: 113 - protein, 10 aa, Artificial Sequence
  Description of Artificial Sequence Synthetic peptide
QGYYNAGSYA

SEQ ID NO: 114 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVSS

SEQ ID NO: 115 - protein, 122 aa, Artificial Sequence [sequence omitted]

SEQ ID NO: 116 - protein, 121 aa, Artificial Sequence
  Description of Artificial Sequence Synthetic polypeptide
QSLEESGGDLVKPGASLTLTCTASGFSFNNYWICWIRQAPGKGLEWVACIYSPSGDIKYYANWAKGRFTVSKTSSTTVTLQMTSLTGADTATYFCARELSGSSYEGYFESWGPGTLVTVSS

SEQ ID NO: 117 - protein, 123 aa, Artificial Sequence
  Description of Artificial Sequence Synthetic polypeptide
EVQLLESGGGLVQPGGSLRLSCAASGFSFNNYWICWVRQAPGKGLEWVSCIYSPSGDIKYYADSVKGRFTISRDNSKNTLYLQMNSLRAEDTAVYYCAKELSGSSYEGYFESWGQGTLVTVSS

SEQ ID NO: 118 - protein, 123 aa, Artificial Sequence
  Description of Artificial Sequence Synthetic polypeptide
EVQLLESGGGLVQPGGSLRLSCAASGFSFNNYWICWIRQAPGKGLEWVACIYSPSGDIKYYADSVKGRFTVSKDNSKNTVYLQMNSLRAEDTAVYYCARELSGSSYEGYFESWGQGTLVTVSS

SEQ ID NO: 119 - protein, 123 aa, Artificial Sequence
  Description of Artificial Sequence Synthetic polypeptide
EVQLLESGGGLVQPGGSLRLSCAASGFSFNNYWICWIRQAPGKGLEWVACIYSPSGDIKYYADWAKGRFTVSKDNSKNTVYLQMNSLRAEDTAVYYCARELSGSSYEGYFESWGQGTLVTVSS

SEQ ID NO: 120 - protein, 123 aa, Artificial Sequence
  Description of Artificial Sequence Synthetic polypeptide
EVQLLESGGGLVQPGGSLRLSCAASGFSFNNYWIAWIRQAPGKGLEWVASIYSPSGDIKYYADWAKGRFTVSKDNSKNTVYLQMNSLRAEDTAVYYCARELSGSSYEGYFESWGQGTLVTVSS

SEQ ID NO: 121 - protein, 123 aa, Artificial Sequence
  Description of Artificial Sequence Synthetic polypeptide
EVQLLESGGGLVQPGGSLRLSCAASGFSFNNYWISWIRQAPGKGLEWVAYIYSPSGDIKYYADWAKGRFTVSKDNSKNTVYLQMNSLRAEDTAVYYCARELSGSSYEGYFESWGQGTLVTVSS

SEQ ID NO: 122 - protein, 116 aa, Artificial Sequence
  Description of Artificial Sequence Synthetic polypeptide
QSVEESGGRLVTPGTPLTLTCTASGFSLSRYYVTWVRQAPGKGLEWIGIIYISGTTYYATWAKGRFTISKSATTVDLRIASPTTEDTATYFCAAAWPVGTYVLPLWGPGTLVTVSS

SEQ ID NO: 123 - protein, 114 aa, Artificial Sequence
  Description of Artificial Sequence Synthetic polypeptide
QSVEESGGRLVTPGTPLTLTCTASGFSLSSYYMSWVRQAPGKGLEWIGFISKTAITYYASWARGRFTISKTSTAVDLKITSPTTEDTATYFCARVDAYSAGDLWGPGTLVTVSS

SEQ ID NO: 124 - protein, 121 aa, Artificial Sequence
  Description of Artificial Sequence Synthetic polypeptide
QSVEESGGRLVTPGTPLTLTCTVSGIDLNSYAMGWVRQAPGKGLKYIGIMTSGGNIYYANWAKGRFTISKTSTTVDLRITSPTTEDTATYFCAREREFYGGGTSGSRLDLWGQGTLVTVSS

SEQ ID NO: 125 - protein, 121 aa, Artificial Sequence
  Description of Artificial Sequence Synthetic polypeptide
QSLEESGGDLVKPGASLTLTCTASGFSFSSRYWICWVRQAPGKGLEWIACIVAGTTNTYYASWAKGRFTISKTSSTTVTLQMTSLTAADTATYFCAGDPRTGSNVGYFNLWGPGTLVTVSS

SEQ ID NO: 126 - protein, 117 aa, Artificial Sequence
  Description of Artificial Sequence Synthetic polypeptide
QSVEESGGRLVTPGTPLTLTCTVSGFSLSNYGVSWVRQAPGKGLEWIGYILTSGGTYYANWAQGRFTISKTSTTVDLKITSPTTEDTATYFCGRPKDSDSSAFVSLWGPGTLVTVSS

SEQ ID NO: 127 - protein, 122 aa, Artificial Sequence
  Description of Artificial Sequence Synthetic polypeptide
QSLEESGGDLVKPGASLTLTCTASGFSATTYYYYMCWVRQAPGKGLEWIACTYTGDGATYYATWAKGRFTISKTSSTTVTLQMTSLTAADTATYFCARSADNSIYYGYFNLWGPGTLVTVSS

SEQ ID NO: 128 - protein, 116 aa, Artificial Sequence
  Description of Artificial Sequence Synthetic polypeptide
QSVEESGGRLVTPGTPLTLTCTVSGFSLSNNAISWVRQAPGKGLQWIGSIYGSGNTYYATWAKGRFTVSKTSTTVDLKINSPTTEDTATYFCAREGAGSSWGFNLWGPGTLVTVSS

SEQ ID NO: 129 - protein, 116 aa, Artificial Sequence
  Description of Artificial Sequence Synthetic polypeptide
QSVEESGGRLVTPGTPLTLTCTVSGFSLNNNAISWVRQAPGKGLQWIGSIYGTGNTYYATWAKGRFSVSKTSTTVDLKINSPTTEDTATYFCATEGAGSIWGFNLWGPGTLVTVSS

SEQ ID NO: 130 - protein, 118 aa, Artificial Sequence
  Description of Artificial Sequence Synthetic polypeptide
QSVEESGGRLVTPGTPLTLTCTVSGFSLSNYDMTWVRQAPGRGLEWIGVISSGDNTNYARWAKGRFTISKTSSTTVDLKITSPTTEDTATYFCARILYNKGRYYFTFWGPGTLVTVSS

SEQ ID NO: 131 - protein, 117 aa, Artificial Sequence
  Description of Artificial Sequence Synthetic polypeptide
QELVESGGGLFQPGGSLALTCKASGFSLSNIYVMCWVRQAPGKGLEWIACIGTGSGDTDYATWAKGRFTISKTSSTTVTLQMTSLTAADTATYFCARDPGAGTWNLWGPGTLVTVSS

SEQ ID NO: 132 - protein, 117 aa, Artificial Sequence
  Description of Artificial Sequence Synthetic polypeptide
QEQLEESGGDLVKPGASLTLTCKASGFSFSSAYDMCWVRQAPGKGLEWVACLYTVSSDSIYYASWAKGRFTISRTSSTTVTLQMTSLTAADTATYFCARDGDYFALWGPGTLVTVSS

SEQ ID NO: 133 - protein, 113 aa, Artificial Sequence
  Description of Artificial Sequence Synthetic polypeptide
QSLEESGGRLVTPGTPLTLTCTVSGIDLSIYTMAWVRQAPGKGLEYIGIISGYGTTYYATWAKGRFIVSKTSTTVDLKITSPTTEDTATYFCVRTTVQSTDLWGPGTLVTVSS

SEQ ID NO: 134 - protein, 117 aa, Artificial Sequence
  Description of Artificial Sequence Synthetic polypeptide
RSLEESGGRLVTPGTPLTLTCTTSGFSLSSYDMSWVRQAPGKGLEWIGYITYGGNIYYATWAKGRFTTSKTSTTVDLKITSPTTEDTATYFCARTLYTGGRYYFSLWGPGTLVTVSS

SEQ ID NO: 135 - protein, 117 aa, Artificial Sequence
  Description of Artificial Sequence Synthetic polypeptide
QEQLEESGGDLVKPEGSLTLTCTASGFSFSNDAICWVRQAPGKGLEWIACIYAGSGNTYYASWAKGRFSISKTSSTTVTLQMTSLTVADTATYFCASADTIDYFNLWGPGTLVTVSS

SEQ ID NO: 136 - protein, 115 aa, Artificial Sequence
  Description of Artificial Sequence Synthetic polypeptide
QSLEESGGGLVKPGASLTLTCKASGFSFSSGCDMCWVRQAPGKGLEWISCIYTGSGSTYYANWAKGRFTISRTSSTTVTLQMTSLTAADTATYFCAGDSDYLGLWGPGTLVTVSS

SEQ ID NO: 137 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSYAFGGGTKVEIK

SEQ ID NO: 138 - protein, 107 aa, Artificial Sequence [sequence omitted]

SEQ ID NO: 139 - protein, 110 aa, Artificial Sequence
  Description of Artificial Sequence Synthetic polypeptide
AQVLTQTPSSVSAAVGGTVTINCQCSQSVYGHNWLAWYQHKPGQPPKLLMYRASNLASGVPSRFKGSGSGSQFTLTIGEVQSDDAATYYCQGYYNGGSYAFGGGTEVVVR

SEQ ID NO: 140 - protein, 110 aa, Artificial Sequence
  Description of Artificial Sequence Synthetic polypeptide
DIQMTQSPSTLSASVGDRVTITCRCSQSVYGHNWLAWYQQKPGKAPKLLIYRASNLASGVPSRFSGSGSGTEFTLTISSLQPDDFATYYCQGYYNGGSYAFGGGTKVEIK

SEQ ID NO: 141 - protein, 110 aa, Artificial Sequence
  Description of Artificial Sequence Synthetic polypeptide
DIQLTQSPSTLSASVGDRVTITCRCSQSVYGHNWLAWYQQKPGKAPKLLIYRASNLASGVPSRFSGSGSGTEFTLTISSVQPDDFATYYCQGYYNGGSYAFGGGTKVEIK

SEQ ID NO: 142 - protein, 110 aa, Artificial Sequence
  Description of Artificial Sequence Synthetic polypeptide
DIQLTQSPSTLSASVGDRVTITCRCSQSVYGHNWLAWYQQKPGKAPKLLMYRASNLASGVPSRFSGSGSGTEFTLTISSVQPDDFATYYCQGYYNGGSYAFGGGTKVEIK

SEQ ID NO: 143 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLIYRASNLASGVPSRFSGSGSGTEFTLTISSVQPDDFATYYCQGYYNGGSYAFGGGTKVEIK

SEQ ID NO: 144 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYNGGSYAFGGGTKVEIK

SEQ ID NO: 145 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYQGGSYAFGGGTKVEIK

SEQ ID NO: 146 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDAATYYCQGYYNGGSYAFGGGTKVEIK

SEQ ID NO: 147 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYNAGSYAFGGGTKVEIK

SEQ ID NO: 148 - protein, 111 aa, Artificial Sequence
  Description of Artificial Sequence Synthetic polypeptide
AAVLTQTPSPVSAAVGGTVTIKCQSSQSVVNANNLSWYQQKPGQPPKLLIYLASTLASGVPSRFSGSGSGTQFTLTISSVQSDDAATYYCLGVYDDDGDNAFGGGTEVVVK

SEQ ID NO: 149 - protein, 113 aa, Artificial Sequence
  Description of Artificial Sequence Synthetic polypeptide
AQVLTQTPSSVSAAVGGTVTINCQASQSVYSDNRLSWFQQKSGQPPKLLIYSTSSLASGVPSRFSGSGSGTQFTLTISGVQSDDAASYYCQGEFICTSADCFVFGGGTEVVVK

SEQ ID NO: 150 - protein, 110 aa, Artificial Sequence
  Description of Artificial Sequence Synthetic polypeptide
AYDMTQTPASVEAAVGGTVTIKCQASQSIYNFLNWYQQKPGQPPKLLIYYASTLAFGVPSRFKGSGSGTEYTLTISGVESADAATYYCQQGWNSGILDNSFGGGTEVVVK

SEQ ID NO: 151 - protein, 110 aa, Artificial Sequence
  Description of Artificial Sequence Synthetic polypeptide
AQVLTQTPSPVSAAVGGTVTINCQASQSVYNNNYLAWFQQKPGQPPKLLIYSASTLASGVPSRFKGSGSGTQFTLTISEVQSDDAATYYCQAYYSGGIYAFGGGTEVVVK

SEQ ID NO: 152 - protein, 111 aa, Artificial Sequence
  Description of Artificial Sequence Synthetic polypeptide
ADVVMTQTPASVSEPVGGTVTINCQASESIYSNLAWYQQKPGQPPKLLIYKASTLASGVSSRFKGSGSGTEFTLTISDLESADAATYYCQANHMIVIYGNGFGGGTEVVVK

SEQ ID NO: 153 - protein, 110 aa, Artificial Sequence
  Description of Artificial Sequence Synthetic polypeptide
AFELTQTPFSVSEPVGGTVTINCQASENIYSSLAWYQQKPGQPPKLLIYRASTLASGVPSRFSGSGSGTEFTLTISGVQSDDAATYYCQTYYGSTSTGFTFGGGTEVVVK

SEQ ID NO: 154 - protein, 110 aa, Artificial Sequence
  Description of Artificial Sequence Synthetic polypeptide
AQVLTQTASSVSAAVGGTVTISCQSSESVYKNNYLSWYQQKPGQPLKCLIYSASTLASGVPSRFKGSGSGTQFTLTISDLESDDAATYYCAGGYSGNINGFGGGTEVVVK

SEQ ID NO: 155 - protein, 110 aa, Artificial Sequence
  Description of Artificial Sequence Synthetic polypeptide
AQVLTQTASSVSAAVGGTVTISCQSSESVYKNNYLSWYQQKPGQPPKGLIYSASTLASGVPSRFKGSGSGTQFTLTISDLESDDAATYYCAGGYTGNINGFGGGTEVVVK

SEQ ID NO: 156 - protein, 112 aa, Artificial Sequence
  Description of Artificial Sequence Synthetic polypeptide
AQVLTQTPSSVSAAVGGTVTINCQSSQSIANSDELAWYQQKPGQPPKLLIYDASTLAPGVPSRFSGSGSGTQFTLTISGVQSDDAATYYCQGTVYDSGWYAAFGGGTEVVVK

SEQ ID NO: 157 - protein, 112 aa, Artificial Sequence
  Description of Artificial Sequence Synthetic polypeptide
ADIVMTQTPSSVEAAVGGTVTIKCQASQTIGSRLAWYQQKPGQPPKLLIYSASTLASGVSSRFKGSGSGTQFTLTISDLDSADAATYYCQSYYYTSTSYPNAFGGGTEVVVK

SEQ ID NO: 158 - protein, 112 aa, Artificial Sequence
  Description of Artificial Sequence Synthetic polypeptide
ADIVMTHTPASVEAAVGGTVTIKCQASQNIGGYLSWYQQKPGQRPKLLIYRASTLASGVPSRFKGSGSGTQFTLTISDLESADAATYYCQTYYYSGSSRYWAFGGGTEVVVK

SEQ ID NO: 159 - protein, 113 aa, Artificial Sequence
  Description of Artificial Sequence Synthetic polypeptide
ADIVMTQTPASVEAAVGGTVTINCQASQNIYSNLAWYQQKPGQRPKLLIYKASTLASGVSSRFKGSGSGTEFTLTISDLASADAATYYCQSYYGATSSSFGYGFGGGTEVVVK

SEQ ID NO: 160 - protein, 112 aa, Artificial Sequence
  Description of Artificial Sequence Synthetic polypeptide
AQVLTQTPSSVSAVVGGTVTINCQSSQSIANSNEVAWYQQKLGQPPKLLIYDASTLASGVPSRFSGSGSGTQFTLIISGVQSDDAATYYCQGTVYDNVWYAAFGGGTEVVVK

SEQ ID NO: 161 - protein, 110 aa, Artificial Sequence
  Description of Artificial Sequence Synthetic polypeptide
AQVLTQTPSPVSAAVGGTVTINCQASQSVYNNNYLAWYQQKPGQPPKLLIYRASNLASGVPSRFSASGSGTQFTLTISEVQSDDAATYYCQAYYRDPTTAFGGGTEVVVK

SEQ ID NO: 162 - protein, 114 aa, Artificial Sequence
  Description of Artificial Sequence Synthetic polypeptide
ADIVMTQTPSSVEAAVGGTVTIKCQASQNIGNYLAWYQQKPGQPPKVLIYRASTLASGVPSRFKGSGSGTHFTLTISDLESADAATYYCQSYYYTTDDNYRSWAFGGGTEVVVK

SEQ ID NO: 163 - protein, 452 aa, Artificial Sequence
  Description of Artificial Sequence Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 490 aa, Artificial Sequence
  Description of Artificial Sequence Synthetic polypeptide
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSSASTKGPRGSTSGGGSGGGSGGGGSSDIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIKRGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 166 - protein, 490 aa, Artificial Sequence
  Description of Artificial Sequence Synthetic polypeptide
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSSASTKGPRGSTSGGGSGGGSGGGGSSDIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIKRGGSGGSGGASKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 167 - protein, 451 aa, Artificial Sequence
  Description of Artificial Sequence Synthetic polypeptide
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 214 aa, Artificial Sequence
  Description of Artificial Sequence Synthetic polypeptide
DIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 484 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSYAFGGGTKVEIKGGGGSGGGGSGGGGSEVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 170 - protein, 274 aa, Homo sapiens
SGLNDIFEAQKIEWHEVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 171 - protein, 451 aa, Artificial Sequence
  Description of Artificial Sequence Synthetic polypeptide
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 214 aa, Artificial Sequence; identical to SEQ ID NO: 168
  Description of Artificial Sequence Synthetic polypeptide

SEQ ID NO: 173 - protein, 451 aa, Artificial Sequence
  Description of Artificial Sequence Synthetic polypeptide
EVQLQQSGAVLARPGASVKMSCKGSGYTFTNYWMHWVKQRPGQGLEWIGATYRGHSDTYYNQKFKGKAKLTAVTSTSTAYMELSSLTNEDSAVYYCTRGAIYNGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 215 aa, Artificial Sequence
  Description of Artificial Sequence Synthetic polypeptide
DIQLTQTTSSLSASLGDRVTISCSASQDISNYLNWYQQKPDGTVELVIYYTSNLHSGVPSRFSGSGSGTDYSLTIGYLEPEDVATYYCQQYRKLPWTFGGGSKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 451 aa, Artificial Sequence
  Description of Artificial Sequence Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 214 aa, Artificial Sequence; identical to SEQ ID NO: 164
  Description of Artificial Sequence Synthetic polypeptide

SEQ ID NO: 177 - protein, 13 aa, Artificial Sequence
  Description of Artificial Sequence Synthetic peptide
ELAGSSYEGYFES

SEQ ID NO: 178 - protein, 13 aa, Artificial Sequence
  Description of Artificial Sequence Synthetic peptide
ELSASSYEGYFES

SEQ ID NO: 179 - protein, 13 aa, Artificial Sequence
  Description of Artificial Sequence Synthetic peptide
ELSGASYEGYFES

SEQ ID NO: 180 - protein, 13 aa, Artificial Sequence
  Description of Artificial Sequence Synthetic peptide
ELSGSAYEGYFES

SEQ ID NO: 181 - protein, 13 aa, Artificial Sequence
  Description of Artificial Sequence Synthetic peptide
ELSGSSAEGYFES

SEQ ID NO: 182 - protein, 13 aa, Artificial Sequence
  Description of Artificial Sequence Synthetic peptide
ELSGSSYAGYFES

SEQ ID NO: 183 - protein, 13 aa, Artificial Sequence
  Description of Artificial Sequence Synthetic peptide
ELSGSSYEAYFES

SEQ ID NO: 184 - protein, 13 aa, Artificial Sequence
  Description of Artificial Sequence Synthetic peptide
ELSGSSYEGAFES

SEQ ID NO: 185 - protein, 13 aa, Artificial Sequence
  Description of Artificial Sequence Synthetic peptide
ELSGSSYEGYAES

SEQ ID NO: 186 - protein, 13 aa, Artificial Sequence
  Description of Artificial Sequence Synthetic peptide
ELSGSSYEGYFAS

SEQ ID NO: 187 - protein, 13 aa, Artificial Sequence
  Description of Artificial Sequence Synthetic peptide
ELSGSSYEGYFEA

SEQ ID NO: 188 - protein, 10 aa, Artificial Sequence
  Description of Artificial Sequence Synthetic peptide
AGYYSGGSYA

SEQ ID NO: 189 - protein, 10 aa, Artificial Sequence
  Description of Artificial Sequence Synthetic peptide
QAYYSGGSYA

SEQ ID NO: 190 - protein, 10 aa, Artificial Sequence
  Description of Artificial Sequence Synthetic peptide
QGAYSGGSYA

SEQ ID NO: 191 - protein, 10 aa, Artificial Sequence
  Description of Artificial Sequence Synthetic peptide
QGYASGGSYA

SEQ ID NO: 192 - protein, 10 aa, Artificial Sequence
  Description of Artificial Sequence Synthetic peptide
QGYYAGGSYA

SEQ ID NO: 193 - protein, 10 aa, Artificial Sequence
  Description of Artificial Sequence Synthetic peptide
QGYYSAGSYA

SEQ ID NO: 194 - protein, 10 aa, Artificial Sequence
  Description of Artificial Sequence Synthetic peptide
QGYYSGASYA

SEQ ID NO: 195 - protein, 10 aa, Artificial Sequence
  Description of Artificial Sequence Synthetic peptide
QGYYSGGAYA

SEQ ID NO: 196 - protein, 10 aa, Artificial Sequence
  Description of Artificial Sequence Synthetic peptide
QGYYSGGSAA

SEQ ID NO: 197 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARALSGSSYEGYFESWGQGTLVTVSS

SEQ ID NO: 198 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCAREASGSSYEGYFESWGQGTLVTVSS

SEQ ID NO: 199 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELAGSSYEGYFESWGQGTLVTVSS

SEQ ID NO: 200 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSASSYEGYFESWGQGTLVTVSS

SEQ ID NO: 201 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGASYEGYFESWGQGTLVTVSS

SEQ ID NO: 202 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSAYEGYFESWGQGTLVTVSS

SEQ ID NO: 203 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSAEGYFESWGQGTLVTVSS

SEQ ID NO: 204 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYAGYFESWGQGTLVTVSS

SEQ ID NO: 205 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEAYFESWGQGTLVTVSS

SEQ ID NO: 206 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGAFESWGQGTLVTVSS

SEQ ID NO: 207 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYAESWGQGTLVTVSS

SEQ ID NO: 208 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFASWGQGTLVTVSS

SEQ ID NO: 209 - protein, 123 aa, Artificial Sequence
  Description of Artificial Sequence Synthetic polypeptide
EVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFEAWGQGTLVTVSS

SEQ ID NO: 210 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCAGYYSGGSYAFGGGTKVEIK

SEQ ID NO: 211 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQAYYSGGSYAFGGGTKVEIK

SEQ ID NO: 212 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGAYSGGSYAFGGGTKVEIK

SEQ ID NO: 213 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYASGGSYAFGGGTKVEIK

SEQ ID NO: 214 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYAGGSYAFGGGTKVEIK

SEQ ID NO: 215 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSAGSYAFGGGTKVEIK

SEQ ID NO: 216 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGASYAFGGGTKVEIK

SEQ ID NO: 217 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGAYAFGGGTKVEIK

SEQ ID NO: 218 - protein, 110 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSGGSAAFGGGTKVEIK

SEQ ID NO: 219 - protein, 13 aa, Artificial Sequence
  Description of Artificial Sequence Synthetic peptide
ALSGSSYEGYFES

SEQ ID NO: 220 - protein, 13 aa, Artificial Sequence
  Description of Artificial Sequence Synthetic peptide
EASGSSYEGYFES

SEQ ID NO: 221 - protein, 18 aa, Artificial Sequence
  Description of Artificial Sequence Synthetic peptide
CIYSPSGDIKYYADWAKG

SEQ ID NO: 222 - protein, 484 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCAGYYSGGSYAFGGGTKVEIKGGGGSGGGGSGGGGSEVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 223 - protein, 484 aa, Artificial Sequence
  Description of Artificial Sequence Synthetic polypeptide
DIQLTQSPSTLSASVGDRVTITCRSSQSVYGHNWLAWYQQKPGKAPKLLMYRASNLASGVPSRFSGSGSGTEFTLTISSVQPDDFATYYCQGYYSAGSYAFGGGTKVEIKGGGGSGGGGSGGGGSEVQLLESGGGLVQPGGSLRLSCAASGFSFNNYWIIWIRQAPGKGLEWVAAIYSPSGDIKYYADWAKGRFTVSKDNSKNTVYLQMNSLRAEDTAVYYCARELSGSSYEGYFESWGQGTLVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 224 - protein, 452 aa, Artificial Sequence
  Description of Artificial Sequence Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK